(19) United States
(12) Patent Application Publication
(10) Pub. No.: US 2026/0325508 A1
(43) Pub. Date: Oct. 22, 2026

(54) COMBINATION OF PAC-1 AND CAR T-CELL TREATMENTS

(71) Applicant: THE BOARD OF TRUSTEES OF THE UNIVERSITY OF ILLINOIS, Urbana, IL (US)

(72) Inventors: Paul J. HERGENROTHER, Champaign, IL (US); Diana Rose E. RANOA, Champaign, IL (US); David M. KRANZ, Champaign, IL (US)

(21) Appl. No.: 19/484,565

(22) PCT Filed: May 22, 2024
(86) PCT No.: PCT/US2024/030595

§ 371 Date: Nov. 14, 2025

Related U.S. Application Data
(60) Provisional application No. 63/468,104, filed on May 22, 2023.

(51) Int. Cl. A61K 31/495; A61K 40/11; A61K 40/31; A61K 40/42; A61P 35/00

(52) U.S. Cl. CPC A61K 31/495; A61K 40/11; A61K 40/31; A61K 40/4259; A61P 35/00

(57) ABSTRACT

The disclosure provides for composition comprising the compound PAC-1 and a population of T-cells comprising a chimeric antigen receptor (CAR) fusion protein displayed on a cell surface membrane of the T-cells, wherein the CAR fusion protein selectively binds to Tn-antigen of a protein disposed on a surface of a tumor cell. Also provided are methods of using PAC-1 and T-cells to treat various diseases such as, but not limited to, cancer.

### RELATED APPLICATIONS

This application claims priority under 35 U.S.C. § 119(e) U.S. Provisional Patent Application No. 63/468,104, filed May 22, 2023, which is incorporated herein by reference.

### GOVERNMENT SUPPORT

This invention was made with government support under Grant No. R01 CA120439 awarded by the National Institutes of Health. The government has certain rights in the invention.

### BACKGROUND OF THE INVENTION

Adoptive therapies with T cells that express chimeric antigen receptors (CARs) against hematopoietic cancers have shown tremendous promise. The success of these CAR-T cell therapies has stemmed in part from the targeting of the normal B cell-specific differentiation antigen, CD19, which allows elimination of malignant B cells. In contrast, solid tumors have been difficult to treat with CAR-T cells because of a lack of suitable targets and unique immunosuppressive environments. Hematopoietic cancers offer other B cell-specific differentiation antigens, but targeting self-antigens (e.g. HER2, folate receptor-α, mesothelin, MUC16) expressed on non-hematopoietic cancers has been limited by toxicity to normal tissues that express the same targets.

Ovarian cancer is a particularly devastating disease, with a poor prognosis due to the lack of early diagnostics. Treatment involves surgery and chemotherapy (typically platinum-based drugs) and, more recently, inhibitors of angiogenesis and poly(ADP-ribose) polymerase-1 (PARP-1). Despite these approaches, five-year survival statistics have seen minimal improvement. While there are CARs in development against various ovarian cancer antigens, to date there have not been clinically effective results. Although preclinical effective CARs have been identified, these typically involved immunodeficient mouse models that did not express the human target.

### SUMMARY

Recently, we focused on CARs 237 and 5E5 that recognize specific Tn-glycopeptide-antigens (GalNAcα1-O-Ser/Thr-protein). The most studied Tn-antigens are the heavily O-glycosylated, repeat proteins of the Mucin (MUC) family. MUC1 has recently been shown to induce affinity-matured antibodies, but these and other antibodies bound only to non-glycosylated MUC1. In contrast, antibodies 237 and 5E5 are cancer-specific and require GalNAc in combination with peptide epitopes. Targeting Tn-peptide antigens has the potential to avoid antigen-loss variants as observed when targeting protein epitopes such as CD19, as the glycosylation defect is often critical for tumor cell function and Tn-dependent CARs that recognize multiple different protein backbones can be generated.

The 237 and 5E5 antibodies were generated against the Tn-antigen linked to their cognate proteins, mouse podoplanin (also known as OTS8) and human MUC1, respectively. The cancer-specific expression of Tn-antigens can result from various dysregulated steps of the pathway, including the glycosyltransferase enzymes or the chaperone Cosmc. While Cosmc mutations are found in approximately 1 to 5% of cancers, other mechanisms of O-linked glycosylation dysregulation appear to contribute to estimates that 85% of ovarian cancers express the epitope recognized by the Tn-MUC1 antibody 5E5.

Many previous studies have used the mouse tumor line ID8 as a model for human ovarian cancer. Various treatment approaches, including surgery, cisplatin, checkpoint inhibitors, oncolytic viruses, and PARP-1 inhibitors have been tested in the ID8 model, but tumor control in these studies has required that treatments be given early, before the presence of established, disseminated tumors. Our approaches here, with mouse CAR-T cells against ID8, focus on treatment when there are well-established solid tumors in a syngeneic system that is intended to resemble the scenario encountered in human disease.

This disclosure shows that a single 237 CAR-T cell treatment of ID8Cosmc-KO-bearing mice, i.v. on day 40 after tumor transplantation, resulted in long-term delay of tumor growth. Interestingly, i.p. administration of the CAR-T cells resulted in improved efficacy, with delayed progression when given as late as 60 days after tumor inoculation. At this point, mice exhibited extensive dissemination of solid tumors in the peritoneal cavity. A comparison of four Tn-dependent CARs showed that maximal efficacy required a minimal affinity for the Tn-OTS8 antigen. Among the four CARs, there was a distinct profile of inflammatory cytokine release, highlighted by the observation that the least effective CAR (5E5) exhibited tonic signaling leading to a higher basal level of cytokine expression and antigen-induced secretion of IL-10. We also describe long-term persistence of the CAR-T cells, and their ability to delay growth of an ID8Cosmc-KO tumor re-challenge. These findings support the potential of treating ovarian cancer with affinity-optimized CAR-T cells, using intraperitoneal routes of administration for optimal effectiveness.

Accordingly, the disclosure provides for a composition comprising: the compound PAC-1:

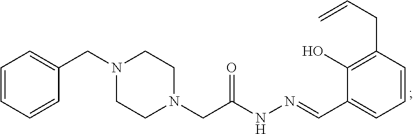 [SMILES: C=CCc1cccc(/C=N/NC(=O)CN2CCN(Cc3ccccc3)CC2)c1O]

and
a population of T-cells comprising a chimeric antigen receptor (CAR) fusion protein displayed on a cell surface membrane of the T-cells, wherein the CAR fusion protein selectively binds to an N-acetylgalactosamine-O-Serine or N-acetylgalactosamine-O-Threonine antigen moiety (Tn-antigen) of a protein disposed on a surface of a tumor cell. In some embodiments, the CAR fusion protein comprises a single-chain variable fragment (scFv) antibody that selectively binds to the Tn-antigen.

In some embodiments, the CAR fusion protein comprises a formula A-B-C-D, wherein A is the scFv, B is the hinge region, C is the transmembrane domain, and D is the cytoplasmic domain. In some embodiments, the CAR fusion protein comprises an amino acid sequence of SEQ ID NO: 5, SEQ ID NO: 6, SEQ ID NO: 7, or SEQ ID NO: 8; or the fusion protein comprises an amino acid sequence of SEQ ID NO: 1, SEQ ID NO: 2, SEQ ID NO: 3, or SEQ ID NO: 4.

In some embodiments, the population of T-cells in a composition comprises about 5×10⁵ T-cells to about 5×10⁸ T-cells, or about 5×10⁶ T-cells. In some embodiments, composition comprises a PAC-1 concentration of about 0.1 μM to about 50 μM.

The disclosure also provides for methods of treating cancer comprising administering to a subject in need thereof, concurrently or sequentially, a therapeutically effective amount of PAC-1 and a therapeutically effective amount of a population of chimeric antigen receptor (CAR) T-cells wherein the effect of the population of CAR T-cells is enhanced by the administration of the PAC-1, and wherein the population of CAR T-cells comprise a CAR fusion protein that selectively binds to an N-acetylgalactosamine-O-Serine or N-acetylgalactosamine-O-Threonine antigen moiety (Tn-antigen) of a protein disposed on a surface of a tumor cell.

In some embodiments, the PAC-1 is administered in a dose comprising about 10 mg/kg to about 125 mg/kg; and/or the population of CAR T-cells are administered in a dose comprising about 5×10⁵ CAR T-cells to about 5×10⁸ CAR T-cells.

In some embodiments, the dual PAC-1 and CAR T-cell therapy may be used to treat various cancers such as embryonal tumor, esophagogastric cancer, central nervous system cancer, bladder cancer, breast cancer, glioblastoma, head and neck cancer, hepatobiliary cancer, lung cancer, melanoma, meningioma, multiple myeloma, ocular melanoma, ovarian cancer, ovarian epithelial cancer, osteosarcoma, pancreatic cancer, prostate cancer, renal cancer, salivary gland cancer, sarcoma, seminoma, or soft tissue sarcoma. In some embodiments, the cancer is a solid tumor. In other embodiments, the cancer is ovarian cancer.

In some embodiments, the compound PAC-1 and the population of CAR T-cells are concurrently administered to the subject; or the compound PAC-1 and the population of CAR T-cells are sequentially administered to the subject, wherein the compound PAC-1 is administered to the subject before the population of T-cells or the compound PAC-1 is administered to the subject after the population of CAR T-cells.

In some embodiments, the compound PAC-1 is administered to the subject once daily (q.d.), twice a day (b.i.d.), three times a day (t.i.d.), or four times a day (q.i.d.), wherein the total administered dose per day of PAC-1 is about 1 mg/kg to about 150 mg/kg; or each administered dose of PAC-1 is about 70 mg, about 175 mg, about 250 mg, about 375 mg, about 450 mg, about 500 mg, about 625 mg, about 750 mg, or about 800 mg; or each administered dose of PAC-1 is about 50 mg/m² to about 250 mg/m². In some embodiments, the compound PAC-1 is administered over the course of three administration time periods, wherein each of the administration time periods is 5 consecutive days with at least two days between each of the administration time periods, wherein the PAC-1 is administered once daily for each of the 5 consecutive days; and the population of T-cells are administered during one of a second or third administration time period.

In some embodiments, a method of treating ovarian cancer comprising administering to a human in need thereof, concurrently or sequentially, a therapeutically effective amount of: PAC-1; and a population of T-cells comprising a chimeric antigen receptor (CAR) fusion protein displayed on a cell surface membrane of a T-cell, wherein the CAR fusion protein comprises one or more of a 237 single chain variable fragment (scFv) (e.g., SEQ ID NO: 1), a WE single chain variable fragment (scFv) (e.g., SEQ ID NO: 2), a TNGK single chain variable fragment (scFv) (e.g., SEQ ID NO: 3), and a LGQ single chain variable fragment (scFv) (e.g., SEQ ID NO: 4), wherein each of the 237 scFv, the WE scFv, the LGQ scFv, and the TNGK scFv selectively bind to protein comprising an N-acetylgalactosamine-O-Serine or N-acetylgalactosamine-O-Threonine antigen disposed on a surface of an ovarian cancer cell, wherein the combination of PAC-1 and the population of T-cells treat the ovarian cancer.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the disclosure. In some instances, embodiments of the disclosure can be best understood by referring to the accompanying drawings in combination with the detailed description presented herein. The description and accompanying drawings may highlight a certain specific example, or a certain aspect of the disclosure. However, one skilled in the art will understand that portions of the example or aspect may be used in combination with other examples or aspects of the disclosure.

FIG. 1A-C. ID8Cosmc-KO as a model for ovarian cancer in C57BL/6J mice. Tn antigen expression on ID8Cosmc-KO was measured by flow cytometry using either (A) staining with monoclonal mouse IgM anti-Tn antibody 5F4 followed by goat anti-mouse IgM conjugated to AlexaFluor 647 or (B) biotinylated anti-Tn-OTS8 WE scFv tetramer containing streptavidin-PE. (C) ID8 WT and ID8Cosmc-KO cell doubling times (19.2 h and 21.8 h, respectively) in vitro were measured over time by manual cell counting. Growth curves from two different experiments (total N=6) were combined and plotted using GraphPad Prism v9.4.1 and cell doubling times were calculated using the exponential growth equation.

FIG. 2A-E. CAR-T cell treatment of mice with established ID8Cosmc-KO tumors. (A) Schematic of 237 single-chain Fv linked by a CD8 hinge-CD28 transmembrane domain to the CD28 and CD3ζ cytoplasmic domains, cloned into the pMP71 retroviral vector. (B) Primary T cells isolated from splenocytes of naïve donor C57BL/6J mice were transduced with 237 CAR for 72 hours. Transduction efficiency was measured by flow cytometry using tetramers of biotinylated/Tn-glycosylated OTS8 peptide made with streptavidin-PE. Mock-transduced T cells were included as negative control. (C, D) Survival curves of ID8Cosmc-KO-bearing C57BL/6J mice either untreated, treated with mock-transduced T cells, or 237 CAR-T cells administered by retro-orbital intravenous injection of 5×10⁶ T cells at day 40 (C) or day 56 (D) post-tumor inoculation. Note that five of the mice in the 237 CAR group (C) were used at day 160 for a re-challenge experiment (FIG. 7B), such that their survival times may have been even longer without this re-challenge. (E) Comparison of intravenous (dashed line) and intraperitoneal (solid line) delivery of 237 CAR-T cells at day 50 post-tumor inoculation. All survival curves were plotted with GraphPad Prism V.9.4.1 using cumulative data across multiple experiments (two combined experiments for C and D, four combined experiments for E), with the indicated total n for each treatment arm. Median survival times are indicated; p values were calculated using the log-rank (Mantel-Cox) test. CAR, chimeric antigen receptor; i.p., intraperitoneal; i.v., intravenous; KO, knock-out.

FIG. 3A-C. Efficacy of engineered CAR T constructs with different affinities for the mouse Tn-OTS8 antigen in the ID8Cosmc-KO ovarian tumor model. (A) Survival curves of ID8Cosmc-KO-bearing C57BL/6J mice treated with 237 CAR T, TNGK CAR T, or 5E5 CAR T by i.v. injection of 5×10⁶ transduced T cells at day 40 post-tumor inoculation. (B, C) Survival curves of ID8Cosmc-KO-bearing C57BL/6J mice treated with different CAR-T cells by i.p. injection of 5×10⁶ transduced T cells at day 50 (B) or day 60 (C) post tumor-inoculation. All survival curves were plotted with GraphPad Prism v9.4.1 using cumulative data across multiple experiments (two combined experiments for A and C, four for B) with the indicated total n for each treatment arm. Median survival times are indicated; P-values were calculated using the Log-rank (Mantel-Cox) test.

FIG. 4A-H. In vitro cytokine and killing analyses of different CAR T cells. Mock, 237-CAR, WE-CAR, TNGK-CAR, or 5E5-CAR transduced primary T cells from C57BL/6J donor mice were co-cultured with ID8Cosmc-KO cells in a 96-well plate for 24 hours at various tumor:effector ratios. The number of effector T cells were kept constant at 12,000 cells/well across all conditions tested. After 24 hours, 25 μL culture supernatants were analyzed for multiple cytokines using a Luminex plate reader to measure IFN-γ (A), IL-2 (B), IL-6 (C), TNFα (D), IL-4 (E), and IL-10 (F). Plots shown are combined cytokine array data points from five experiments. Bar graphs were plotted using GraphPad Prism v9.4.1. Error bars are SEM. To simplify the presentation, P-values are shown only for 237 versus 5E5 CAR. A complete set of tumor:effector ratio titration plots for each of the five cytokine array experiments are shown in FIG. 12. (G, H) The remaining ID8Cosmc-KO (G) or ID8 WT (H) adherent tumor cells on plates were fixed in ice-cold 10% trichloroacetic acid for the SRB viability assay. The percent growth inhibition was calculated using the formula outlined in Methods section (Example 2). SRB experiments were performed twice, each with a technical replicate of n=4. Bar graphs were plotted using GraphPad Prism v9.4.1. Error bars are SEM. P-values for 237, WE, TNGK were calculated against either mock or 5E5 at <0.0001 (or denoted by ***); P-values for 5E5 against either mock T or TNGK are at <0.001 (**).

FIG. 5A-C. Peritoneal organs were harvested from ID8Cosmc-KO inoculated mice, three days after i.p. treatment with either 237 CAR-T or 5E5 CAR-T at day 54 post-tumor inoculation. Untreated mice were used as control. ImageJ-quantified CD3⁺ T cells (A), F4/80⁺ macrophages (B), and cleaved Caspase-3⁺ apoptotic cells (C) from tissue sections of untreated, 237 CAR-T, 5E5 CAR-T treated mice. A total of 5-20 images at 20× magnification corresponding to tumors spotted lining the fatty tissues near stomach, small and large intestines of 2 mice per treatment group were analyzed and quantified using ImageJ. Dot plots and P-values were generated using GraphPad Prism v9.4.1. Error bars are SEM. Each dot represents one field of view at 20× magnification.

FIG. 6A-E. Evidence for in vivo persistence of 237 CAR T cells. (A) 237 CAR T-treated ID8Cosmc-KO-bearing mice were re-challenged with 10⁷ ID8Cosmc-KO cells at 120 days post-T cell transfer. Age-matched naïve mice were inoculated with 10⁷ ID8Cosmc-KO cells and used as control. Blood samples were collected from some mice immediately before i.p. injection of re-challenged tumors and 7 days after re-challenge; processed and stained with anti-CD3 antibody and Tn-glycosylated OTS8 peptide tetramer, and analyzed by flow cytometry. The percentage of CD3⁺ 237 CAR T cells were plotted using GraphPad Prism v9.4.1. Each dot represents one mouse. Error bars are SEM. (B) Survival curve of 237 CAR T treated mice re-challenged with ID8Cosmc-KO at either 120 (n=5) or 160 days (n=5) after initial tumor injection, compared to age-matched C57BL/6 naïve mice that were inoculated with the same number of tumor cells. (C) Spleens were removed from 237 CAR T-treated ID8Cosmc-KO bearing mice up to 5 months after CAR treatment (152 days for the data shown). Spleen cells were co-cultured with either ID8 WT or ID8Cosmc-KO tumor cells in a 96-well plate at a tumor:effector ratio of 0.3. ELISPOT assays were performed to measure IFN-gamma spots 24 hours after co-culture experiment. Splenocytes from age-matched mice were used as a control, while CD3/CD28 Dynabeads were added to T cells as a positive control for T cell activation. Plates were scanned with automated ImmunoSpot analyzer. Bar graph of quantified IFN-γ spots are plotted on the right panel. Results representative of five independent experiments. (D) Non-tumor bearing mice were injected intraperitoneally with 237 CAR T (See FIG. 9B) or untreated. At day 60, mice were challenged with 10⁷ ID8Cosmc-KO cells and the survival curve was compared to that of mice inoculated with tumor that did not receive prior 237 CAR T injections (untreated). (E) Survival curve of ID8 WT-bearing C57BL/6J mice either untreated, treated with mock-transduced T cells, or treated with 237 CAR T, administered by i.p. injection of 5×10⁶ T cells at day 60. Data for D and E are from two combined experiments. All survival curves were plotted using GraphPad Prism v9.4.1 with the indicated total n for each treatment arm. Median survival times are indicated; P-values were calculated using the Log-rank (Mantel-Cox) test.

FIG. 7A-B. Weights of C57BL/6J mice administered 237 CAR T in tumor-bearing (A) and non-tumor bearing (B) C57BL/6J mice. (A) ID8Cosmc-KO-innoculated mice were administered 5×10⁶ 237 or WE CAR-T cells (or mock-transduced T cells) at day 50 via i.p. injection. (B) Non-tumor bearing mice were administered 5×10⁶ 237 CAR-T cells (or mock-transduced T cells) by i.p. injection. Body weights were measured over a period of 60 days post-T cell transfer. Line graphs were plotted using GraphPad Prism v9.4.1. Each line represents 1 mouse.

FIG. 8A-D. Binding of biotinylated Tn-peptides to the four CAR-T cells. (A) Mock, 237, WE, TNGK, and 5E5 CAR-T cells were stained with various concentrations of biotinylated Tn-OTS8 peptide (A) or Tn-MUC1 peptide (B), followed by streptavidin-PE. Mean fluorescent intensity (MFI) plotted against peptide concentration illustrates the affinity of CAR-T cells for their respective peptide Tn-antigens. MFI values were taken from Sharma et al., PNAS 2020 PMID: 32541028. Plots shown are representative of two independent experiments. (C) Binding of (C) 237, WE, and (D) TNGK, and 5E5 CAR-Ts to either 10 nM biotinylated Tn-OTS8 tetramer prepared with streptavidin-PE (top row) or 100 nM biotinylated Tn-MUC1 followed by streptavidin-Alexafluor647 (bottom row) for both (C) and (D). Flow cytometry plots were overlaid against mock-transduced T cells. The percentage of positive peaks and their MFI are indicated for each plot.

FIG. 9A-B. Comparison of 237, WE, TNGK and 5E5 CAR T cells in killing assays in vitro (growth inhibition). ID8Cosmc-KO (A) and ID8 WT (B) were examined with mock, 237-CAR, WE-CAR, TNGK-CAR, or 5E5-CAR transduced primary T cells from C57BL/6J mice, co-cultured with either ID8Cosmc-KO or ID8 WT cells in a 96-well plate for 24 hours at various tumor:effector ratios. The number of tumor cells were kept constant at 6,000 cells/well across all conditions tested. After 24 hours, culture supernatants were removed, and the adherent cells were fixed in ice-cold 10% trichloroacetic acid for the SRB assay. The % growth inhibition was calculated using the formula outlined in the Methods section. This experiment was performed twice, each with a technical replicate of n=4. Representative line graphs were plotted using GraphPad Prism v9.4.1. Error bars are SEM. Error bars are SEM. P-values for 237, WE, TNGK were calculated against either mock or 5E5 at <0.0001 (or denoted by ***); P-values for 5E5 against either mock T or TNGK are at <0.001 (**).

FIG. 10A-F. Antigen-specific proliferation of CFSE-labeled CAR-T cells. Mock, 237, WE, TNGK, 5E5 CAR-Ts were stimulated with various concentrations of OTS8, Tn-OTS8, MUC1, Tn-MUC1 peptides coated on streptavidin immobilized plates. After 72 hours, cells were collected and analyzed by forward versus side scatter gating on the size of lymphocytes. (A) Representative images of forward versus side scatter gating of 237 CAR-T cells stimulated with either OTS8 or Tn-OTS8 peptide (A). (B) The percentage of viable T cells in the forward/side scatter gate were plotted against the various concentration of (Tn)-peptides used for stimulation. (C) CFSE fluorescence of cells collected for scatter were also analyzed to determine the level of proliferation of viable cells. Flow histograms with indicated MFI for each sample are shown. (D)-(F) Bar graphs of MFI from CFSE histograms in (C) are shown. Duplicate samples from one experiment were plotted. Lower MFI reflects greater extent of proliferation. Representative of 3 independent experiments, each performed in duplicate. For (B), closed circle=mock T, open circle=237 CAR T, open square=WE CAR T, open triangle=TNGK CAR T; open diamond=5E5 CAR T. For (D)-(F), closed circle=OTS8, open circle=Tn-OTS8, closed square=MUC-1, open square=Tn-MUC-1.

FIG. 11A-C. Immune cell infiltration in tumors of CAR-treated mice. (Immunophenotyping of blood, spleen, tumor nodules in pancreas, and ascites fluid from ID8Cosmc-KO-bearing C57BL/6J mice, three days post-treatment with either 237 or 5E5 CAR-T (5×10⁶ cells) i.p. CAR T cells were administered at day 54 post-tumor challenge. Age-matched non-tumor bearing mice were included as controls, in addition to an untreated ID8Cosmc-KO inoculated cohort. Dissociated cells were stained with a cocktail containing anti-CD45, anti-CD3, anti-CD8, and anti-CD4 antibodies and a viability dye. The presence of 237 CAR T in circulation and in tumors was measured using tetramers of biotinylated Tn-OTS8 peptide included in the antibody cocktail used to stain dissociated cells from tissue samples. Percent cell populations of CD3+ (A), CD8+ (B), and CD4+ T cells (C) were calculated from flow cytometry data obtained using BD LSRII and analyzed using FlowJo software, with n=3 for each tumor-bearing group or n=2 for non-tumor bearing group treated with CAR-Ts. Dot plots were generated using GraphPad Prism v9.4.1. Error bars are SEM.

FIG. 12A-B. Evidence for in vivo persistence of 237 CAR T cells. (A) Blood samples were collected at 55 days post-re-challenge of surviving mice, and stained for the percentage of persistent 237 CAR T cells (Tn-OTS8⁺) among the population of CD3⁺ (left panel), CD4⁺ (middle panel), or CD8⁺ cells (right panel). (B) IFN-γ ELISPOT of splenic T cells from untreated or CAR treated mice co-cultured with tumor cells. Spleens were removed from 237 CAR T-treated ID8Cosmc-KO bearing mice up to 5 months after CAR treatment (226 days for the data shown). Spleen cells were co-cultured with various C57BL/6 syngeneic cell lines including ID8 WT, ID8 Cosmc-KO, AG104A (Tn-OTS8+), GL261, B16F10, and LLC1 tumor cells in a 96-well plate at a tumor:effector ratio of 0.3. ELISPOT assays were performed to measure IFN-γ spots 24 hours after co-culture experiment. Splenocytes from 5E5 CAR-T treated mouse or 237 CAR-T treated ID8 WT-challenged mouse that reached endpoint were used as controls, while CD3/CD28 Dynabeads were added to T cells as a positive control for T cell activation. Plates were scanned with automated ImmunoSpot analyzer. Results representative of five total experiments (with ID8Cosmc-KO and ID8-WT). Data were plotted using GraphPad Prism v9.4.1, each dot represents one mouse. Error bars are SEM.

FIG. 13A-C. Tn-antigen expression levels on tumors from CAR-treated, long-term, relapsed mice. (A) Ascites collected from ID8Cosmc-KO bearing mice that reached endpoint were stained with tetramers of biotinylated WE-scFv to examine the fraction of ascites cells that represent the antigen-positive tumor. Data were plotted according to treatment groups using GraphPad Prism v9.4.1. Each dot represents one mouse. Error bars are SEM. (B)-(C) Cells from ascites collected from various mice (untreated or CAR-treated) that reached endpoint criterion were grown in culture for 21 days to generate tumor cell lines. Cultured lines were stained with biotinylated WE-scFv followed by streptavidin-Alexafluor647. Histograms were overlaid against reference ID8 WT and ID8Cosmc-KO cell lines. Dashed line=ID8 WT, dotted line=ID8 Cosmc-KO, solid line=tumor cells from ascites, cultured for 21 days.

FIG. 14A-B. PAC-1 enhanced efficacy of 237 CAR-T in late-stage ovarian cancer model. Survival curves of mice with established ID8Cosmc-KO tumors treated with a combination of PAC-1 and 237 CAR-T in comparison with their single-agent controls. (A) Mice with detectable tumors at day 40 post-tumor inoculation were administered with either PAC-1 (100 mg/kg) or vehicle intraperitoneally for three weeks at a “five days ON, 2 days OFF” treatment schedule. At day 60 post-tumor inoculation, mice were given either 237 CAR-T or mock T cells (5 million cells, >40% transduction efficiency) via i.p. injection. Cyclophosphamide (100 mg/kg, i.p.) were administered 24 hours prior to adoptive T cell transfer to deplete pre-existing lymphocytes. (B) Data were plotted with GraphPad Prism v10 using cumulative data across five independent experiments, with the indicated total n for each treatment arm. Median survival times are indicated; P-values were calculated using the Log-rank (Mantel-Cox) test.

FIG. 15A-B. PAC-1 at day 50 (or day 60) and 237 CAR-T at day 60 combination treatment. Survival curves of mice with established ID8 Cosmc-KO tumors treated with a combination of PAC-1 and 237 CAR-T in comparison with their single-agent controls. (A) Mice with tumors at either day 50 or day 60 post-tumor inoculation were administered with either PAC-1 (100 mg/kg) or vehicle intraperitoneally for three weeks at a “five days ON, 2 days OFF” treatment schedule. At day 60 post-tumor inoculation, mice were given either 237 CAR-T or mock T cells (5 million cells, >40% transduction efficiency) via i.p. injection. Cyclophosphamide (100 mg/kg, i.p.) were administered 24 hours prior to adoptive T cell transfer to deplete pre-existing lymphocytes. Data were plotted with GraphPad Prism v10 using cumulative data across multiple experiments, with the indicated total n for each treatment arm. Median survival times are indicated; P-values were calculated using the Log-rank (Mantel-Cox) test. Mice with tumors at either day 50 (B) or day 60 (C) post-tumor inoculation.

FIG. 16A-B. 237 CAR-T at day 60 and PAC-1 at day 77 combination treatment. Survival curves of mice with established ID8 Cosmc-KO tumors treated with a combination of 237 CAR-T and PAC-1. (A) Mice with tumors at day 60 post-tumor inoculation were given either 237 CAR-T or mock T cells (5 million cells, >40% transduction efficiency) via i.p. injection. Cyclophosphamide (100 mg/kg, i.p.) were administered 24 hours prior to adoptive T cell transfer to deplete pre-existing lymphocytes. At day 77 or 78 post-tumor inoculation, mice were administered with PAC-1 (100 mg/kg) intraperitoneally for three weeks at a “five days ON, 2 days OFF” treatment schedule. (B) Data were plotted with GraphPad Prism v10 using cumulative data from 2 independent experiments, with the indicated total n for each treatment arm. Median survival times are indicated; P-values were calculated using the Log-rank (Mantel-Cox) test.

FIG. 17A-C. Survival curves of mice with established ID8Cosmc-KO tumors treated with a combination of: (A) Doxorubicin and 237 CAR-T; (B) Anti-PD1 and 237 CAR-T; and (C) PAC-1, anti-PD1, and 237 CAR-T. In all combinations tested, no significant difference was observed when compared against 237 CAR-T administered as a single agent. A) Mice with tumors at day 40 post-tumor inoculation were administered with either Doxorubicin (7.5 mg/kg) or vehicle intraperitoneally once a week for three weeks (at days 40, 47, and 54). B) Mice with tumors at day 41 post-tumor inoculation were administered with either anti-PD1 monoclonal antibody clone RMP1-14 (200 μg) or the corresponding IgG2a isotype control clone 2A3 (200 μg) intraperitoneally once every 4 days for a total of four doses (given at days 41, 45, 49, and 53). C) Mice with tumors at day 40 post-tumor inoculation were administered with either PAC-1 (100 mg/kg)+anti-PD1 monoclonal antibody clone RMP1-14 (200 μg) or vehicle+anti-PD1 monoclonal antibody (200 μg) or vehicle+IgG2a isotype control clone 2A3 (200 μg) intraperitoneally for three weeks at a “five days ON, 2 days OFF” treatment schedule for PAC-1 and once every 4 days for a total of four doses (given at days 41, 45, 49, and 53) for the monoclonal antibodies. In all combinations tested, mice were given either 237 CAR-T or mock T cells (5 million cells, >40% transduction efficiency) at day 60 post-tumor inoculation via i.p. injection. Cyclophosphamide (100 mg/kg, i.p.) were administered 24 hours prior to adoptive T cell transfer to deplete pre-existing lymphocytes. Data were plotted with GraphPad Prism v10 using cumulative data across multiple experiments, with the indicated total n for each treatment arm. Median survival times are indicated; P-values were calculated using the Log-rank (Mantel-Cox) test.

FIG. 18A-B. Persistence of CAR-Ts: T cells isolated from CAR-T treated mice recognized Tn-expressing cell lines in in vitro co-culture assays. (A) T cells were isolated from the spleens of 237 CAR-T treated and PAC-1+237 CAR-T treated ID8Cosmc-KO bearing mice sacrificed at the indicated days post-tumor inoculation. Purified T cells were then co-cultured with various murine (ID8 WT, ID8 Cosmc-KO) and human (SKOV3, SKOV3 COSMC-KO MUC1-KO, and LOX-IMVI) cell lines in a 96-well plate at a tumor:effector ratio of 0.3. ELISPOT assays were performed to measure IFN-gamma spots 24 hours after co-culture experiment. T cells isolated from the spleen of a naïve/non-tumor bearing mouse were used as a negative control, while CD3/CD28 Dynabeads were added to designated wells as a positive control for T cell activation. (B) Plates were scanned with automated ImmunoSpot analyzer. Bar graph of quantified IFN-gamma spots was plotted using GraphPad Prism v10. Results representative of four independent experiments.

FIG. 19A-F. Specificity of 237 CAR-T and TNGK CAR-T to Tn-expressing murine and human cell lines, respectively. (A) 237 and (B) TNGK murine CAR-T cells at 72 hours post-transduction (with 42.7% and 43.5% transduction efficiency, respectively), were co-cultured with (C) murine (ID8Cosmc-KO) and human ((D) Jurkat, (E) HEC151, and (F) MFE280) cell lines in 96-well plates at various tumor:effector ratio. Mock T cells and no T cells (tumor cells only) were included as negative controls. Cell culture supernatants harvested 24 hours after co-culture were analyzed for the murine cytokine IL-2 using ELISA assay. Plots were generated using GraphPad Prism v10. Results representative of at least three independent experiments. For (C)-(F), Open circle=mock T, closed circle=237 CAR T, closed square=TNGK CAR T; open square=tumor cells only.

FIG. 20A-F. LGQ scFv tetramer staining by flow cytometry: Jurkat and LOX-IMVI are promising human CAR-T targets. LGQ CAR, a derivative of 237 CAR, was purified as a single-chain variable fragment (scFv) fusion protein, biotinylated, and subsequently prepared as a tetramer staining reagent conjugated to Streptavidin-AlexaFluor647 (SA-AF647) for flow cytometry assays. Various human cell lines ((A) Jurkat, (B) A172, (C) LOX-IMVI, (D) VCaP, and (E) HCC2998) with predicted mutations or deep deletions of the CIGALTIC1 (COSMC) gene were stained either with the tetramer or a stepwise staining of LGQ scFv monomer followed by SA-AF647 secondary antibody. (F) Jurkat COSMC-KI (CIGALTIC1 gene knocked-in or overexpressed in Jurkat) was included as negative control. Both Jurkat and LOX-IMVI exhibited strong Tn-expression on their surface, while A172 and HCC2998 cell lines exhibited moderate Tn expression, and VCaP tested negative for Tn expression. (G) PAC-1 IC₅₀ at 72 hrs for the three solid tumor cell lines LOX-IMVI, HCC1998, and A172 were measured using the SRB assay and all three cell lines were found sensitive to PAC-1.

FIG. 21A-D. Co-culture of TNGK or LGQ CAR-T with human cell lines: IL-2 ELISA as readout. TNGK and LGQ murine CAR-T cells were co-cultured with various human cell lines (A) Jurkat, (B) LOX-IMVI, (C) Jurkat COSMC-KI, and (D) VCaP cell lines in 96-well plates at increasing tumor:effector ratio. Mock T cells and no T cells (tumor cells only) were included as negative controls. Cell culture supernatants harvested 24 hours after co-culture were analyzed for the murine cytokine IL-2 using ELISA assay. Plots were generated using GraphPad Prism v10. Results representative of at least three independent experiments.

FIG. 22A-C. Jurkat strongly express Tn antigen and are recognized by CAR-Ts. (A) Jurkat (top) and Jurkat COSMC-KI (bottom) cell lines were stained sequentially with the anti-Tn IgM antibody, followed by anti-IgM conjugated to AlexaFluor647 (AF647) and analyzed by Flow cytometry. Cell culture supernatants from either Jurkat (top) or Jurkat COSMC-KI (bottom) cell lines co-cultured with TNGK CAR-T or LGQ CAR-T or mock T or no T cells at varying tumor:effector ratio for 24 hours were analyzed for murine cytokine IL-2 expression by ELISA. (B) Jurkat (top) and Jurkat COSMC-KI tumors (bottom) grown subcutaneously in athymic nude mice were excised and fixed in 10% formalin for 24 hours followed by 70% ethanol. Tissue sections from formalin-fixed paraffin embedded (FFPE) samples were stained for Tn expression using purified biotinylated anti-Tn IgM antibody.

FIG. 23A-B. Tn expression in FFPE Jurkat tumors assessed by immunohistochemistry (IHC). Biotinylated anti-Tn IgM and biotinylated LGQ scFv fusion protein were both tested against Jurkat FFPE tissue sections (top panel) as IHC staining reagents for detection of Tn expression on cell membranes. Staining of Jurkat COSMC-KI FFPE tissue sections (bottom panel) served as negative control for both staining reagents.

### DETAILED DESCRIPTION

The present invention relates generally to the compositions and methods of use of T-cells genetically modified to stably express a desired chimeric antigen receptor (CAR) in conjunction with the procaspase activator PAC-1, to treat, for example, certain cancers, and in particular, certain solid tumor cancers. T-cells expressing a CAR are referred to herein as CAR T-cells or CAR modified T-cells. Preferably, the T-cell can be genetically modified to stably express an antibody binding domain on its surface, conferring novel antigen specificity that is MHC independent. In some instances, the T-cell is genetically modified to stably express a CAR that combines an antigen recognition domain of a specific antibody with an intracellular domain of, for example, the CD3-zeta chain or FcγRI protein into a single chimeric protein.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, cell culturing, etc.) are known in the art and described, for example, in Molecular Cloning: A Laboratory Manual, J. Sambrook et al., 4th edition, Cold Spring Harbor Laboratory Press, 2012; also see the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units is also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number 10”, it implies a continuous range that includes whole numbers and fractional numbers less than number 10, as discussed above. Similarly, if the variable disclosed is a number greater than “number 10”, it implies a continuous range that includes whole numbers and fractional numbers greater than number 10. These ranges can be modified by the term “about”, whose meaning has been described above.

One skilled in the art will also readily recognize that where members are grouped together in a common manner, such as in a Markush group, the disclosure encompasses not only the entire group listed as a whole, but each member of the group individually and all possible subgroups of the main group. Additionally, for all purposes, the disclosure encompasses not only the main group, but also the main group absent one or more of the group members. The disclosure therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping, or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, a patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

As used herein, the terms “providing”, “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject. The compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

As used herein, the terms “selectively binds to” or “preferentially binds to” mean that the compound, peptide or peptidomimetic, or other agent binds to the indicated molecule(s) or class of molecules with a higher affinity (e.g., at least 10-fold, in certain aspects of the disclosure: 100-fold) compared to a reference molecule.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, this disclosure also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Nucleic acid sequences cited herein are written in a 5′ to 3′ direction unless indicated otherwise. The term “nucleic acid” refers to either DNA or RNA or a modified form thereof comprising the purine or pyrimidine bases present in DNA (adenine “A”, cytosine “C”, guanine “G”, thymine “T”) or in RNA (adenine “A”, cytosine “C”, guanine “G”, uracil “U”). Interfering RNAs provided herein may comprise “T” bases, for example at 3′ ends, even though “T” bases do not naturally occur in RNA. In some cases, these bases may appear as “dT” to differentiate deoxyribonucleotides present in a chain of ribonucleotides.

The term “polynucleotide” refers to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, or EST), exons, introns, messenger RNA (mRNA), transfer RNA, ribosomal RNA, ribozymes, cDNA, RNAi, siRNA, recombinant polynucleotides, branched polynucleotides, plasmids, vectors, isolated DNA of any sequence, isolated RNA of any sequence, nucleic acid probes and primers. A polynucleotide can comprise, or alternatively consist essentially of, or yet further consist of modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment described herein that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

A “viral vector” is defined as a recombinantly produced virus or viral particle that comprises, or alternatively consists essentially of, or yet further consists of a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5 (7): 823-827. In aspects where gene transfer is mediated by a retroviral vector, a vector construct refers to the polynucleotide comprising, or alternatively consisting essentially of, or yet further consisting of the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising, or alternatively consisting essentially of, or yet further consisting of the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat et al., (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

As used herein, the term “fusion polypeptide” refers to proteins or polypeptides created through the joining of two or more genes or portion of genes that originally coded for separate polypeptides.

The term “immunotherapy” refers to the treatment of disease by activating or suppressing the immune system with, for example, an “immunotherapeutic”. Immunotherapies designed to elicit or amplify an immune response are classified as activation immunotherapies, while immunotherapies that reduce or suppress are classified as suppression immunotherapies. Immunotherapy is the treatment of disease by activating or suppressing the immune system. These immunotherapies are designed to elicit or amplify an immune response are classified as activation immunotherapies, while immunotherapies that reduce or suppress are classified as suppression immunotherapies. Cancer immunotherapy attempts to stimulate the immune system to destroy tumors.

### Embodiments of the Disclosure

In some embodiments, the present disclosure provides a composition comprising: (a) a procaspase-3 activator PAC-1, (PAC-1); and (b) a population of T-cells comprising a chimeric antigen receptor (CAR) fusion protein displayed on a cell surface membrane of the T-cells, wherein the CAR fusion protein selectively binds to an N-acetylgalactosamine-O-Serine or N-acetylgalactosamine-O-Threonine antigen moiety (Tn-antigen) of a protein disposed on a surface of a tumor cell. Optionally, a composition may include a pharmaceutically acceptable excipient or carrier.

In various additional embodiments, the procaspase-3 activator is a compound disclosed in U.S. Pat. Nos. 8,592,584; 8,778,945; 8,916,705; or 9,249,116; the formulas and compounds of which are incorporated herein by reference. The structure of PAC-1 is shown below:

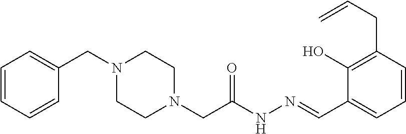 [SMILES: C=CCc1cccc(/C=N/NC(=O)CN2CCN(Cc3ccccc3)CC2)c1O]

In some embodiments, the CAR comprises a fusion protein comprising an extracellular domain having an antigen recognition domain, a transmembrane domain, and a cytoplasmic domain. In one embodiment, the transmembrane domain that naturally is associated with one of the domains in the CAR is used. In another embodiment, the transmembrane domain can be selected or modified by amino acid substitution to avoid binding of such domains to the transmembrane domains of the same or different surface membrane proteins to minimize interactions with other members of the receptor complex.

In some embodiments of the CAR fusion protein, the antibody moiety or antibody recognition domain comprises a single chain variable fragment (scFv), a single domain antibody, a bispecific antibody, or a multispecific antibody.

The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen, which is sometimes referred to herein as the antigen binding domain. The constant regions of the antibodies may mediate the binding of the immunoglobulin to host tissues or factors, including various cells of the immune system (e.g., effector cells) and the first component (Clq) of the classical complement system.

The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies described herein), single chain variable fragments, and single domain antibodies. The antibodies can be of any isotype/class (e.g., IgG, IgE, IgM, IgD, IgA and IgY) or subclass (e.g., IgG1, IgG2, IgG3, IgG4, IgAQ1 and IgA2). Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively. Examples of antibodies and methods of preparing the same are described, for example, in U.S. Pat. Nos. 4,816,567; 5,789,215; 6,596,541; 7,582,298; and 8,502,018.

In some embodiments, the antibody moiety or antigen recognition domain is a scFv. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}. ScFv molecules are known in the art and their production is described, for example, in U.S. Pat. Nos. 4,946,778 and 5,641,870.

In other embodiments, the antibody moiety is a bispecific antibody. The term “bispecific antibody” refers to an antibody that shows specificities to two different types of antigens. In other embodiments, the first antibody binding moiety and/or the second antibody binding moiety are multispecific antibodies. The term “multispecific antibody” as used herein refers to a molecule that binds to two or more different epitopes on one antigen or on two or more different antigens. Recognition of each antigen is generally accomplished with an “antigen binding domain”. The multispecific antibody may include one polypeptide chain that comprises a plurality, e.g., two or more, e.g., two, antigen binding domains. In some embodiments, the multispecific antibody may include two, three, four or more polypeptide chains that together comprise a plurality, e.g., two or more, e.g., two, antigen binding domains. Examples of the production and isolation of bispecific and multispecific antibodies are described in, for example, PCT Patent Publications WO 2014/031174 and WO 2009/080252.

Also contemplated are antibody moieties comprising single domain antibodies. The term “single domain antibodies” refers to the variable regions of either the heavy (VH) or light (VL) chain of an antibody. Single domain antibodies are described, for example in U.S. Patent Pub. No. 2006/0002935.

In certain embodiments, the scFv molecules may be produced from cDNA molecules or other polynucleotides encoding the variable regions of the heavy and light chains of the mAb that may be amplified by standard polymerase chain reaction (PCR) methodology using a set of primers for immunoglobulin heavy and light variable regions (Clackson (1991) Nature, 352, 624-628) (Also see U.S. Pat. No. 6,287,569). The amplified cDNAs encoding mAb heavy and light chain variable regions then may be linked together with a linker polypeptide in order to generate a recombinant scFv DNA molecule. Other polynucleotide elements may be included in the recombinant fusion protein such as an epitope tag and/or another protein that may anchor the scFv molecule on the surface of the exosome. In certain embodiments, the scFv molecules are genetically fused to the polynucleotide sequence of, for example, one or more epitope tags, affinity tags, solubility enhancing tags, and the like. Examples of various additional tags and linkers that may be used with aspects of the present disclosure include, haemagglutinin (HA) epitope, myc epitope, histidine tag, chitin binding protein (CBP), maltose binding protein (MBP), glutathione-S-transferase (GST), calmodulin binding peptide, biotin carboxyl carrier protein (BCCP), FLAG octapeptide, nus, green fluorescent protein (GFP), thioredoxin, poly (NANP), V5, S-protein, streptavidin, SBP, poly(Arg), DsbA, c-myc-tag, HAT, cellulose binding domain, softag 1, softag3, small ubiquitin-like modifier (SUMO), and ubiquitin (Lib). In certain embodiments, the fusion protein includes an epitope tag at the n-terminus or the c-terminus of the fusion protein. In preferred embodiments, the epitope tag is a hemagglutinin (HA) epitope tag disposed at the N-terminus of the fusion protein, and/or a 6× Histidine tag at the C-terminal end of the fusion protein.

In some embodiments, the antigen recognition domain is connected to the transmembrane region (or domain) by a hinge region (or domain). In some embodiments, the hinge region comprises an IgG-based hinge, or a membrane proximal region from CD8 and/or CD28, or a portion thereof. In some embodiments, the hinge region comprises a CD8 membrane-proximal region.

In some embodiments of a CAR fusion protein comprise a cytoplasmic domain or cell-signaling domain. Exemplary cytoplasmic domains include, but are not limited to, CD3ζ, CD3ζ (EBV), CD3ζ (Influenza MP-1), CD3ζ (VSV), CD3ε, 4-1BB, CD28, FCεRIγ, FcεRIγ (alloantigen), 4-1BB-CD3ζ, CD28-CD3ζ, CD28-CD3ζ (EBV), CD28-CD3ζ (Influenza), CD4-CD3ζ, CD28-4-1BB, CD4-FCεRIγ, CD28-FcεRIγ, CD28-41BB-CD3ζ, and CD28-OX40-CD3ζ. In preferred embodiments, the cytoplasmic domain comprises CD28/CD3ζ cytoplasmic domains.

In some embodiments, a CAR fusion protein comprises a transmembrane domain disposed between the antigen recognition moiety and the cytoplasmic domain. In some embodiments, the transmembrane region comprises a CD28 transmembrane region, an ICOS transmembrane region, 4-1BB, or OX-40. In some embodiments, a transmembrane region of a CAR fusion protein comprises the CD28 transmembrane region, and further comprises 4-1BB, OX-40, or Lck.

In some embodiments, the antibody moiety of the CAR fusion protein specifically binds to one or more N-acetylgalactosamine-O-Serine or N-acetylgalactosamine-O-Threonine antigen moiety (Tn-antigen) that is part of a protein disposed on a surface of a cancer cell. Tn-antigens generally denotes GalNAca-O-Ser/Thr, i.e., an antigen wherein the GalNAc residue is alpha-linked directly to the hydroxyl group of a serine or threonine residue of a polypeptide chain expressed intracellularly or at the cell surface. In some embodiments, the protein on the surface of the tumor cell expressing the Tn-antigen moiety comprises one or more of CD19 (Tn-CD19), BCMA (Tn-BCMA), CD22 (Tn-CD22), CD30 (Tn-CD30), CD7 (Tn-CD7), CD38 (Tn-CD38), kappa light chain (Tn-kappa light chain), CD20 (Tn-CD20), GD2 (Tn-GD2), HER2 (Tn-HER2), IL-13Rα2 (Tn-IL-13Rα2), EGFR (Tn-EGFR), mesothelin (Tn-Mesothelin), claudin-18.2 (Tn-claudin-18.2), PSMA (Tn-PMSA), OTS8 (Tn-OTS8), and MUC1 (Tn-MUC1), wherein each of the proteins expresses a Tn-antigen moiety. In other embodiments, the Tn-antigen comprises one or more of folate receptor alpha (Tn-), mesothelin (Tn-Mesothelin), B7-H3 (Tn-B7-H3), TAG72 (Tn-TAG72), MUC16 (Tn-MUC16), placental alkaline phosphatase (Tn-PAP), OTS8 (Tn-OTS8), and MUC1 (Tn-MUC1). In some preferred embodiments, the protein on the surface of the tumor cell comprising the Tn-antigen moiety is OTS8 (Tn-OTS8) or MUC1 (Tn-MUC1).

In some embodiments, the CAR fusion protein comprises a formula A-B-C-D, wherein A is the scFv, B is the hinge region, C is the transmembrane domain, and D is the cytoplasmic domain. In some embodiments, the hinge region is derived from a CD8 protein, the transmembrane domain is derived from a CD28 protein, and the cytoplasmic domain is derived from a CD32 and/or CD28 protein.

In one embodiment, the 237 CAR fusion protein comprises the amino acid sequence

(SEQ ID NO: 1)
AAATMGWSCIILFLVATATGVHSDIQLTQSPLSLPVSLGDQASISCRSSQS
LVHSNGNTYLHWYLQKPGQSPKLLIYKVSNRFSGVPDRFSGSGSGTDFTLK
ISSVEAEDLGVYFCSQSTHVPTFGGGTKLEIKGGGGSGGGGSGGGGSQVQL
QQSGGGLVQPGGSMKIFCAASGFTFSDAWMDWVRQSPEKGLEWVAEIRNKA
NNHETYYAESVKGRFTITRDDSKSRMSLQMNSLRAEDTGIYYCSGGKVRNA
YWGQGTTVTVSSLEKVNSTTTKPVLRTPSPVHPTGTSQPQRPEDCRPRGSV
KGTGLDFACDSSPKLFWALVVVAGVLFCYGLLVTVALCVIWTNSRRNRLLQ
VTTMNMTPRRPGLTRKPYQPYAPARDFAAYRPAHARAKFSRSAETAANLQD
PNQLYNELNLGRREEYDVLEKKRARDPEMGGKQQRRRNPQEGVYNALQKDK
MAEAYSEIGTKGERRRGKGHDGLYQGLSTATKDTYDALHMQTLAPR*

In one embodiment, the WE CAR fusion protein comprises the amino acid sequence of:

(SEQ ID NO: 2)
AAATMGWSCIILFLVATATGVHSDIQLTQSPLSLPVSLGDQASISCRSSQS
LVHSNGNTYLHWYLQKPGQSPKLLIYKVSNRFSGVPDRFSGSGSGTDFTLK
ISSVEAEDLGVYFCSQSTWEPTFGGGTKLEIKGGGGSGGGGSGGGGSQVQL
QQSGGGLVQPGGSMKIFCAASGFTFSDAWMDWVRQSPEKGLEWVAEIRNKA
NNHETYYAESVKGRFTITRDDSKSRMSLQMNSLRAEDTGIYYCSGGKVRNA
YWGQGTTVTVSSLEKVNSTTTKPVLRTPSPVHPTGTSQPQRPEDCRPRGSV
KGTGLDFACDSSPKLFWALVVVAGVLFCYGLLVTVALCVIWTNSRRNRLLQ
VTTMNMTPRRPGLTRKPYQPYAPARDFAAYRPAHARAKFSRSAETAANLQD
PNQLYNELNLGRREEYDVLEKKRARDPEMGGKQQRRRNPQEGVYNALQKDK
MAEAYSEIGTKGERRRGKGHDGLYQGLSTATKDTYDALHMQTLAPR*

In one embodiment, the TNGK CAR fusion protein comprises the amino acid sequence of:

(SEQ ID NO: 3)
AAATMGWSCIILFLVATATGVHSDIQLTQSPLSLPVSLGDQASISCRSSQS
LVHSNGNTYLHWYLQKPGQSPKLLIYKVSNRFSGVPDRFSGSGSGTDFTLK
ISSVEAEDLGVYFCSQTNGKPTFGGGTKLEIKGGGGSGGGGSGGGGSQVQL
QQSGGGLVQPGGSMKIFCAASGFTFSDAWMDWVRQSPEKGLEWVAEIRNKA
NNHETYYAESVKGRFTITRDDSKSRMSLQMNSLRAEDTGIYYCSGGKVRNA
YWGQGTTVTVSSLEKVNSTTTKPVLRTPSPVHPTGTSQPQRPEDCRPRGSV
KGTGLDFACDSSPKLFWALVVVAGVLFCYGLLVTVALCVIWTNSRRNRLLQ
VTTMNMTPRRPGLTRKPYQPYAPARDFAAYRPAHARAKFSRSAETAANLQD
PNQLYNELNLGRREEYDVLEKKRARDPEMGGKQQRRRNPQEGVYNALQKDK
MAEAYSEIGTKGERRRGKGHDGLYQGLSTATKDTYDALHMQTLAPR*

In one embodiment, the LGQ CAR fusion protein comprises the amino acid sequence of:

(SEQ ID NO: 4)
AAATMGWSCIILFLVATATGVHSDIQLTQSPLSLPVSLGDQASISCRSSQS
LVHSNGNTYLHWYLQKPGQSPKLLIYKVSNRFSGVPDRFSGSGSGTDFTLK
ISSVEAEDLGVYFCSQSLGQPTFGGGTKLEIKGGGGSGGGGSGGGGSQVQL
QQSGGGLVQPGGSMKIFCAASGFTFSDAWMDWVRQSPEKGLEWVAEIRNKA
NNHETYYAESVKGRFTITRDDSKSRMSLQMNSLRAEDTGIYYCSGGKVRNA
YWGQGTTVTVSSLEKVNSTTTKPVLRTPSPVHPTGTSQPQRPEDCRPRGSV
KGTGLDFACDSSPKLFWALVVVAGVLFCYGLLVTVALCVIWTNSRRNRLLQ
VTTMNMTPRRPGLTRKPYQPYAPARDFAAYRPAHARAKFSRSAETAANLQD
PNQLYNELNLGRREEYDVLEKKRARDPEMGGKQQRRRNPQEGVYNALQKDK
MAEAYSEIGTKGERRRGKGHDGLYQGLSTATKDTYDALHMQTLAPR*

*Underlined=leader sequence; italicized=237 scFv region; bold=CD8 hinge region; italicized and underlined=CD28 region; and bold and italicized=CD3ζ region. The WE CAR fusion protein, TNGK CAR fusion protein, and the LGQ are derived from the 237 scFV region and include one or more amino acid changes within the region corresponding to positions 116-124 of the 237 scFv moiety (SEQ ID NO: 1) as described below:

| | |
|---|---|
| 237 CAR | SQSTHVPT (SEQ ID NO: 5) |
| WE CAR | SQSTWEPT (SEQ ID NO: 6) |
| TNGK CAR | SQTNGKPT (SEQ ID NO: 7) |
| LGQ CAR | SQSLGQPT (SEQ ID NO: 8) |

In some embodiments, the CAR fusion protein comprises an scFv moiety comprising SEQ ID NO: 5, SEQ ID NO: 6, SEQ ID NO: 7, or SEQ ID NO: 8.

In some embodiments, the CAR fusion protein is about 85% identical, about 86% identical, about 87% identical, about 88% identical, about 89% identical, about 90% identical, about 91% identical, about 92% identical, about 93% identical, about 94% identical, about 95% identical, about 96% identical, about 97% identical, about 98% identical, about 99% identical, or 100% identical to any one of SEQ ID NO: 1-8.

In certain embodiments, the subject CAR fusion proteins may be delivered via an expression construct—including a nucleic acid that provides a coding sequence for a fusion protein—to a target T-cell population in vitro where the transformed T-cell population expressing the CAR fusion protein is expanded, and collected for infusion into a host subject. For example, the expression construct can encode a CAR fusion protein that is displayed on a cell surface by the transduced cell. General laboratory techniques (DNA extraction, RNA extraction, cloning, cell culturing, etc.) are known in the art and described, for example, in Molecular Cloning: A Laboratory Manual, J. Sambrook et al., 4th edition, Cold Spring Harbor Laboratory Press, 2012.

As used herein, the term “nucleic acid” refers to polynucleotides such as deoxyribonucleic acid (DNA), and, where appropriate, ribonucleic acid (RNA). The term should also be understood to include, as appropriate to the context or as applicable to the embodiment being described, both single-stranded polynucleotides (such as antisense) and double-stranded polynucleotides (such as siRNAs).

In brief summary, the expression of natural or synthetic nucleic acids encoding CARs is typically achieved by operably linking a nucleic acid encoding the CAR fusion protein or portions thereof to a promoter, and incorporating the construct into an expression vector. The vectors can be suitable for replication and integration in eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Expression constructs of the disclosure may also be used for nucleic acid immunization and gene therapy, using standard gene delivery protocols. Methods for gene delivery are known in the art. See, e.g., U.S. Pat. Nos. 5,399,346, 5,580,859, and 5,589.466, incorporated by reference herein in their entireties. Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Methods of constructing CAR T-cells are known in the art are described, for example, in U.S. Pat. Nos. 9,540,445 and 9,499,629 to June et al., and U.S. Patent Publication No. 2018/0170992 to Balyasnikova et al. and generally following the following procedure: Isolate T cells using positive or negative selection, activate T cells with CD3/CD28 or similar antibody beads, transduce activated T cells using a retrovirus or lentivirus (for murine and human T, respectively) carrying the recombinant CARs, expand the recombinant CAR Ts with IL-2, and administer the CAR T cells to a subject.

Prior to expansion and genetic modification of the T-cells, a source of T-cells is obtained from a subject. T-cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments, any number of T-cell lines available in the art may be used. In certain embodiments, T-cells can be obtained from a unit of blood collected from a subject using any number of techniques known to the skilled artisan, such as Ficoll™ separation.

In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer additives such as 0.5% bovine serum albumin and 2 mM EDTA. Alternatively, the undesirable components of the apheresis sample may be removed, and the cells directly resuspended in culture media.

In another embodiment, T-cells are isolated from peripheral blood lymphocytes by lysing the red blood cells and depleting the monocytes, for example, by centrifugation through a PERCOLL™ gradient or by counterflow centrifugal elutriation. A specific subpopulation of T-cells, such as CD3⁺, CD28⁺, CD4⁺, CD8⁺, CD45RA⁺, and CD45RO⁺ T-cells, can be further isolated by positive or negative selection techniques. For example, in one embodiment, T-cells are isolated by incubation with anti-CD3/anti-CD28 (i.e., 3×28)-conjugated beads, such as DYNABEADS® M-450 CD3/CD28 T, for a time period sufficient for positive selection of the desired T-cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T-cells from patients with leukemia, use of longer incubation times, such as 24 hours, can increase cell yield. Longer incubation times may be used to isolate T-cells in any situation where there are few T-cells as compared to other cell types, such in isolating tumor infiltrating lymphocytes (TIL) from tumor tissue or from immune-compromised individuals. Further, use of longer incubation times can increase the efficiency of capture of CD8+ T-cells. Thus, by simply shortening or lengthening the time T-cells are allowed to bind to the CD3/CD28 beads and/or by increasing or decreasing the ratio of beads to T-cells (as described further herein), subpopulations of T-cells can be preferentially selected for or against at culture initiation or at other time points during the process. Additionally, by increasing or decreasing the ratio of anti-CD3 and/or anti-CD28 antibodies on the beads or other surface, subpopulations of T-cells can be preferentially selected for or against at culture initiation or at other desired time points. The skilled artisan would recognize that multiple rounds of selection can also be used in the context of this disclosure. In certain embodiments, it may be desirable to perform the selection procedure and use the “unselected” cells in the activation and expansion process. “Unselected” cells can also be subjected to further rounds of selection.

Enrichment of a T-cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich CD4⁺ cells by negative selection, a monoclonal antibody cocktail typically includes antibodies to CD 14, CD20, CD11b, CD 16, HLA-DR, and CD8. In certain embodiments, it may be desirable to enrich for or positively select for regulatory T-cells which typically express CD4⁺, CD25⁺, CD62L^{hi}, GITR⁺, and FoxP3⁺. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T-cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T-cells that normally have weaker CD28 expression.

In some embodiments, it may be desirable to use lower concentrations of cells. By significantly diluting the mixture of T-cells and surface (e.g., particles such as beads), interactions between the particles and cells are minimized. This selects for cells that express high amounts of desired antigens to be bound to the particles. For example, CD4⁺ T-cells express higher levels of CD28 and are more efficiently captured than CD8⁺ T-cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T-cells for stimulation can also be frozen after a washing step. Without being bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present disclosure.

Also contemplated in the context of the disclosure is the collection of blood samples or apheresis product from a subject at a time period prior to when the expanded cells as described herein might be needed. As such, the source of the cells to be expanded can be collected at any time point necessary, and desired cells, such as T-cells, isolated and frozen for later use in T-cell therapy for any number of diseases or conditions that would benefit from T-cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T-cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further embodiment, T-cells are obtained from a patient directly following treatment. In this regard, it has been observed that following certain cancer treatments, in particular treatments with drugs that damage the immune system, shortly after treatment during the period when patients would normally be recovering from the treatment, the quality of T-cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T-cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T-cells, B-cells, dendritic cells, and other cells of the immune system.

Whether prior to or after genetic modification of the T-cells to express a desirable CAR, the T-cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Publication No. 2006/0121005.

Generally, the T-cells are expanded by contact with a surface having attached thereto an agent that stimulates a CD3/TCR complex associated signal and a ligand that stimulates a co-stimulatory molecule on the surface of the T-cells. In particular, T-cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T-cells, a ligand that binds the accessory molecule is used. For example, a population of T-cells can be contacted with an anti-CD3 antibody and an anti-CD28 antibody, under conditions appropriate for stimulating proliferation of the T-cells. To stimulate proliferation of either CD4⁺ T-cells or CD8⁺ T-cells, an anti-CD3 antibody and an anti-CD28 antibody. Examples of an anti-CD28 antibody include 9.3, B-T3, XR-CD28 (Diaclone, Besançon, France) can be used as can other methods commonly known in the art (Berg et al., Transplant Proc. 30 (8): 3975-3977, 1998; Haanen et al., J. Exp. Med. 190(9):13191328, 1999; Garland et al., J. Immunol. Meth. 227(1-2):53-63, 1999).

In certain embodiments, the primary stimulatory signal and the co-stimulatory signal for the T-cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 2004/0101519 and 2006/0034810 for artificial antigen presenting cells (aAPCs) that are contemplated for use in activating and expanding T-cells in the present disclosure.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4⁺ T-cell expansion and T-cell growth is used. In certain aspects of the disclosure, a ratio of anti CD3:CD28 antibodies bound to the beads is used such that an increase in T-cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect, more anti-CD28 antibody is bound to the particles than anti-CD3 antibody, i.e., the ratio of CD3:CD28 is less than one. In certain embodiments, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T-cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T-cells.

In further embodiments, the cells, such as T-cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T-cells. In one embodiment the cells (for example, 10⁴ to 10⁹ T-cells) and beads (for example, DYNABEADS® M-450 CD3/CD28 T paramagnetic beads at a ratio of 1:1) are combined in a buffer, preferably PBS (without divalent cations such as calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present disclosure. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T-cells. Such populations of cells may have therapeutic value and would be desirable to obtain in certain embodiments. For example, using high concentration cells allows more efficient selection of CD8+ T-cells that normally have weaker CD28 expression.

In some embodiments, the disclosed composition comprises a pharmaceutically acceptable diluent, excipient, carrier, or a combination thereof. In other embodiments of the disclosed composition, a) the carrier comprises water, a buffer, a sugar, a cellulose, a cyclodextrin, dimethyl sulfoxide, polyethylene glycol, tocopherol, a liposome, a micelle, or a combination thereof, or b) the excipient comprises, a binder, a lubricant, a sorbent, a vehicle, a disintegrant, a preservative, or a combination thereof.

In various other embodiments, the concentration of PAC-1 in a composition is about 0.1 μM to about 50 μM. In other embodiments, the concentration of PAC-1 is about 0.1 μM to about 1 μM, about 1 μM to about 10 μM, about 2 μM to about 15 μM, about 3 μM to about 20 μM, about 4 μM to about 25 μM, about 5 μM to about 30 μM, about 10 μM to about 40 μM, about 15 μM to about 50 μM, or about 0.01 μM to about 100 μM.

In some embodiments, the amount of CAR T-cells in a composition comprises about 5×10⁵ T-cells to about 5×10⁸ T-cells, or about 5×10⁶ T-cells. In some embodiments, a dose of infused CAR T-cells comprises about 5×10⁵ T-cells to about 5×10⁸ T-cells, or about 5×10⁶ T-cells per kilogram of body weight. In some embodiments, the population of T-cells that express the CAR fusion protein is greater than about 10% of the T-cells of a given population, greater than about 15% of the T-cells of a given population, greater than about 20% of the T-cells of a given population, greater than about 25% of the T-cells of a given population, greater than about 30% of the T-cells of a given population, greater than about 35% of the T-cells of a given population, greater than about 40% of the T-cells of a given population, greater than about 45% of the T-cells of a given population, or greater than about 50% of the T-cells of a given population.

In further embodiments, the CAR T-cells and compositions thereof disclosed herein selectively targets cancer cells, wherein the cancer cells are cells of embryonal tumor, esophagogastric cancer, central nervous system cancer, bladder cancer, breast cancer, glioblastoma, head and neck cancer, hepatobiliary cancer, lung cancer, melanoma, meningioma, multiple myeloma, ocular melanoma, ovarian cancer, ovarian epithelial cancer, osteosarcoma, pancreatic cancer, prostate cancer, renal cancer, salivary gland cancer, sarcoma, seminoma, or soft tissue sarcoma. In some embodiments, the cancer is ovarian cancer. In other embodiments, the ovarian cancer is stage I ovarian cancer, stage II ovarian cancer, stage III ovarian cancer, or stage IV ovarian cancer. Ovarian cancer staging is described, for example, in Prat et al., (2014), International Journal of Gynecology & Obstetrics, 124:1-5. In some embodiments, the cancer comprises a solid tumor such as carcinoma, sarcoma, and lymphomas. In still other embodiments, the cancer comprises one or more of bladder cancer, soft tissue sarcoma, non-small cell lung cancer, lung cancer, esophagogastric cancer, and ovarian cancer,

In other embodiments, a cancer is one or more of Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma, Primary CNS Lymphoma, Anal Cancer, Astrocytomas, Atypical Teratoid/Rhabdoid Tumor, Central Nervous System Cancer, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Carcinoid Tumor (Gastrointestinal), Carcinoma of Unknown Primary, Cardiac (Heart) Tumors, Embryonal Tumors, Primary CNS Lymphoma, Cervical Cancer, Chordoma, Chronic Lymphocytic Leukemia (CLL), Chronic Myelogenous Leukemia (CML), Chronic Myeloproliferative Neoplasms, Colorectal Cancer, Craniopharyngioma, Endometrial Cancer (Uterine Cancer), Esophageal Cancer, Esthesioneuroblastoma, Ewing Sarcoma (Bone Cancer), Extracranial Germ Cell Tumor, Eye Cancer, Intraocular Melanoma, Retinoblastoma, Fallopian Tube Cancer, Fibrous Histiocytoma of Bone, Gallbladder Cancer, Gastric (Stomach) Cancer, Gastrointestinal Carcinoid Tumor, Gastrointestinal Stromal Tumors (GIST) (Soft Tissue Sarcoma), Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Langerhans Cell Histiocytosis, Hodgkin Lymphoma, Hypopharyngeal Cancer, Intraocular Melanoma, Islet Cell Tumors, Pancreatic Neuroendocrine Tumors, Kidney (Renal Cell) Cancer, Laryngeal Cancer, Leukemia, Lip and Oral Cavity Cancer, Liver Cancer, Lung Cancer (Non-Small Cell and Small Cell), Lymphoma, Male Breast Cancer, Malignant Fibrous Histiocytoma of Bone and Osteosarcoma, Melanoma, Merkel Cell Carcinoma, Mesothelioma, Metastatic Cancer, Metastatic Squamous Neck Cancer with Occult Primary, Midline Tract Carcinoma With NUT Gene Changes, Mouth Cancer, Multiple Endocrine Neoplasia Syndromes, Multiple Myeloma/Plasma Cell Neoplasms, Mycosis Fungoides, Myelodysplastic Syndromes, Myelodysplastic/Myeloproliferative Neoplasms, Chronic Myeloproliferative Neoplasms, Nasal Cavity and Paranasal Sinus Cancer, Nasopharyngeal Cancer, Neuroblastoma, Non-Hodgkin Lymphoma, Oral Cancer, Lip and Oral Cavity Cancer, Oropharyngeal Cancer, Osteosarcoma, Malignant Fibrous Histiocytoma of Bone, Ovarian Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Salivary Gland Cancer, Sarcoma, Soft Tissue Sarcoma, Uterine Sarcoma, Sézary Syndrome (Lymphoma), Skin Cancer, Small Intestine Cancer, Squamous Neck Cancer with Occult Primary, Metastatic, Stomach (Gastric) Cancer, T-Cell Lymphoma, Testicular Cancer, Throat Cancer, Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter, Ureter and Renal Pelvis Cancers, Transitional Cell Cancer (Kidney (Renal Cell) Cancer, Urethral Cancer, Uterine Cancer, Endometrial Uterine Sarcoma, Vaginal Cancer, Vascular Tumors, Vulvar Cancer, or Wilms Tumor.

Additionally, this disclosure provides a method of treating a cancer comprising administering to a subject in need thereof, concurrently or sequentially, a therapeutically effective amount of PAC-1 and a therapeutically effective amount of a population of T-cells comprising a chimeric antigen receptor (CAR) fusion protein displayed on a cell surface membrane the T-cells, wherein the effect of the T-cells is enhanced by the administration of the PAC-1. In some embodiments, the CAR fusion protein selectively binds to an N-acetylgalactosamine-O-Serine or N-acetylgalactosamine-O-Threonine antigen moiety (Tn-antigen) of a protein disposed on a surface of a tumor cell.

In some various embodiments, the compound PAC-1 and the CAR T-cells are concurrently administered to the subject. In yet other embodiments, the compound PAC-1 and the CAR T-cells are sequentially administered to the subject. In additional embodiments, the compound PAC-1 is administered to the subject before the CAR T-cells. In further embodiments, the compound PAC-1 is administered to the subject after the CAR T-cells.

In some embodiments, the PAC-1 is administered in a dose comprising about 1 mg/kg to about 200 mg/kg, about 1 mg/kg to about 175 mg/kg, about 1 mg/kg to about 150 mg/kg, about 1 mg/kg to about 125 mg/kg, about 1 mg/kg to about 100 mg/kg, about 1 mg/kg to about 75 mg/kg, about 1 mg/kg to about 50 mg/kg, about 1 mg/kg to about 25 mg/kg, about 1 mg/kg to about 20 mg/kg, about 1 mg/kg to about 15 mg/kg, or about 1 mg/kg to about 10 mg/kg. Dosage conversion between animals and humans are known in the art; see, for example, Nair et al, J Basic Clin Pharma 2016; 7:27-31.

In some embodiments, the population of CAR T-cells are administered in a dose of about 5×10⁵ T-cells to about 5×10⁸ T-cells, or about 5×10⁶ T-cells. In some embodiments, a dose of infused CAR T-cells comprises about 5×10⁵ T-cells to about 5×10⁸ T-cells, or about 5×10⁶ T-cells per kilogram of body weight.

In some embodiments, the population of T-cells that express the CAR fusion protein is greater than about 10% of the T-cells of a given population, greater than about 15% of the T-cells of a given population, greater than about 20% of the T-cells of a given population, greater than about 25% of the T-cells of a given population, greater than about 30% of the T-cells of a given population, greater than about 35% of the T-cells of a given population, greater than about 40% of the T-cells of a given population, greater than about 45% of the T-cells of a given population, greater than about 50% of the T-cells of a given population, greater than about 55% of the T-cells of a given population, or greater than about 60% of the T-cells of a given population. In other embodiments, a population of CAR T-cell comprises a mixture of the CAR T cells and non-transformed T cells in a ratio selected from about 1:5 of the CAR T cells to the non-transformed T cells, about 1:4 of the CAR T cells to the non-transformed T cells, about 1:3 of the CART cells to the non-transformed T cells, about 1:2 of the CAR T cells to the non-transformed T cells, and about 1:1 of the CAR T cells to the non-transformed T cells.

In some embodiments, the PAC-1 is administered in a dose comprising about 10 mg/kg to about 125 mg/kg; and/or the population of T-cells are administered in a dose comprising about 5×10⁵ T-cells to about 5×10⁸ T-cells.

In other embodiments, the compound PAC-1 is administered to the subject once daily (q.d.), twice a day (b.i.d.), three times a day (t.i.d.), or four times a day (q.i.d.), wherein the total administered dose per day of PAC-1 is about 1 mg/kg to about 150 mg/kg; or each administered dose of PAC-1 is about 70 mg, about 175 mg, about 250 mg, about 375 mg, about 450 mg, about 500 mg, about 625 mg, about 750 mg, or about 800 mg; or each administered dose of PAC-1 is about 50 mg/m² to about 250 mg/m². In some embodiments, the amount of PAC-1 administered to a subject does not exceed 750 mg/day.

In other embodiments, the compound PAC-1 is administered over the course of three administration time periods, wherein each of the administration time periods is 5 consecutive days with at least two days between each of the administration time periods, wherein the PAC-1 is administered once daily for each of the 5 consecutive days; and the population of T-cells are administered during one of a second or third administration time period. Various form of administration of the CAR T-cells and PAC-1 compound are contemplated, including oral, sublingual and buccal, rectal, parenteral routes of administration such as subcutaneous, intramuscular, intravenous, intraperitoneal, and intrathecal, and other routes of administration such as topical, ocular, otic, intranasal, and vaginal.

In one certain embodiment, a method of treating ovarian cancer comprising administering to a subject in need thereof, concurrently or sequentially, a therapeutically effective amount of: PAC-1; and a population of T-cells comprising a chimeric antigen receptor (CAR) fusion protein displayed on a cell surface membrane of a T-cell, wherein the CAR fusion protein comprises one or more of a WE single chain variable fragment (scFv), a 237 single chain variable fragment (scFv), a LGQ single chain variable fragment (scFv), and a TNGK single chain variable fragment (scFv); wherein each of the WE scFv, 237 scFv, LGQ scFv, and the TNGK scFv, selectively bind to an N-acetylgalactosamine-O-Serine or N-acetylgalactosamine-O-Threonine antigen, thereby treating the ovarian cancer.

In one certain embodiment, a method of treating ovarian cancer comprising administering to a subject in need thereof, concurrently or sequentially, a therapeutically effective amount of: PAC-1; and a population of T-cells comprising a chimeric antigen receptor (CAR) fusion protein displayed on a cell surface membrane of a T-cell, wherein the CAR fusion protein comprises one or more of a WE single chain variable fragment (scFv) and a TNGK single chain variable fragment (scFv); wherein each of the WE scFv and the TNGK scFv, selectively bind to an N-acetylgalactosamine-O-Serine or N-acetylgalactosamine-O-Threonine antigen, thereby treating the ovarian cancer.

In another embodiment, a method of treating a solid tumor comprising administering to a subject in need thereof, concurrently or sequentially, a therapeutically effective amount of: PAC-1; and a population of T-cells comprising a chimeric antigen receptor (CAR) fusion protein displayed on a cell surface membrane of a T-cell, wherein the CAR fusion protein comprises one or more of a WE single chain variable fragment (scFv), a 237 single chain variable fragment (scFv), a LGQ single chain variable fragment (scFv), and a TNGK single chain variable fragment (scFv); wherein each of the WE scFv, the 237 scFv, the LGQ scFv, and the TNGK scFv, selectively bind to an N-acetylgalactosamine-O-Serine or N-acetylgalactosamine-O-Threonine antigen, thereby treating the solid tumor.

In another embodiment, a method of treating stage II or stage IV ovarian cancer comprises administering to a subject in need thereof, concurrently or sequentially, a therapeutically effective amount of: PAC-1; and a population of T-cells comprising a chimeric antigen receptor (CAR) fusion protein displayed on a cell surface membrane of a T-cell, wherein the CAR fusion protein comprises one or more of a WE single chain variable fragment (scFv), a 237 single chain variable fragment (scFv), a LGQ single chain variable fragment (scFv), and a TNGK single chain variable fragment (scFv); wherein each of the WE scFv, 237 scFv, LGQ scFv, and the TNGK scFv, selectively bind to an N-acetylgalactosamine-O-Serine or N-acetylgalactosamine-O-Threonine antigen, thereby treating the stage II or the stage IV ovarian cancer.

In some embodiments of the methods of treating cancer (e.g., ovarian cancer) or other solid tumors, the 237 single chain variable fragment (scFv) comprises the amino acid sequence set forth in SEQ ID NO: 1, the WE single chain variable fragment (scFv) comprises the amino acid sequence set forth in SEQ ID NO: 2, a TNGK single chain variable fragment (scFv) comprises the amino acid sequence set forth in SEQ ID NO: 3; and the LGQ single chain variable fragment (scFv) comprises the amino acid sequence set forth in SEQ ID NO: 4.

In another embodiment, a method of treating a cancer, wherein the cancer comprises N-acetylgalactosamine-O-Serine or N-acetylgalactosamine-O-Threonine antigen (Tn-antigen) expressing cancer cells, the method comprising administering to a subject in need thereof, concurrently or sequentially, a therapeutically effective amount of: PAC-1; and a population of T-cells comprising a chimeric antigen receptor (CAR) fusion protein displayed on a cell surface membrane of a T-cell, wherein the CAR fusion protein comprises one or more of a TNGK single chain variable fragment (scFv) (SEQ ID NO: 3), a WE single chain variable fragment (scFv) (SEQ ID NO: 2), a 237 single chain variable fragment (scFv) (SEQ ID NO: 1), and a LGQ single chain variable fragment (scFv) (SEQ ID NO: 4); and wherein each of the TNGK scFv, the WE scFv, the 237 scFv, and the LGQ scFv, selectively bind the Tn-antigen, thereby treating the cancer.

### Pharmaceutical Formulations.

The compounds and compositions described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium, or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical, or subcutaneous routes.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose, or aspartame; or a flavoring agent such as peppermint, oil of wintergreen, or cherry flavoring, may be added. When the unit dosage form is a capsule, it may contain, in addition to materials of the above type, a liquid carrier, such as vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), U.S. Pat. No. 4,820,508 (Wortzman), U.S. Pat. No. 4,608,392 (Jacquet et al.), and U.S. Pat. No. 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compositions described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the disclosure provides a composition comprising a compound of the disclosure formulated in such a unit dosage form.

The compound can be conveniently administered in a unit dosage form, for example, containing 5 to 800 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The compounds described herein can be effective anti-tumor agents and have higher potency and/or reduced toxicity as compared to immunotherapies alone or other cancer treatments.

The disclosure provides therapeutic methods of treating cancer in a mammal, which involve administering to a mammal having cancer an effective amount of a compound or composition described herein. A mammal includes a primate, human, rodent, canine, feline, bovine, ovine, equine, swine, caprine, bovine, vertebrates, and the like. Cancer refers to any various type of malignant neoplasm, for example, colon cancer, breast cancer, ovarian cancer, osteosarcoma, melanoma and leukemia, and in general is characterized by an undesirable cellular proliferation, e.g., unregulated growth, lack of differentiation, local tissue invasion, and metastasis.

The ability of a compound of the disclosure to treat cancer may be determined by using assays well known to the art. For example, the design of treatment protocols, toxicity evaluation, data analysis, quantification of tumor cell kills, and the biological significance of the use of transplantable tumor screens are known.

The following Examples are intended to illustrate the above disclosure and should not be construed as to narrow its scope. One skilled in the art will readily recognize that the Examples suggest many other ways in which the disclosure could be practiced. It should be understood that numerous variations and modifications may be made while remaining within the scope of the disclosure.

### EXAMPLES

### Example 1. Single CAR-T Cell Treatment Controls Disseminated Ovarian Cancer in a Syngeneic Mouse Model

Treatment of some blood cancers with T-cells that express a chimeric antigen receptor (CAR) against CD19 have shown remarkable results. In contrast, CAR-T cell efficacy against solid tumors has been difficult to achieve.

To examine the potential of CAR-T cell treatments against ovarian cancers, we used the mouse ovarian cancer cell line ID8 in an intraperitoneal model that exhibits disseminated solid tumors in female C57BL/6J mice. The CAR contained a scFv from antibody 237 which recognizes a Tn-glycopeptide-antigen expressed by ID8 due to aberrant O-linked glycosylation in the absence of the transferase-dependent chaperone Cosmc. The efficacy of four Tn-dependent CARs with varying affinity to Tn antigen, and each containing CD28/CD3ζ cytoplasmic domains, were compared in vitro and in vivo in this study.

In line with many observations about the impact of aberrant O-linked glycosylation, the ID8Cosmc knock-out (ID8Cosmc-KO) exhibited more rapid tumor progression compared to wild-type ID8. Despite the enhanced tumor growth in vivo, 237 CAR and a mutant with 30-fold higher affinity, but not CARs with lower affinity, controlled advanced ID8Cosmc-KO tumors. Tumor regression could be achieved with a single intravenous dose of the CARs, but intraperitoneal administration was even more effective. The CAR-T cells persisted over a period of months, allowing CAR-treated mice to delay tumor growth in a re-challenge setting. The most effective CARs exhibited the highest affinity for antigen. Anti-tumor effects observed in vivo were associated with increased numbers of T-cells and macrophages, and higher levels of cleaved caspase-3, in the tumor microenvironment. Notably, the least therapeutically effective CAR mediated tonic signaling leading to antigen-independent cytokine expression and it had higher levels of the immunosuppressive cytokine IL-10.

The findings support the development of affinity-optimized CAR-T cells as a potential treatment for established ovarian cancer, with the most effective CARs mediating a distinct pattern of inflammatory cytokine release in vitro. Importantly, the most potent Tn-dependent CAR-T cells showed no evidence of toxicity in tumor-bearing mice in a syngeneic, immunocompetent system.

Solid tumors have been difficult to treat with CAR-T cells because of a lack of suitable targets. Despite the tremendous promise of CAR-T cell therapy against solid tumors, a majority of pre-clinical studies with CARs have used human tumors in immune-deficient mouse models.

We established an immunocompetent, syngeneic murine model to characterize Tn-antigen specific CAR-T cells targeting advanced stages of ovarian solid tumors. We found evidence of direct CAR T-cell mediated cytotoxicity against tumor cells and exhibited long-term delay of tumor progression when administered to mice with disseminated tumors.

In this study, we developed guidelines for successful CAR-T cell treatment of advanced ovarian cancer. We demonstrate that a single dose of Tn-dependent CAR with sufficient affinity for a Tn-glycoprotein is able to control well-established tumors in this model, and was most effective with intraperitoneal administration. The study also provides additional evidence of the role of CAR affinity in the optimization of solid tumor targeting.

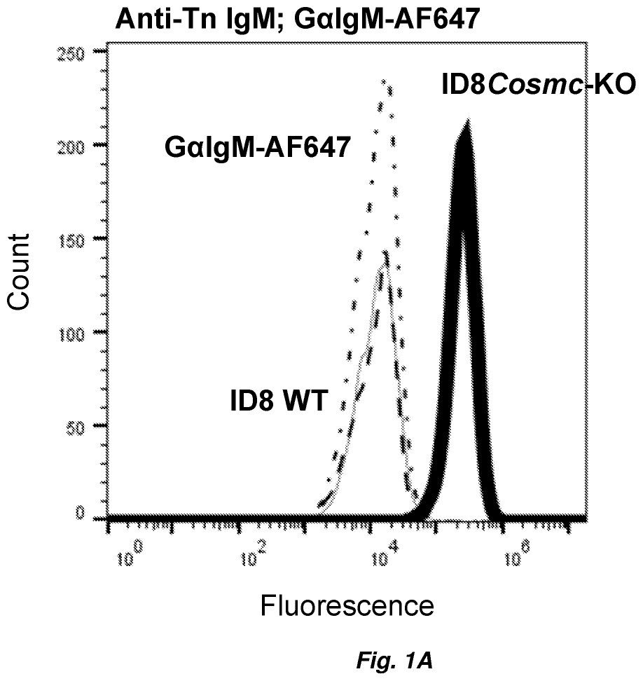

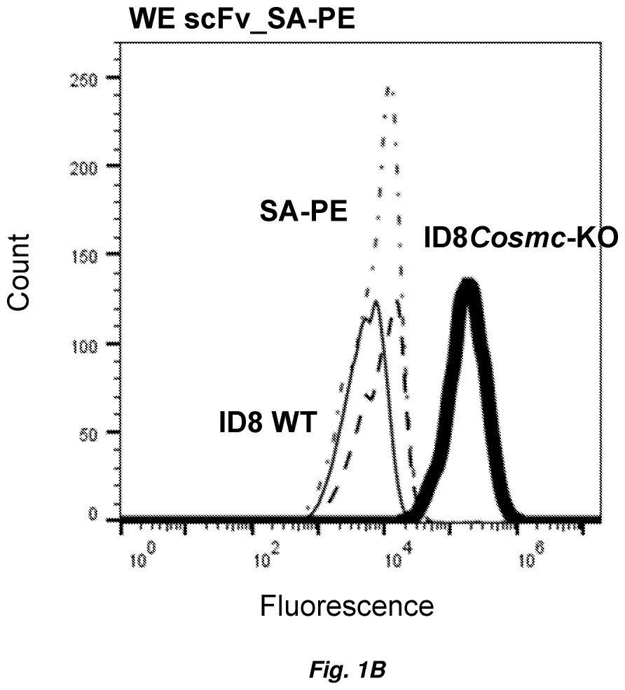

ID8Cosmc-KO as a model for CAR treatment of ovarian cancer. The ovarian cancer cell line ID8, with a deletion of the gene for the chaperone Cosmc, expresses high levels of Tn-antigen as detected with anti-Tn antibody 5F4 (FIG. 1A). ID8Cosmc-KO is also recognized by Tn-dependent antibody 237 and its CAR derivatives. For detection, the high-affinity variant called 237-WE (WE from here-on) was expressed as a soluble biotinylated scFv. Tetramers of the WE scFv were used to validate the expression of the Tn-OTS8-antigen in the ID8Cosmc-KO line (FIG. 1B). Notably, the parental ID8 (ID8 WT) and ID8Cosmc-KO lines exhibited the same rate of growth in vitro, with doubling times of 19.2 h and 21.8h, respectively (FIG. 1C).

To evaluate the ID8Cosmc-KO as a model for 237 CAR treatment, we inoculated 10⁷ tumor cells i.p. into syngeneic C57BL/6 mice. We used ID8 lines without luciferase for imaging to minimize the potential for immunogenicity of foreign proteins in immunocompetent mice. Consistent with previous ID8 studies, mice with ID8 WT began to develop ascites and gained weight to the point of euthanasia criterion (36 g or >50% weight gain) within 10 to 12 weeks. Mice with ID8Cosmc-KO tumors developed slightly earlier signs of ascites (8 weeks, and the median survival was 73 days, with 100% tumor take). By comparison, ID8 WT had a median survival of 88 days. To assess the possible role of B- or T-cells in ID8 Cosmc-KO growth, immunodeficient Rag1-KO mice were inoculated with 10⁷ ID8Cosmc-KO cells. Growth of the tumor was rapid, with a median survival of 28 days, indicating a significant contribution of acquired immune responses in immunocompetent mice. Mice showed signs of lethargy but no ascites-related weight gain.

At endpoint criterion, immunocompetent C57BL/6 mice exhibited extensive tumor growth distributed throughout the peritoneal cavity, typically with the presence of ascites fluid (median, 17 mL). Solid tumors were most often located in association with adipose tissues that surround organs. Pathological and histochemical analyses of tissue sections from the peritoneal cavity revealed the presence of tumor cell nodules and masses in adipose tissues by day 40 with progression to more extensive intraperitoneal spread. Tumor masses were identified along capsular and serosal surfaces of most major abdominal organs, with no parenchymal invasion. Tumor cells formed solid nests supported by a fine collagenous matrix as well as occasional small, irregular glandular structures. The omentum and mesentery were extensively colonized by tumor cells. The largest masses were present in the cranial abdomen (bordering the stomach, duodenum and pancreas). Dissemination of the peritoneal masses was attributed to transcoelomic spread given the general absence of organ involvement. There was no histologic evidence of tumor cells in distant organs such as heart or brain tissues.

Tissue sections were stained with biotinylated antibody 5F4 and WE scFv to verify Tn-antigen expression of ID8Cosmc-KO tumors. Consistent with the survival times of mice containing ID8 WT and ID8Cosmc-KO tumors, the extent of tumor spread, and size observed grossly and histologically was 10 to 20 days sooner for ID8Cosmc-KO.

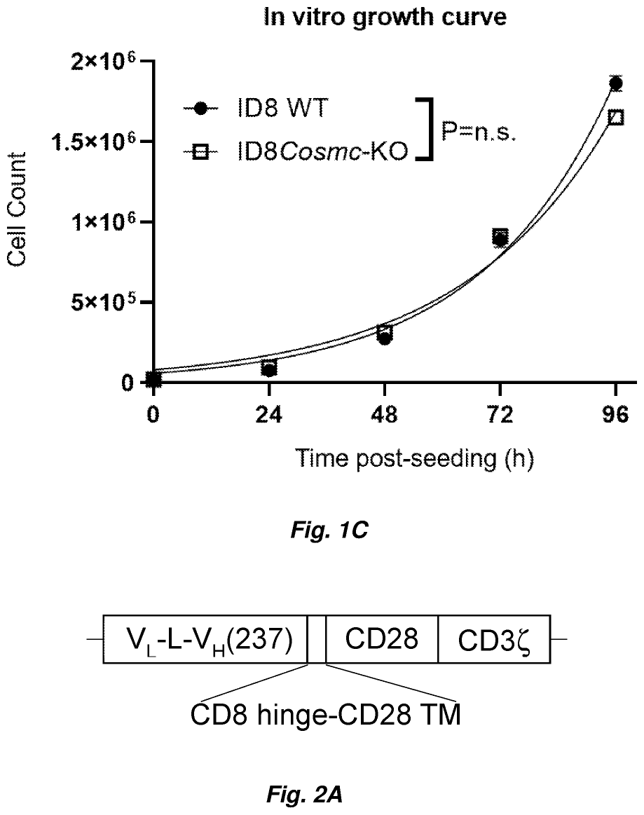

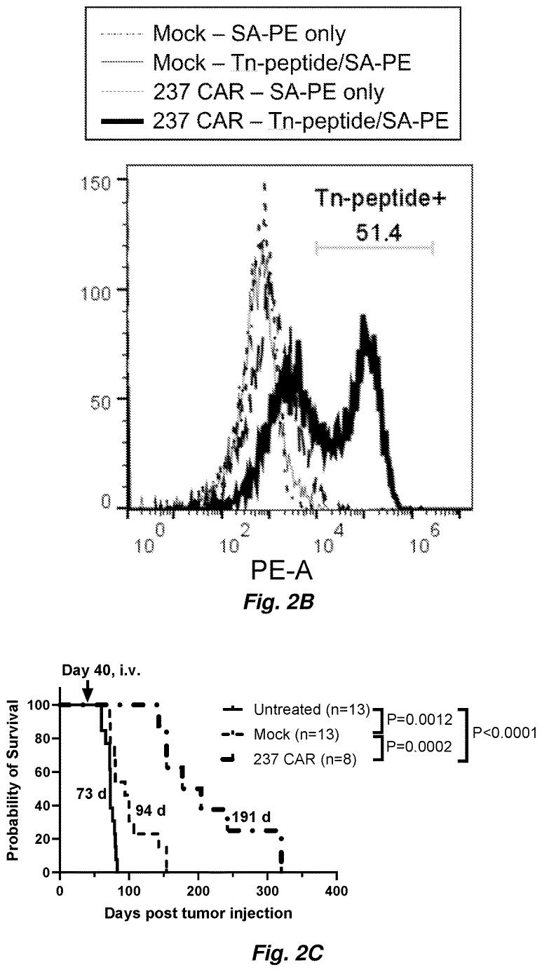

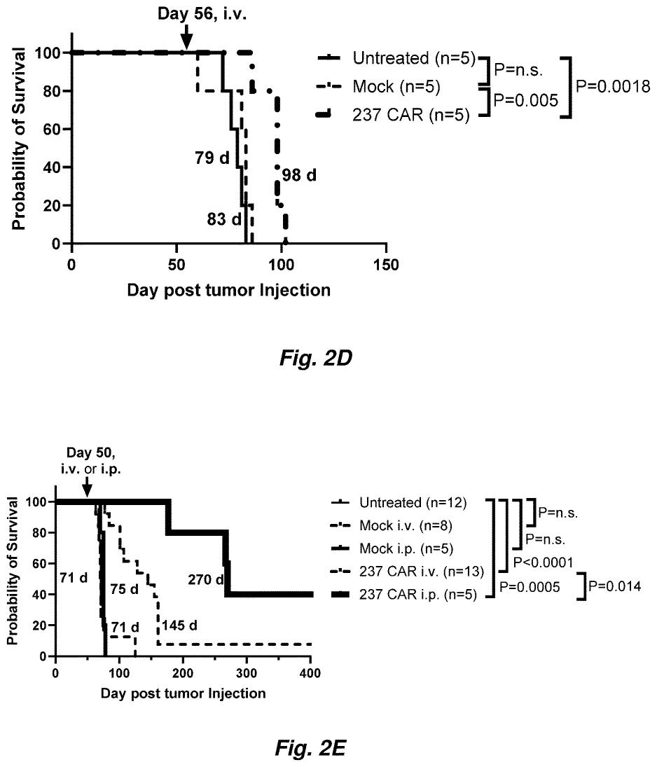

Efficacy of CAR-T cells in mice with established ID8 tumors: intravenous vs intraperitoneal treatments. We examined if 237 CAR-T cell treatment could delay the growth of ID8Cosmc-KO tumors, at times when solid tumors were well established. Activated splenic T-cells (total CD4 and CD8) from C57BL/6 mice were transduced with the pMP71 vector containing 237 CAR/CD28/CD32 (FIG. 2A). Transduced T-cells were analyzed by flow cytometry for CAR expression with biotinylated Tn-glycopeptide (FIG. 2B). Among multiple experiments, this analysis showed that 30% to 50% of transduced activated T-cells were positive.

Initial experiments involved intravenous administration of CAR-T cells or mock-transduced T-cells around day 40 after ID8Cosmc-KO transfer, one day after treatment with cyclophosphamide (FIG. 2C). Treatment with 237 CAR resulted in long-term control of tumors, with a median survival of 191 days (FIG. 2C). Mice treated with mock-transduced T-cells exhibited a delay in tumor growth to a median survival of 94 days, presumably due to the presence of activated polyclonal T-cells. Based on the exceptional result with CAR treatment on day 40, mice were treated on day 56 after tumor transfer (FIG. 2D). 237 CAR delayed growth compared to the untreated and mock controls (P=0.0018 and 0.0050, respectively).

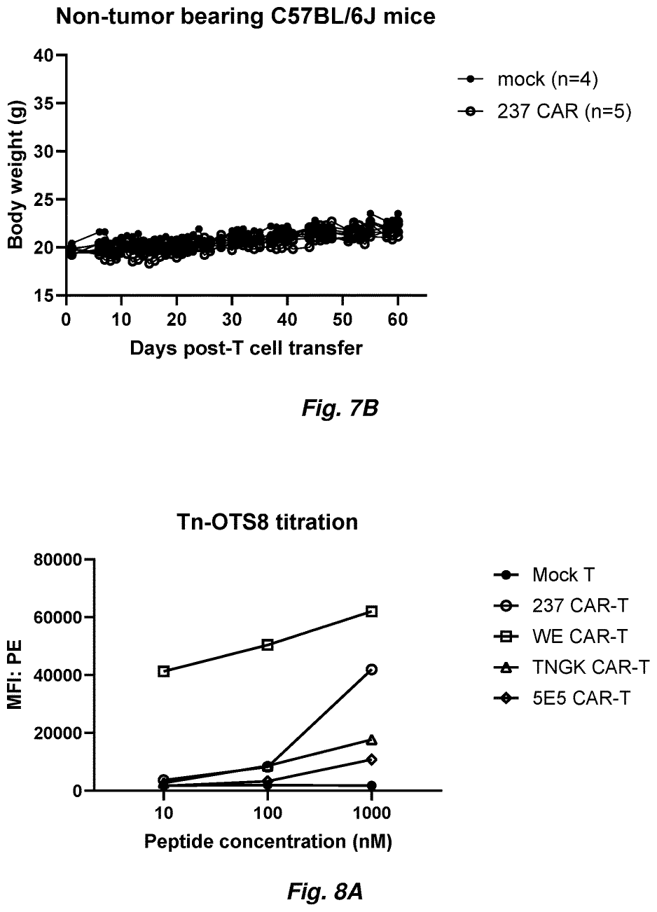

Ovarian cancer therapy can benefit from treatments that can be delivered directly to the peritoneal cavity, especially since tumors often develop on the surface of abdominal organs. To examine if intraperitoneal (i.p.) administration of CAR-T cells could control tumor growth better than i.v. delivery, we treated mice on day 50 after ID8Cosmc-KO transfer. Intravenous treatment with 237 CAR on day 50 slowed the growth of the tumor to a median survival of 145 days (FIG. 2E). Intraperitoneal delivery of the same CAR-T cells prolonged survival to 270 days (FIG. 2E). Importantly, no treatment-related adverse events were observed following i.p. injection of mock-T or 237 CAR-T cells in either tumor bearing or non-tumor bearing mice, based on gross pathological and histological examination of major organs (including peritoneal tissues) from CAR-treated mice (FIG. 7A-B).

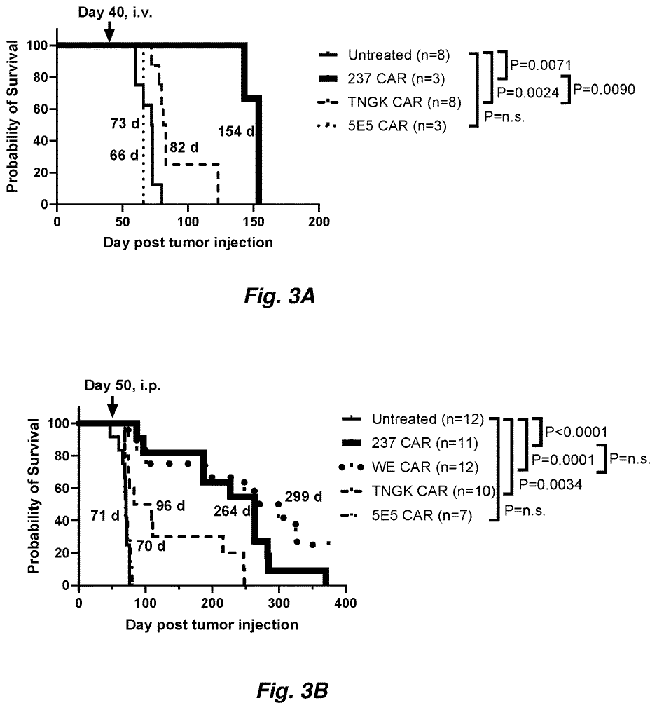

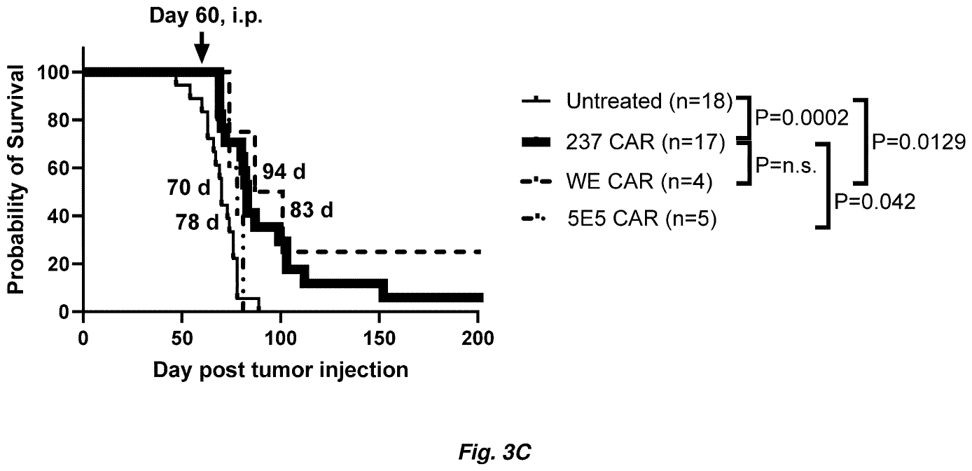

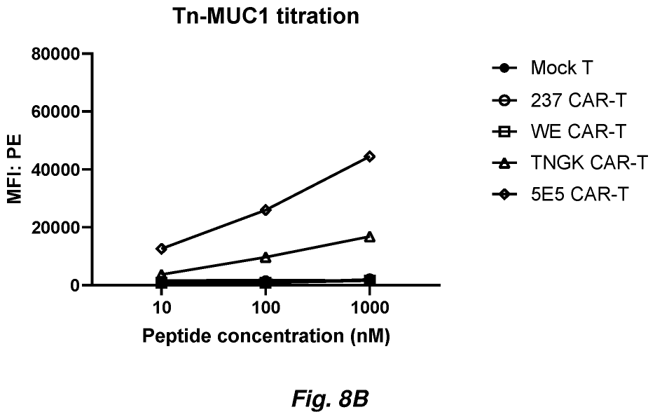

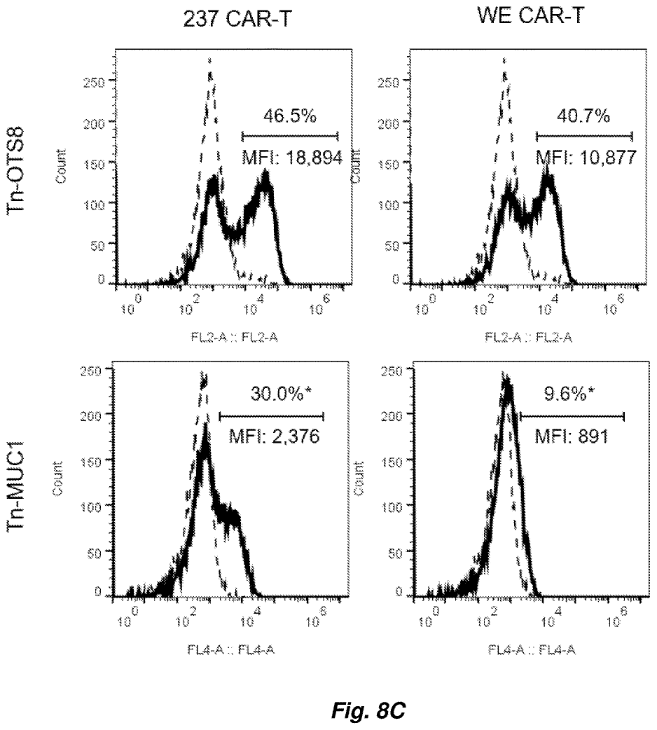

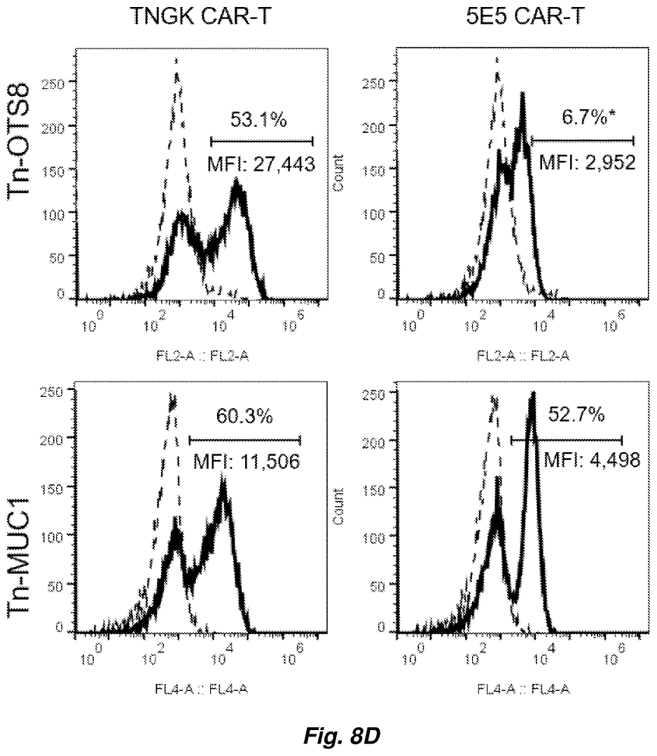

Efficacy of different Tn-dependent CARs in treatment of ID8Cosmc-KO tumors. We recently engineered a Tn-dependent CAR called TNGK, using the 237 scFv antibody backbone, that was selected with the human antigen Tn-MUC1. The TNGK scFv bound with higher affinity to Tn-MUC1 than 237 scFv, but it exhibited lower affinity binding to Tn-OTS8, the cognate mouse antigen recognized by 237 (FIG. 8A). Another Tn-dependent CAR called 5E5 was originally developed against Tn-MUC1 and has high affinity for Tn-MUC1 but low affinity for the mouse antigen Tn-OTS8 (FIG. 8A, 8B). TNGK and 5E5 CARs have about ten-fold less sensitivity than 237 CAR using immobilized Tn-OTS8 antigen to stimulate IFN-γ. Both of these CARs were tested in an experiment where mice with ID8Cosmc-KO were treated i.v. on day 40 (FIG. 3A). Although TNGK CAR delayed growth of the ID8 tumor, 5E5 showed no effect.

To extend these studies with additional Tn-dependent CARs, we also examined WE CAR-T-cells that contained the scFv variant with 30-fold higher affinity than 237 (K_{D} values of 120 nM and 4 nM for 237 and WE scFv, respectively). WE, 237, TNGK, and 5E5 CARs were administered i.p. at day 50 after ID8Cosmc-KO inoculation (FIG. 3B). 5E5 CAR showed no effect, TNGK CAR significantly extended median survival by 17 days (P=0.0034), and both 237 and WE CARs mediated long-term control of tumors, to median survivals of 264 and 299 days, respectively. There was no significant difference in tumor control between 237 and WE CARs, despite the 30-fold higher affinity of the latter.

To understand the limits of the CAR-T cell treatments, we performed an experiment in which mice were treated with 237 and WE CAR-T cells administered i.p. at day 60, the phase at which some mice reach the weight criterion for euthanasia. Not surprisingly, there was reduced efficacy of the CAR-T cell treatment at this advanced disease stage (FIG. 3C), but there was a significant delay in disease progression mediated by 237 and WE CARs (P=0.0009 and P=0.0095, respectively).

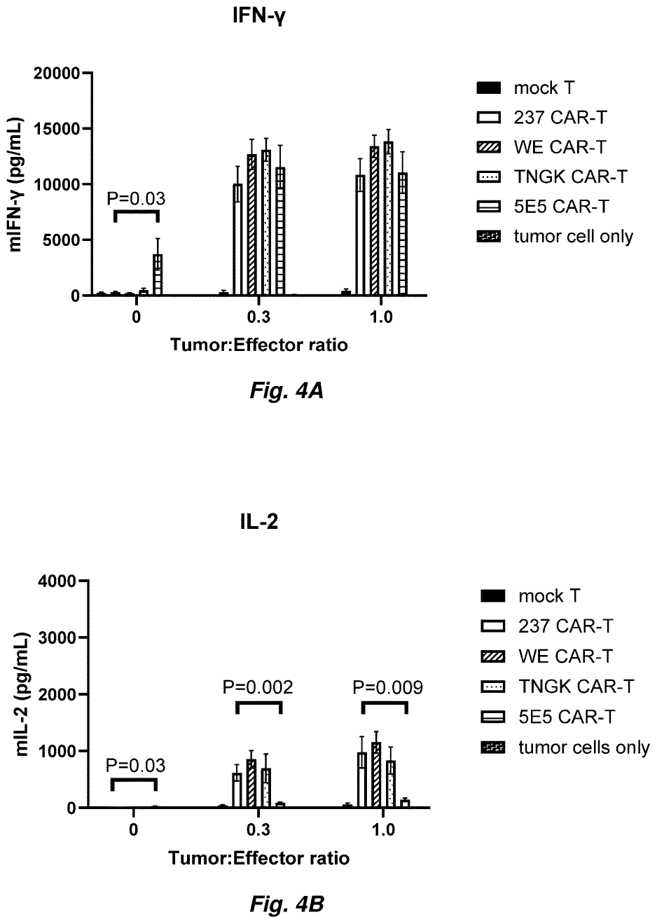

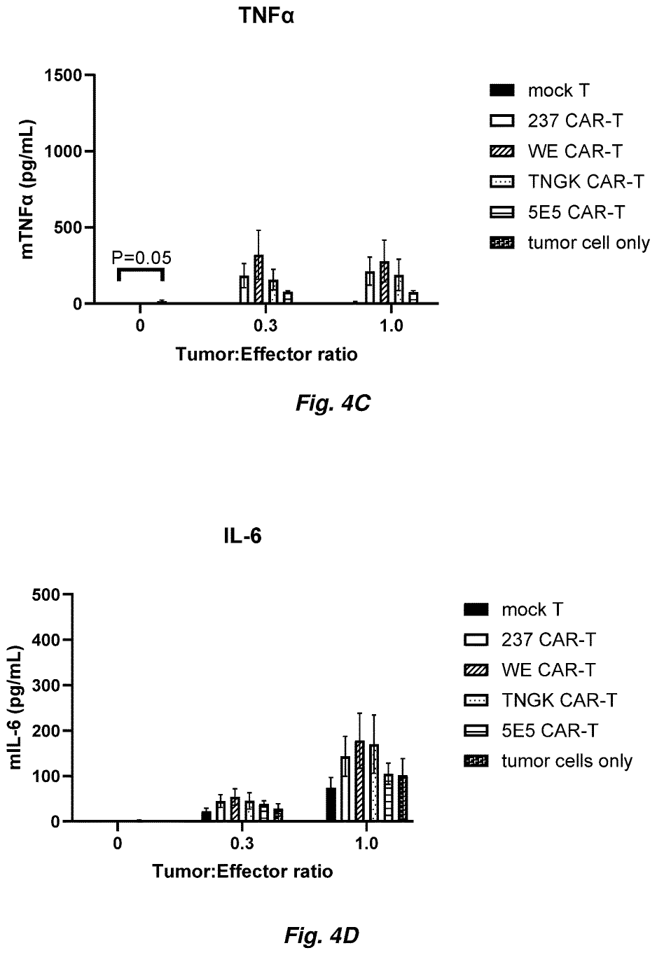

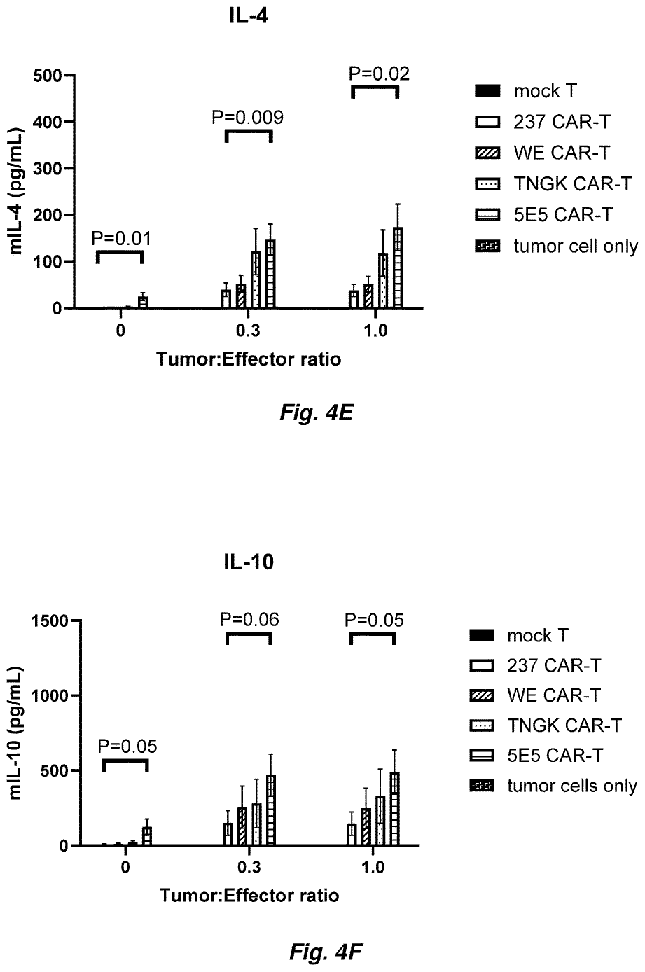

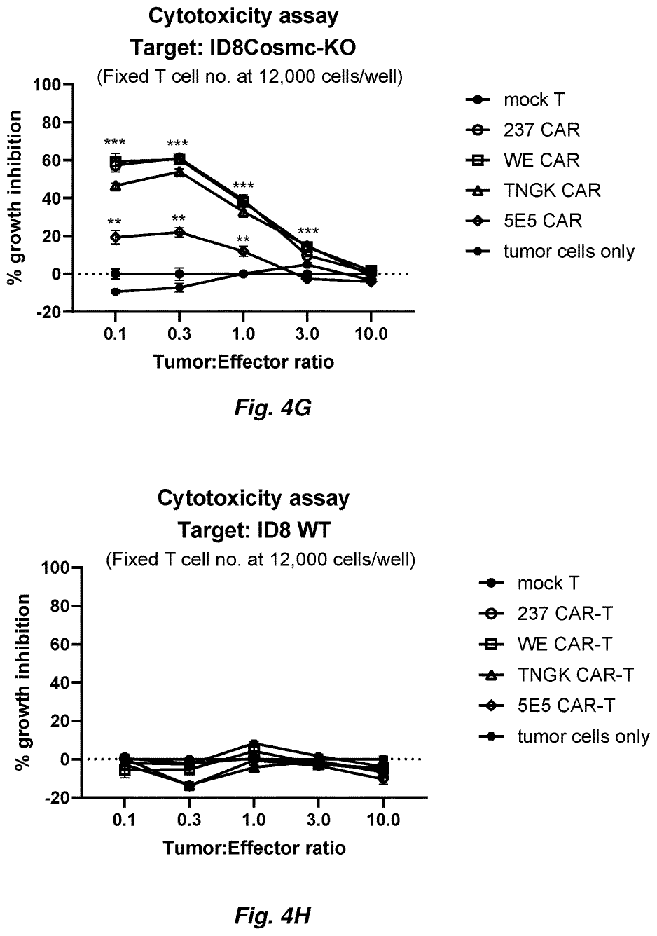

In vitro analyses comparing 237, WE, TNGK and 5E5 CARs. Given that all four CARs mediated Tn-dependent IFN-γ release in vitro when stimulated by ID8Cosmc-KO cells, the question remained what might explain their distinct differences in efficacy. To examine if there was a correlation between in vivo effects and in vitro cytokine release, we assayed in vitro-stimulated CAR-T cells for secretion of various cytokines, including IFN-γ, IL-2, TNFα, IL-6, IL-4 and IL-10 (FIG. 4A-F). To present antigen, we used various ratios of ID8Cosmc-KO tumor cells. Consistent with our previous findings, all four CARs were effective in mediating release of IFN-γ, even at the lowest tumor:CAR-T cell ratio of 0.1:1. Of particular note, among multiple experiments, the 5E5 CAR showed a level of tonic cytokine release, especially with IFN-γ, in the absence of antigen. While all four CARs mediated release of the other cytokines, there was a distinct quantitative difference among them. The two most effective CARs in vivo, 237 and WE, exhibited greater stimulation of IL-2 compared to 5E5. Interestingly, the reciprocal was observed for the release of IL-4 and IL-10, where 5E5 CAR exhibited higher levels than 237 and WE. However, 5E5 CAR-T cells also exhibited higher tonic levels of IL-4 and IL-10 than the other CARs, and significant levels of antigen-independent TNFα release. The release of IL-10 mediated by 5E5 CAR was also observed when it was co-cultured with Jurkat, a human tumor line with a known Cosmc mutation.

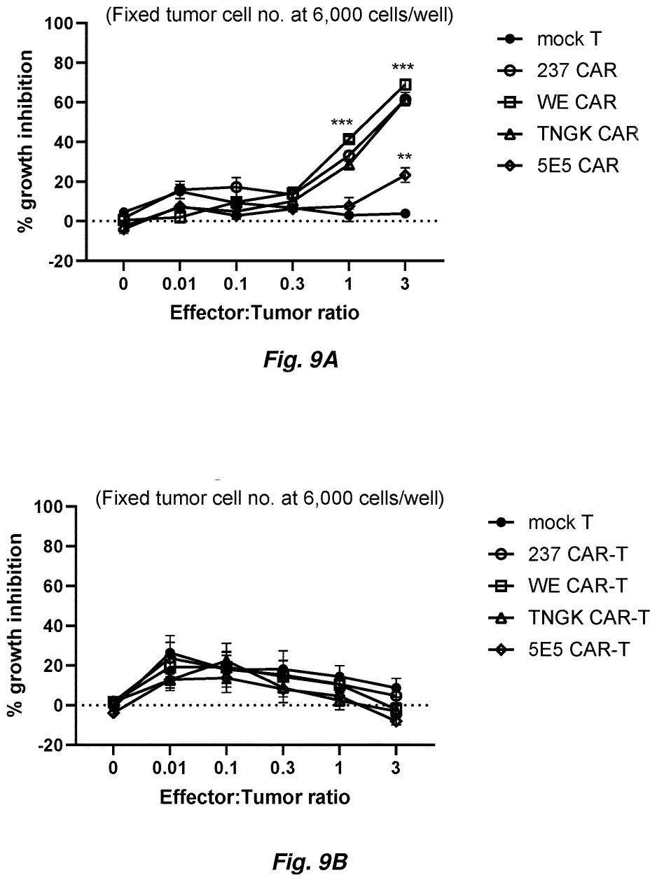

We also assessed in vitro tumor killing mediated by CAR-T cells. All four CARs mediated killing of ID8Cosmc-KO cells (FIG. 4E and FIG. 9A) but not ID8 WT cells (FIG. 4F and FIG. 9B). However, 5E5 CAR-T cells exhibited lower cytotoxic activity among the four CARs tested.

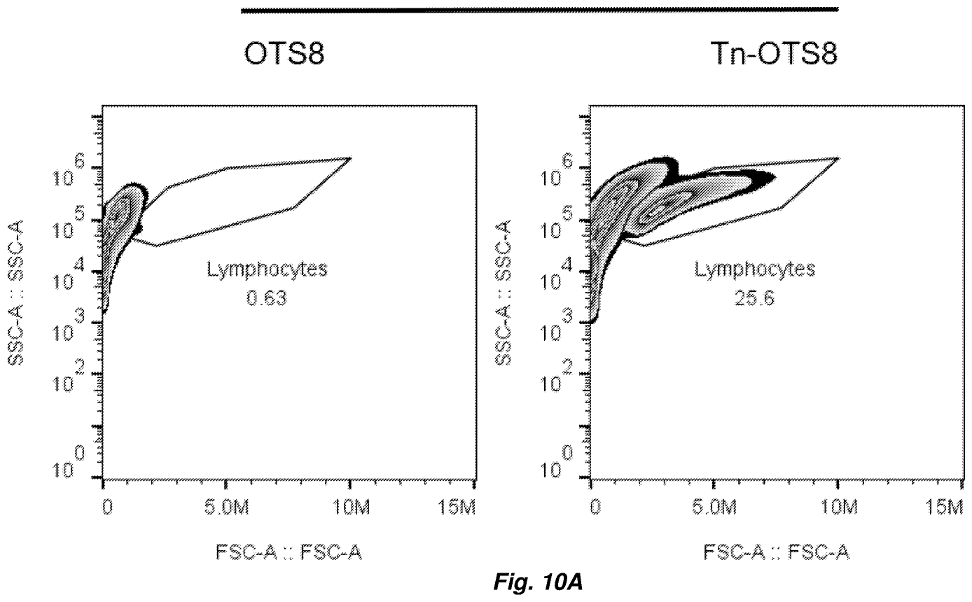

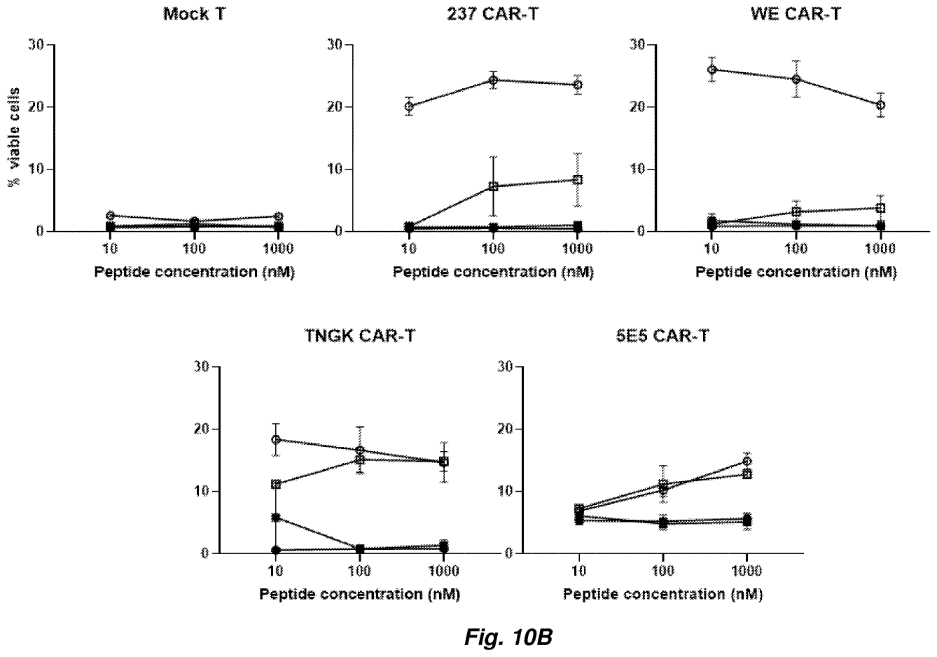

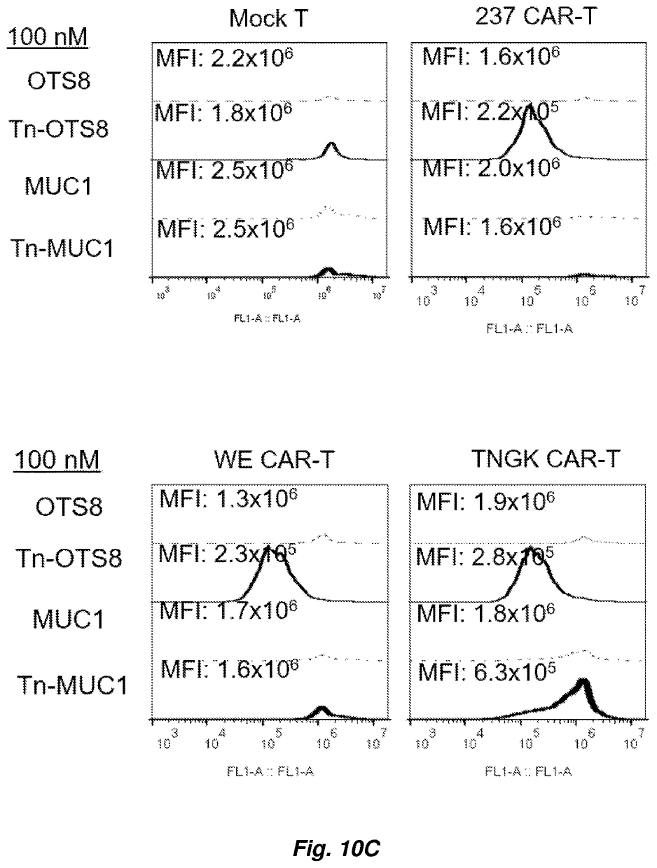

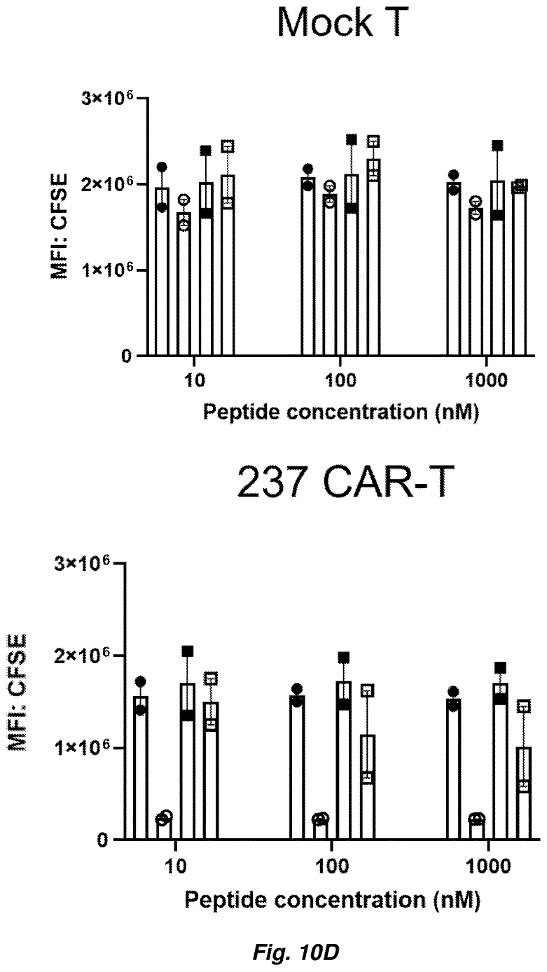

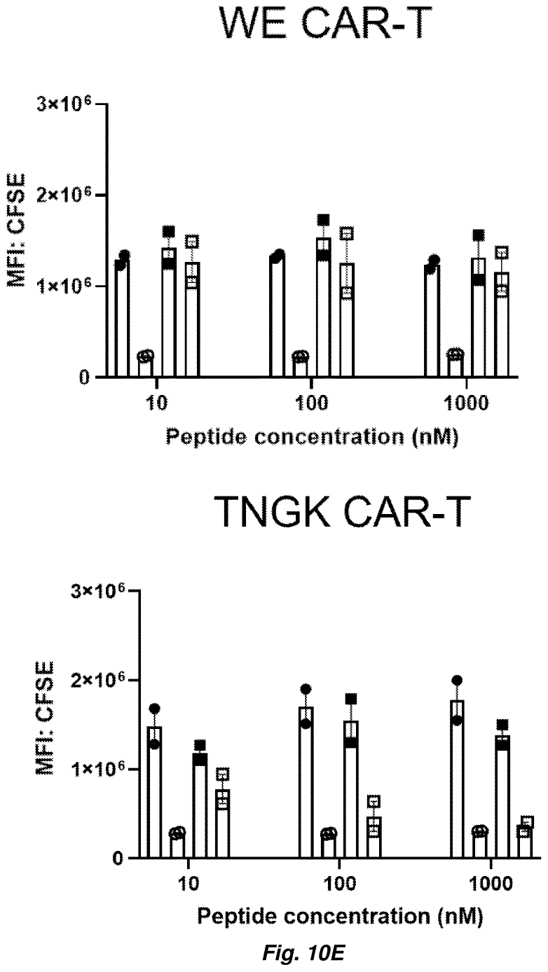

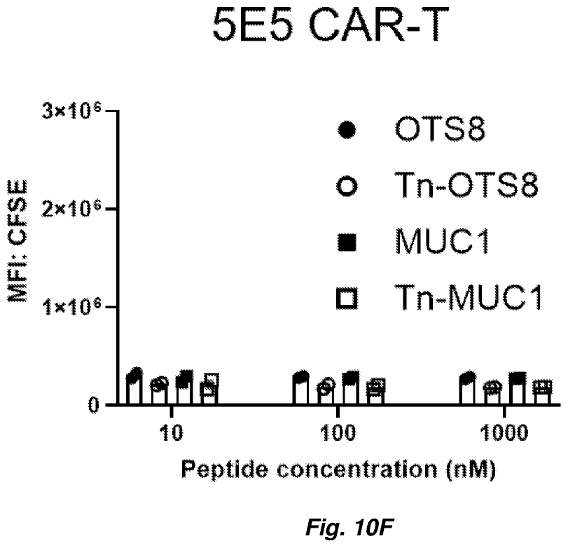

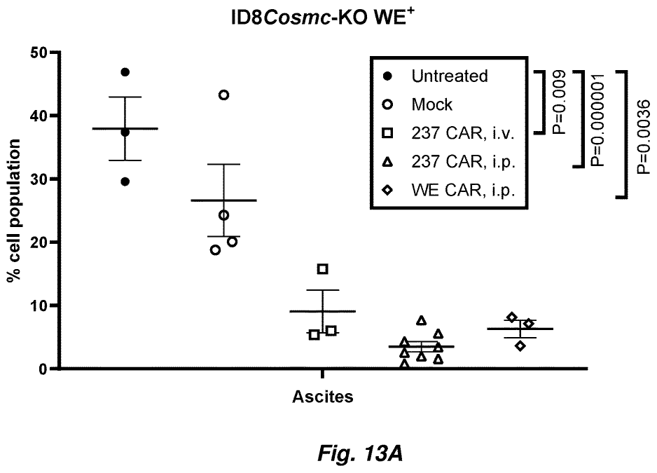

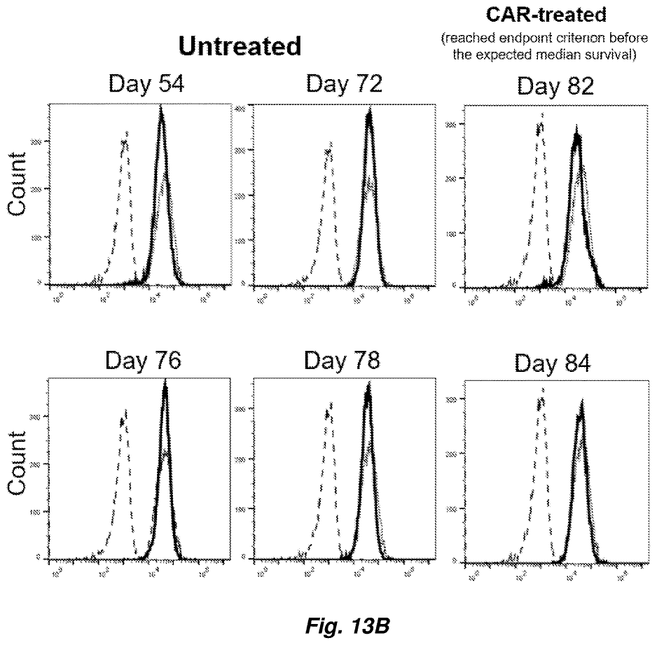

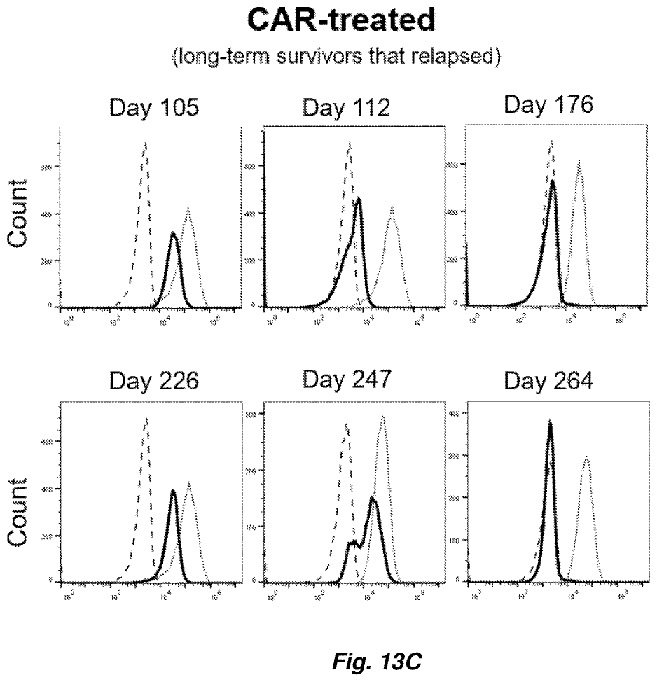

Finally, we examined antigen-dependent proliferation using CFSE-labeled T-cells stimulated with various concentrations of OTS8, Tn-OTS8, MUC-1, and Tn-MUC1 peptides for 72 hours by flow cytometry (FIG. 10). Gating on viable cells using forward/side scatter, we observed that 237, WE, and TNGK CAR exhibited background levels (mock T-cells) of proliferation in the absence of the cognate antigen, with about 0.5% in the lymphocyte gate (FIG. 10A, B). In contrast, 5E5 CAR exhibited almost 5% proliferating cells with the control peptides OTS8 and MUC1 (FIG. 10B). All the CARs, including 5E5, exhibited antigen-dependent proliferation, and for 237 and WE this level was highest with the mouse antigen Tn-OTS8. Similar results were observed when monitoring proliferation by flow cytometry for CFSE, where reduced MFI correlates with greater proliferation (FIG. 13C, D). The extent of proliferation was greatest for 237 and WE CARs with Tn-OTS8, whereas TNGK showed extensive proliferation with both Tn-OTS8 and Tn-MUC1. Again, 5E5 showed a high level of Tn-antigen-independent proliferation.

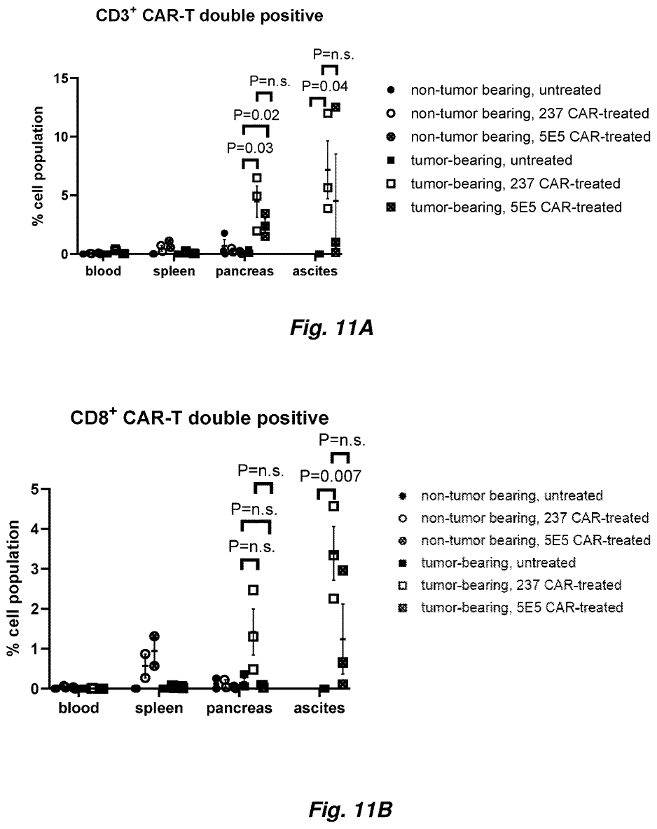

Immunophenotyping ascites and TILs from ID8Cosmc-KO mice. To assess CAR T cell distribution immediately after CAR treatment, we performed immunophenotyping by flow cytometry on samples from blood, spleen, pancreas, and ascites fluid recovered from ID8Cosmc-KO-bearing mice (FIG. 11). 237 or 5E5 CAR-T cells were administered i.p. on day 54 post-tumor challenge, and mice were euthanized three days post-T cell transfer. Although ascites at this time point is typically not large, the volume collected from 237 CAR-treated mice was higher relative to that collected from untreated mice. Ascites from 237 CAR-treated mice were ‘milky off-white’, compared to ‘red’ typical in untreated and 5E5 CAR-treated mice and the surface of pancreases from CAR-treated mice appeared more viscous compared to untreated controls.

We examined the distribution of CAR-T cells in circulation and within the pancreatic tissue using Tn-OTS8 tetramers. Both 237 and 5E5 CARs (CD3⁺, CD8⁺, and CD4⁺ double-positive cell populations) were detectable and significantly elevated in tumor tissues and ascites samples from CAR-treated tumor-bearing groups (FIG. 11), indicating that CAR-T cells, administered to mice three days prior, have migrated to target sites.

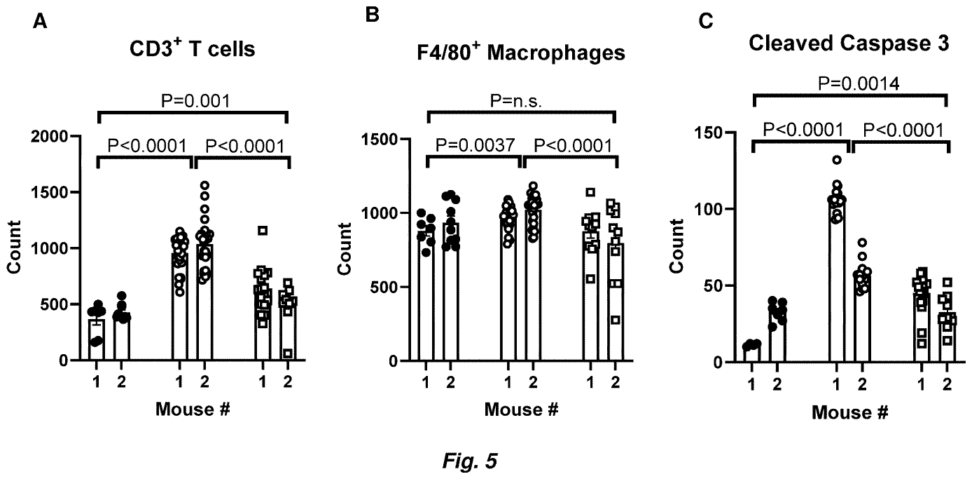

To further assess immune cell infiltration in tumors three days after CAR treatment, immunohistochemistry was performed with biotinylated WE-scFv, anti-CD3, anti-F4/80, anti-Ly6G, and anti-cleaved caspase 3 antibodies (FIG. 5). CAR-treated tumor sections contained prominent cellular infiltrates that outnumbered tumor cells. In the absence of CAR-treatment, few CD3⁺ T-cells were detected in tumors. Following either 237 or 5E5 CAR administration, tumor nodules and masses were extensively infiltrated by CD3⁺ T-cells, with 237 CAR-treated tissues showing significantly higher CD3⁺ T-cell infiltration compared to 5E5 CAR-treated tissues (FIG. 5, left panel). Based on flow analysis of TILs, it appears that only 5 to 10% of these CD3⁺ T-cells represent CAR-T cells (FIG. 11). The high frequency of infiltrating immune cells could partly explain the presence of sections that appeared negative for WE immunostaining, compared to tumors from untreated mice. While most of the small infiltrating cells had lymphocyte morphology and reacted with anti-CD3, there was also an increase in F4/80⁺ macrophage infiltration in all tumor sections examined, with 237 CAR-treated tissues exhibiting highest levels compared to either untreated or 5E5 CAR-treated tissues (FIG. 5, middle panel). Rare to small numbers of Ly6G⁺ cells (neutrophils) were observed in the tumor sections. Consistent with high levels of T-cell and macrophage infiltration, 237 CAR-treated tissues also had significantly higher number of apoptotic cells as shown in cleaved-Caspase 3 immunoreactive tissue sections (FIG. 5, right panel). We also examined the tumor stroma using Trichrome staining and found scant amounts of intratumoral fibrous tissue/collagen, arranged in thin, delicate strands, but not to an extent that we believe would interfere with CAR migration. Taken together, our results show that 237 CAR treatment recruited more CD3⁺ and F4/80⁺ macrophages to the tumor microenvironment, resulting in higher cleaved caspase 3-positive apoptotic cells compared to both 5E5 CAR treated and untreated tissues.

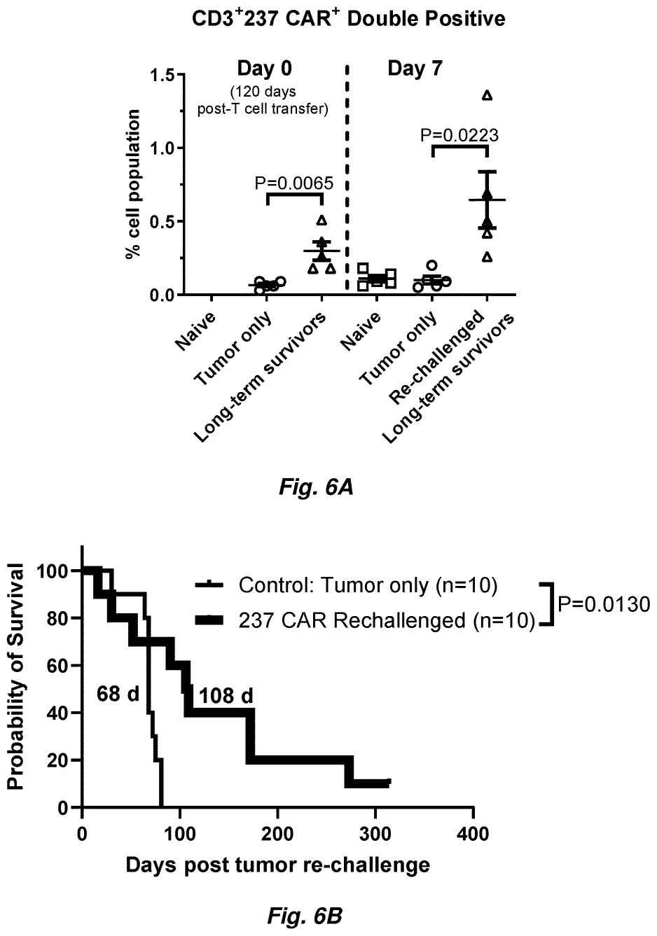

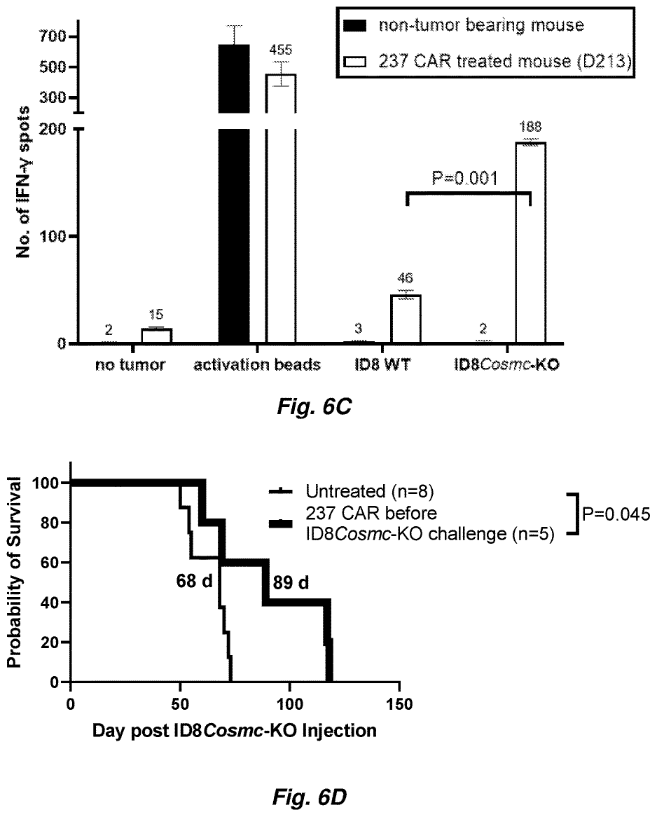

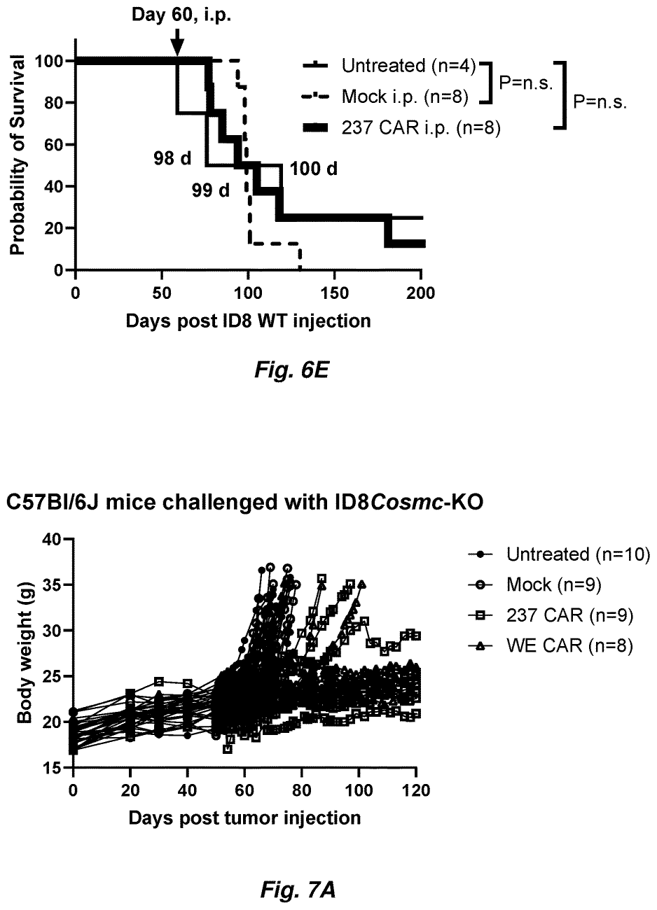

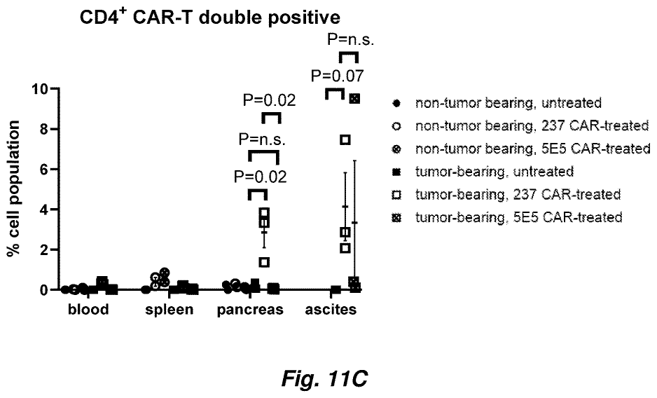

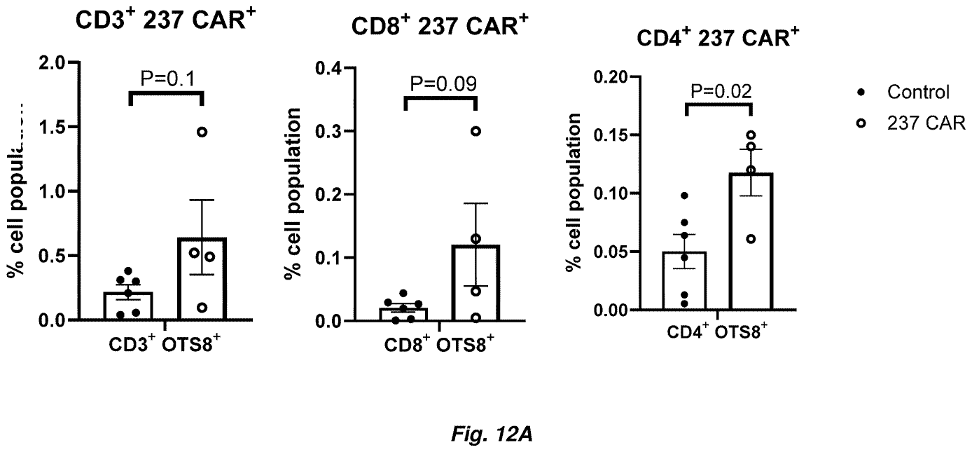

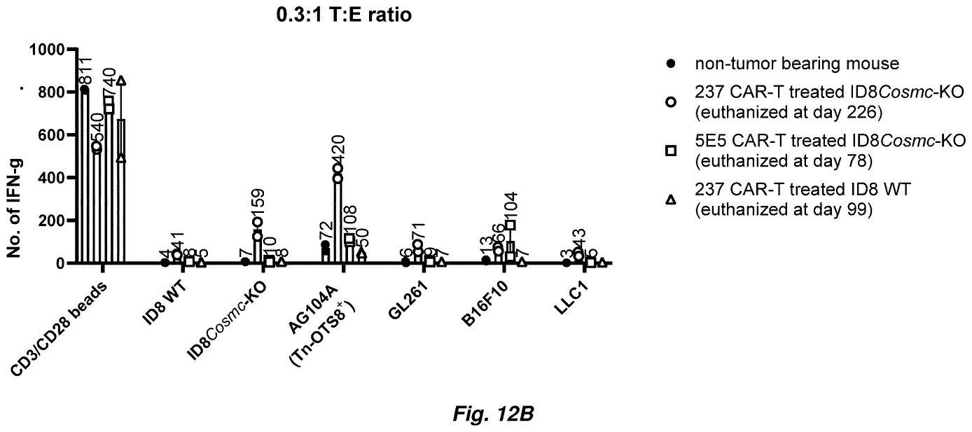

Persistence of CAR-T cells in treated mice. One of the metrics associated with efficacy of CAR-T cell treatments is persistence of CAR-T cells. To assess persistence, we tested the ability of CAR-T cell-treated mice to mount a response to re-challenge with ID8Cosmc-KO tumor, using mice that had controlled progression of tumor for a period of 120 days post T-cell administration. Prior to re-challenge, blood cells were co-stained with anti-CD3 and Tn-OTS8 tetramer, showing that CAR-T cells persisted, compared to age-matched non-tumor bearing mice (FIG. 6A, left panel). Seven days after re-challenge with ID8Cosmc-KO, the population of CD3⁺237 CAR T increased further (FIG. 6A, right panel). Survival data of re-challenged, CAR-treated mice (FIG. 6B) indicated that some mice were able to delay tumor progression, with a median survival of 108 days after re-challenge (compared to 68 days for naïve mice) (P=0.0130). In addition, analysis of blood 55 days post-re-challenge suggested that levels of CD4⁺CAR-T remained elevated in treated mice compared to control (FIG. 12A).

To further assess T-cell persistence, we performed IFN-γ Elispots of spleen cells from 237 CAR-treated mice. Splenic T-cells were co-cultured with ID8 WT or ID8Cosmc-KO tumor cells at various tumor-to-effector ratios. More cells from 237 CAR-treated mice were stimulated to secrete IFN-γ by ID8Cosmc-KO compared to ID8 WT cells (FIG. 6C), indicating that Tn-specific T-cells persisted in treated mice. Presence of Tn-dependent CAR T cells was also shown by the stimulation of IFN-γ secreting T-cells by the Cosmc-deletion line AG104A, but not other mouse tumor lines (FIG. 12B).

To explore whether antigen was necessary for CAR-T cell persistence, we examined whether CAR-T cells administered 60 days prior to tumor inoculation could delay tumor progression (FIG. 6D). CAR-T pre-treated mice survived to a median of 89 days compared to 68 days for untreated naïve mice, indicating that sufficient CAR-T cells were present, even in the absence of antigen, to delay growth (P=0.045).

Tn-antigen requirement in CAR-T cell treated mice. To verify that Tn-antigen was required for 237 CAR efficacy, we treated ID8 WT-bearing mice with 237 CAR (FIG. 6E). 237 CAR-treated mice did not show a delay in survival compared to untreated or mock treated controls, indicating that the in vivo potency of the 237 CAR requires Tn-antigens such as Tn-OTS8 generated by the ID8Cosmc-KO line.

Finally, to determine if long-term, relapsed mice treated with the 237 or WE CARs showed evidence of loss of the Tn-antigen, we analyzed the Tn-expression status of tumor cells collected from ascites of relapsed mice. The frequency of Tn-expressing tumor cells in ascites collected from 237- and WE-CAR-treated mice that reached endpoint was reduced compared to untreated and mock T treated groups (FIG. 13A). To directly address if the tumor cells in ascites from 237 CAR-treated mice contained lower level of Tn-antigen, we cultured cells from untreated mice (N=4), short-term treated mice (N=2), and long-term treated, relapsed mice (N=6). Tumor cell lines from untreated and short-term treated mice all expressed the same surface level of Tn-antigen (WE⁺) as the parental ID8Cosmc-KO line (FIG. 13B). In contrast, all six of the tumor lines derived from relapsed long-term survivors showed either lower levels of the antigen, or complete absence of detectable Tn-antigen. IHC of tissue sections from two long-term relapsed 237 CAR treated mice also demonstrated heterogeneity in Tn-expression in tumors.

### Discussion.

The mouse ovarian cancer line ID8 has been used in many previous studies to examine the efficacy of various therapies. However, successful treatment of this tumor has been difficult, requiring early treatments (i.e., often less than two weeks after tumor inoculation) to observe delayed tumor growth. In contrast, human ovarian cancer is rarely diagnosed at such early stages of disease development. Our most important finding is that late-stage treatments with CAR-T cells, when solid tumors are detectable by gross and histologic analysis, yielded long-term control of the tumor. Our studies also contrast with studies using human tumors transplanted into immunodeficient mice, which often do not express the human target antigen on normal mouse tissues which would be needed to assess toxicity.

Successful treatment was accomplished with just a single dose of 237 or WE CAR-T cells, even when ID8Cosmc-KO tumors were disseminated throughout the peritoneal cavity. Gross visual and histochemical analyses revealed tumor nodules and masses in adipose tissues by day 40 with progression to more extensive tumor spread by the time of CAR-T cell treatments on day 50 or 60. Several days after treatment, extensive T-cell infiltration into tumors was identified, yet there was no sign of CAR-T cell-related injury to neighboring organ systems. Although edema was observed in pancreas sections of CAR-treated mice, there was no evidence of toxicity in either tumor-bearing or non-tumor bearing mice in this syngeneic, immunocompetent system. The potency of the 237 CAR was associated with 237 CAR-driven immune cell infiltration which conferred an anti-tumor phenotype. This is consistent with a previous study showing that adoptively transferred T-cells are capable of overcoming immune suppressive cells. It was also interesting that the ID8Cosmc-KO tumor grew rapidly in Rag1-KO mice, indicating that endogenous B and/or T-cells are capable of controlling the tumor. Whether these cells are, or could be, recruited through the activity of 237 CAR-T cells remains to be examined.

Our results have relevance to the human disease, as CARs are in clinical development against several human ovarian cancer antigens, including folate receptor alpha (NCT03585764), mesothelin (NCT03916679, NCT03799913, NCT03814447, NCT05372692, NCT03054298, NCT05057715), B7-H3 (NCT05211557, NCT04670068), TAG72 (NCT05225363), MUC16 (NCT03907527), placental alkaline phosphatase, PLAP (NCT04627740), and P-MUC1 (NCT05239143). Pre-clinical studies against other targets, such as the Mullerian inhibiting substance type II receptor (MISIIR), have also shown promise. The optimal antigen target has yet to be determined, but development of multiple CARs against different targets provides opportunities to mitigate immune-evasive strategies employed by cancer cells such as antigen loss. In the present study, the potency of the Tn-dependent CARs ultimately resulted in reduced antigen expression in long-term survivors that eventually relapsed, but we have not yet elucidated the mechanism associated with the loss or reduction of Tn-antigen.

A trial using CAR T-cells expressing the humanized 5E5 scFv against Tn-MUC1 has been initiated (NCT04025216). In this regard, it is worth comparing the binding features of the 5E5 CAR and the 237, WE, and TNGK CARs. While all of these CARs are Tn-dependent, each has a different affinity and/or peptide fine-specificity. Surface plasmon resonance (SPR) has been used to determine the affinity of the parental antibodies and their antigens: 5E5 has an affinity (K_{D}) of 2 nM for human Tn-MUC1 and 237 has an affinity (K_{D}) of 140 nM for mouse Tn-OTS8. The WE scFv was derived from 237 using in vitro affinity maturation to yield an affinity increase of 30-fold, to an affinity of 4 nM for Tn-OTS8 (i.e., similar to 5E5 for Tn-MUC1). The TNGK scFv was engineered to bind to Tn-MUC1. WE and TNGK CAR differ in their CDRs from 237 CAR by only two and four amino acids, respectively.

While affinities using SPR have not been determined for these scFv:Tn-glycopeptide pairs, based on antigen titrations the order of binding to the predominant mouse antigen in ID8Cosmc-KO, Tn-OTS8, is WE>237>TNGK>5E5 (FIG. 10A). The order of binding to human Tn-MUC1 is 5E5>TNGK>237>WE. Although all these CARs exhibit a degree of cross-reactivity with different Tn-peptide backbones, we show in this report that affinities of 140 nM (237 for Tn-OTS8) or higher may be equally optimal for in vivo efficacy, at least in the context of antigen densities represented with ID8Cosmc-KO. The reduced in vivo efficacy of the TNGK CAR may be accounted for by its lower affinity than 237 and WE for the Tn-OTS8 antigen on ID8Cosmc-KO. An affinity threshold for the scFv fragments used in CARs has been previously described (Chmielewski et al., J Immunol. 2004; 173 (12): 7647-53).

Another observation of interest is that despite the ability of all four CARs to mediate in vitro release of IFN-γ, stimulation of other cytokines differed among the CARs. It was particularly notable that the most efficacious CARs in vivo showed higher IL-2 but lower IL-10 and IL-4. In contrast, 5E5 CAR-T cells secreted higher relative levels of IL-10 and IL4. This observation is consistent with the well-known immunosuppressive abilities of IL-10.

Perhaps most decisive with regard to the mechanism underlying the lack of efficacy of the 5E5 CAR was our observation that it yielded a low level of tonic activity, unlike the other three CARs. A head-to-head comparison of 237-CAR and 5E5-CAR (both with CD28 signaling domain) showed that the 5E5 CAR used in this study exhibited basal tonic activity and proliferation, and about a two-fold lower level of surface expression compared to other CARs (FIG. 10B). Tonic, antigen-independent activity of CARs has been reported to be due to aggregation of scFv. The V_{H} and V_{L} domains of 5E5 are quite different from the V_{H} and V_{L} domains of the 237, WE, and TNGK CARs, which have identical framework regions. Importantly, tonic activity has been correlated with ineffective CARs, and one way to mitigate this tonic stimulation is through CAR engineering. It is worth noting that two previous studies used 4-1BB instead of CD28 in their 5E5 CAR constructs, which could explain why these 5E5 CAR constructs did not yield tonic activity. The use of the TNGK-CD28 CAR for human translational studies, on the other hand, may be advantageous given its favorable properties of low tonic release and its recognition of human Tn-MUC1.

The question remains what surface level of Tn-protein antigen in human cancers is minimally sufficient to elicit CAR T-cell efficacy, as described here. The 5E5 CAR has been shown to mediate killing of, and IFN-γ stimulation by, a panel of diverse human cell lines (6 out of 10 cell lines). This activity was correlated with upregulation of the ST6GalNAc1 transferase, or in one case (Jurkat) the mutation of Cosmc. Among the limited tumor cell lines that we have examined with 237 or its derivatives (including WE and TNGK) using transduced mouse T-cells, a Cosmc deletion was necessary for Tn-dependent activity. The high density of Tn-targets found in Cosmc deletion mutants may correlate with the optimal in vitro cytokine stimulation and proliferative activity, and in vivo tumor infiltration of the 237 and WE CARs, as described here. Nevertheless, the frequency of human tumors that exhibit Cosmc mutations may make treatment with Tn-dependent CARs a valuable option for some patients. Endometrial cancers are reported to have a 5% prevalence of Cosmc-mutations, and other cancer types including ovarian cancer are in the range of 0.5 to 1%. While this may seem low, current efforts to target a specific driver mutation (e.g., in p53 or KRAS), combined with the requirement for HLA-restriction bring these frequencies to the same range.

Our findings that intraperitoneal administration of the CAR-T cells was more effective than intravenous administration supports such an approach in clinical trials of CAR-T cells against ovarian cancer. Furthermore, the potency of a single CAR T-cell treatment at days 40 to 50 after tumor transplant indicates that post-surgical treatment, when there might be minimal but significant disease, could be a preferred time for optimal therapeutic benefit. This is also particularly relevant to a recent study in which ID8 was implanted into the ovaries; despite surgical debulking at different times, all mice exhibited ID8 tumor development with pathology and dissemination much like the ID8Cosmc-KO model described here.

A notable exception for later treatment of the ID8 model involved the use of an “armored” CAR that expressed a synthetic IL12 gene. It remains to be seen if CAR-T cells engineered to secrete IL12 or other cytokines have appropriate safety profiles, as IL12 has shown serious toxicities clinically. Inducible systems have been designed to mitigate potential cytokine toxicity associated with these types of CARs. While the induction of some cytokines in the latter system delayed tumor growth, the exact cytokine balance that will drive optimal CAR-T cell efficacy in different solid tumors is unknown.

As with CAR or TCR-based cellular approaches and other immunotherapies, combination therapies that target other cancer antigens or pathways would be expected to reduce the chances of loss of any single tumor antigen. Combination with oncolytic viruses for induction of innate and NK cell responses and vaccines that elicit endogenous T-cell activity could be key combination therapies to sustain tumor control, without the emergence of CAR-antigen loss variants. There is some evidence that endogenous T-cells can be recruited in the ID8 tumor model, but their existence in the ID8 CAR system described here remains to be seen. Nevertheless, our findings using a single dose of CAR T-cells are even more notable given that other strategies would likely provide greater benefit through synthetic engineering, recipient T-cell selections, or combination treatments.

### Example 2. Materials and Methods

Cell lines. Murine ovarian cancer cell line, ID8, and its mutant line with Cosmc gene deletion (ID8Cosmc-KO) were maintained in complete Dulbecco's Modified Eagle Medium (DMEM) supplemented with insulin-transferrin-sodium-selenite supplement at 1:5000 dilution. The generation of ID8Cosmc-KO using CRISPR/Cas9 has been described (He et al., JCI insight. 2019; 4 (21); Sharma et al., Proc Natl Acad Sci USA. 2020; 117(26):15148-59). Cell lines were submitted to IDEXX BioAnalytics for authentication, and were tested free of pathogens. All cell lines and primary cells used in this study were cultured at 37° C. with 5% CO₂.

Chimeric antigen receptors. The 237 scFv was fused to CD8α hinge-CD28-CD3ζ and cloned into the pMP71 retroviral vector to generate 237 CAR plasmid (Stone et al., OncoImmunology. 2012; 1(6):863-73). Structure-guided engineering of the 237 scFv fragment (Sharma et al., Proc Natl Acad Sci USA. 2020; 117(26):15148-59) yielded CAR variants that bound with higher affinity to the cognate murine Tn-OTS8 peptide (237 scFv mutant WE, designated WE CAR here) or exhibited binding to both murine Tn-OTS8 and human Tn-MUC1 (237 scFv mutant TNGK, designated TNGK CAR here). The 5E5 scFv CAR that binds to human Tn-MUC1 was characterized in human T-cells previously (He et al., JCI insight. 2019; 4(21)) and was subcloned into the pMP71 for mouse T-cell studies.

T-cell transduction and activation. The protocol for retroviral transduction of primary T-cells isolated from splenocytes of donor mice are known in the art and described, for example, in Stone et al., OncoImmunology. 2012; 1(6):863-73; Sharma et al., Proc Natl Acad Sci USA. 2020; 117(26):15148-59. Mock-transduced T-cells went through the retroviral transduction process but without recombinant virus. Transduction efficiency was measured using Tn-OTS8 tetramer for 237, WE and TNGK CARs or Tn-MUC-1 tetramer for 5E5 CARs.

Animal studies. Animal studies were approved by the UIUC Institutional Animal Care and Use Committee (IACUC) under protocol numbers 21041 and 20176. Female C57BL/6J mice (8-10 weeks old) were purchased from Jackson Laboratories, maintained under pathogen-free conditions in a barrier facility, ear-tagged, and assigned to treatment groups. ID8 and ID8Cosmc-KO tumor cells (10⁷) were inoculated intraperitoneally. After various days, 5×10⁶ CAR-T cells (or mock-transduced T-cells) were administered intravenously or intraperitoneally. At 24 hours prior to T-cell transfer, cyclophosphamide (100 mg/kg) was administered i.p. Tumor growth and progression of disease were monitored by measuring body weight on a regular basis. Mice were euthanized when their body weights reached endpoint criterion of 36 g or a gain of 50% of their weight from the start of treatment. For some experiments, blood samples (~70 μL) were collected by submandibular vein puncture or retro-orbital bleeding. Tissue samples were collected postmortem, fixed in 10% buffered formalin for 24 hours, and subsequently transferred to 70% ethanol prior to routine tissue sectioning at 5-micron thickness and staining with hematoxylin and eosin (H&E).

Antibody detection agents. Antibodies used for flow cytometry were purchased from BioLegend or BD Bioscience: CD3 (clone 17A2), CD8α (53-6.7), CD4 (RM4-5), CD45 (30-F11), and TCR Cβ (H57-597). Fixable Viability Dye eFluor 780 was purchased from eBioscience (Invitrogen 50-169-66). Data were acquired on a BD LSRII and analyzed with FlowJo software. The Tn-specific monoclonal antibody 5F4 (IgM), a kind gift from Dr. Henrik Clausen (University of Copenhagen, Denmark), was used as culture supernatant (for flow cytometry) or purified using LigaTrap Microspin Kit (LT-145KIT) and biotinylated (ThermoFisher/Pierce EZ-Link NHS-PEG4 biotinylation kit 21455) (for immunohistology). The 237 scFv mutant WE, cloned into pET28a with a 6× histidine and Avitag, was purified from inclusion bodies and biotinylated using BirA ligase from Biotin-Protein Ligase Reaction Kits (Avidity Inc. BIRA-500). Fluorescently labeled WE scFv tetramers were prepared by combining biotinylated WE scFv with streptavidin-PE (BD Bioscience CN554061) or streptavidin-Alexa Fluor 647 (Invitrogen CNS32357) at a 4:1 molar ratio. Biotinylated Tn-OTS8 and biotinylated Tn-MUC1 peptides were prepared as described.

Processing and analysis of tissues and tumors. Protocols for processing mouse tissues and staining for flow cytometry are described in the art (see Ranoa et al., J Immunother Cancer. 2023 May; 11(5)). H&E staining and immunohistochemistry of tissue sections from formalin-fixed paraffin embedded (FFPE) samples were performed by the University of Chicago Human Tissue Resource Center core facility. Stained slides were scanned using a Hamamatsu NanoZoomer slide scanner. Protocols for staining with biotinylated agents (anti-Tn antibody 5F4 or WE scFv), anti-CD3 (Abcam ab135372, clone SP162), anti-F4/80 (BioRad MCA497GA, clone A3-1), anti-Ly6G (BioLegend 127602, clone 1A8), and anti-cleaved caspase 3 are known in the art.

In vitro co-culture assays. ID8 WT or ID8Cosmc-KO cells were co-cultured with CAR-T-cells in 96-well plates at different seeding density. In one approach, the number of effector T-cells was kept constant (12,000 cells/well) while the number of tumor cells was varied (tumor:effector ratios). In another, tumor cell numbers were constant while effector T-cell numbers were varied. After co-culturing for 24 hours at 37° C., supernatants were transferred to V-bottom 96-well plates and supernatants were stored at −20° C.

For cytokine analysis, 25 μL supernatants were used in customized mouse cytokine array (Milliplex MCYTOMAG-70K) following manufacturer's protocol. The panels included IFN-γ, IL-2, IL-4, IL-6, IL-10, and TNFα (Milliplex MCYTOMAG-70K-6plex). Plates were read on Luminex 200, with quality controls and standards included.

T-cell cytotoxicity of tumor cells was measured using the Sulforhodamine B colorimetric assay as described in Vichai et al., Nat Protoc. 2006; 1 (3): 1112-6. IFN-γ ELISPOT assays were performed using splenic T-cells and a mouse IFN-γ ELISPOT kit (CTL ImmunoSpot), following 24-hour co-culture.

T-cell proliferation assays were performed by labeling activated mock or CAR-T cells with CFSE (Invitrogen C34554) following the manufacturer's protocol. Post-transduction (72 hrs), T-cells were washed, resuspended in PBS, and incubated with 10 μM CFSE for 20 minutes at 37° C. in the dark. Cells were washed with culture media, and resuspended to a final cell density of 1×10⁶ cells/mL. 100 μL cell suspension was aliquoted in 96-well plate pre-coated with streptavidin followed by biotinylated peptides. Following 72-hour incubation, T-cells were harvested, washed, and analyzed in a BD Accuri flow cytometer.

### Example 3. Combination PAC-1 and CAR T-Cell Treatment of Cancer

### Efficacy of PAC-1 and 237 CAR-T Combination.

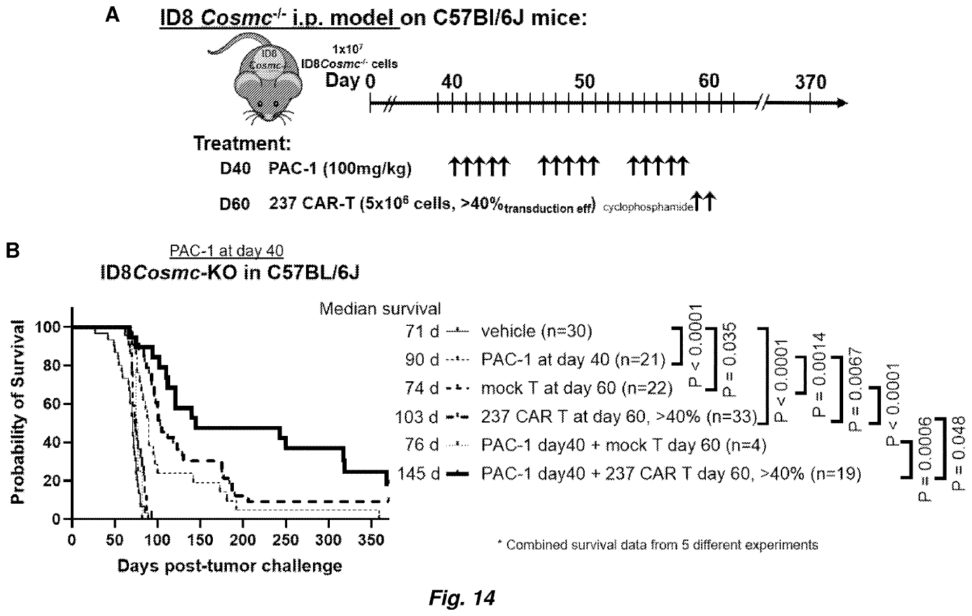

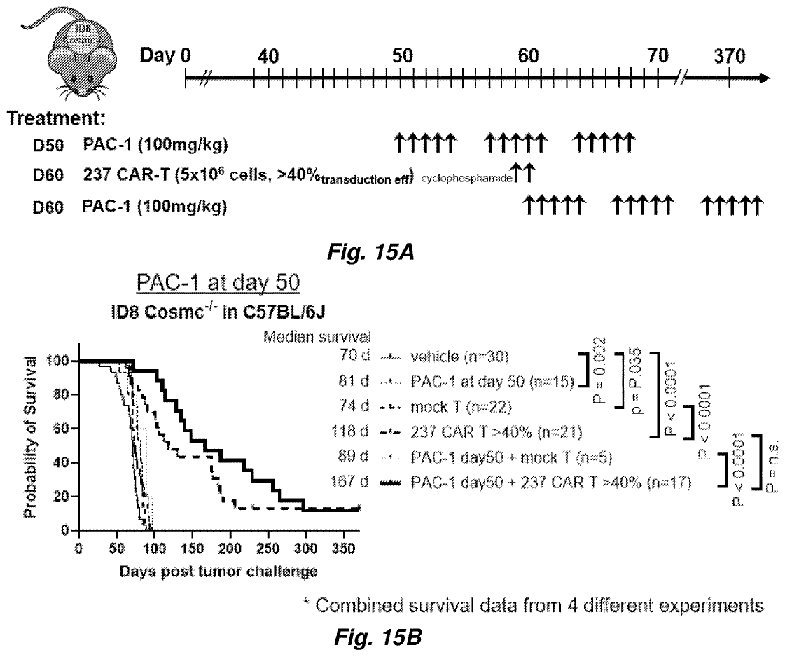

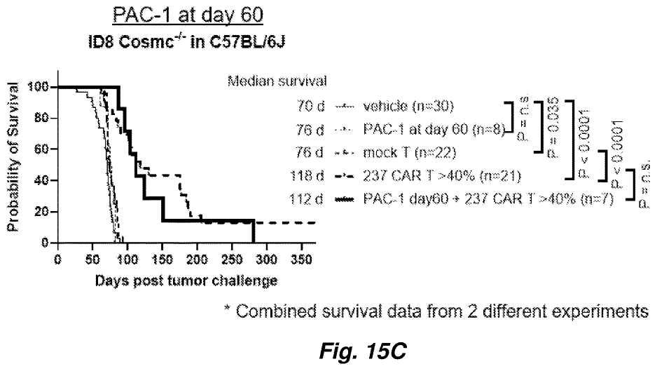

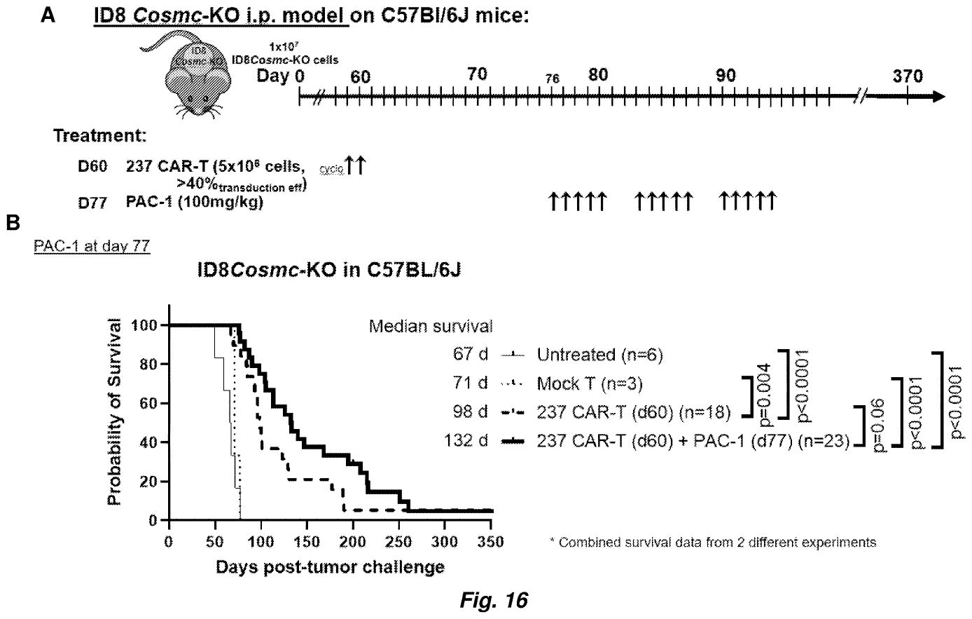

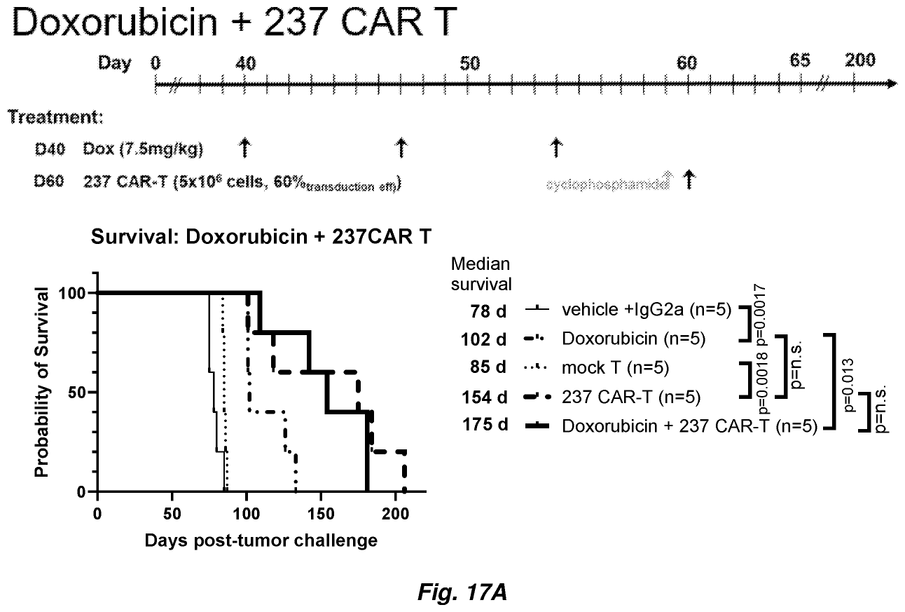

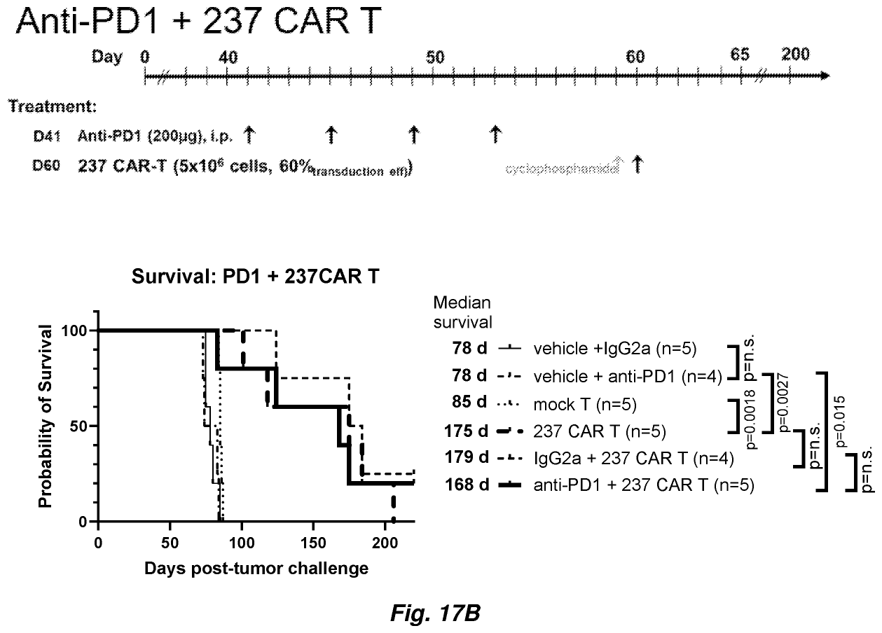

To test if small molecule PAC-1 can be used in combination with 237 CAR-T, we performed in vivo experiments using our murine ID8Cosmc-KO ovarian tumor model, in which tumor-bearing mice were treated with 237 CAR-T cells administered i.p. at day 60 and were also given PAC-1 i.p. at different time points before or after CAR-T cell injection. In our model, untreated mice with disseminated tumors have a median survival of 71 days (FIG. 14A-B). As a single agent administered intraperitoneally, PAC-1 extended the median survival to 90 days (p<0.0001), while 237 CAR-T treated mice have a median survival of 103 days (p<0.0001). A combination of the two treatment modalities (PAC-1 administered before 237 CAR-T) further extended the median survival to 145 days (p=0.048) (FIG. 14A-B). However, when PAC-1 was administered at a later time point, either at day 50 (FIG. 15A-B) or day 60 (FIG. 15C), no significant difference was observed between 237 CAR-T alone or in combination with PAC-1, suggesting that the mode of action of PAC-1 involves activating the host adaptive immune response in addition to its known apoptotic activity. Administering PAC-1 after 237 CAR-T also modestly extended the median survival to 132 days (p=0.06) (FIG. 16). We further tested the combination of 237 CAR with either Doxorubicin (FIG. 17A) or the checkpoint inhibitor anti-PD1 antibody (FIG. 17B), but in both approaches, no significant difference was observed between 237 CAR-T alone and in combination with either Doxorubicin or anti-PD1 antibody. Similarly, we determined if we can further enhance the efficacy of PAC-1 and 237 CAR-T combination by adding anti-PD1 antibody as a third modality (FIG. 17C), but this triple combination approach did not lead to better outcomes compared to 237 CAR-T as a single agent. Taken altogether, our results demonstrate that PAC-1 engages the immune system to treat cancer by inducing expression of pro-inflammatory cytokines in tumor cells allowing the recruitment of CD8+ effector T cells to the tumor microenvironment. Combination strategies that boost T cell trafficking shows great promise to enhance the anti-tumor efficacy and feasibility of adoptive CAR-T therapy against solid tumors.

### Persistence of CAR-T Cells in Treated Mice.

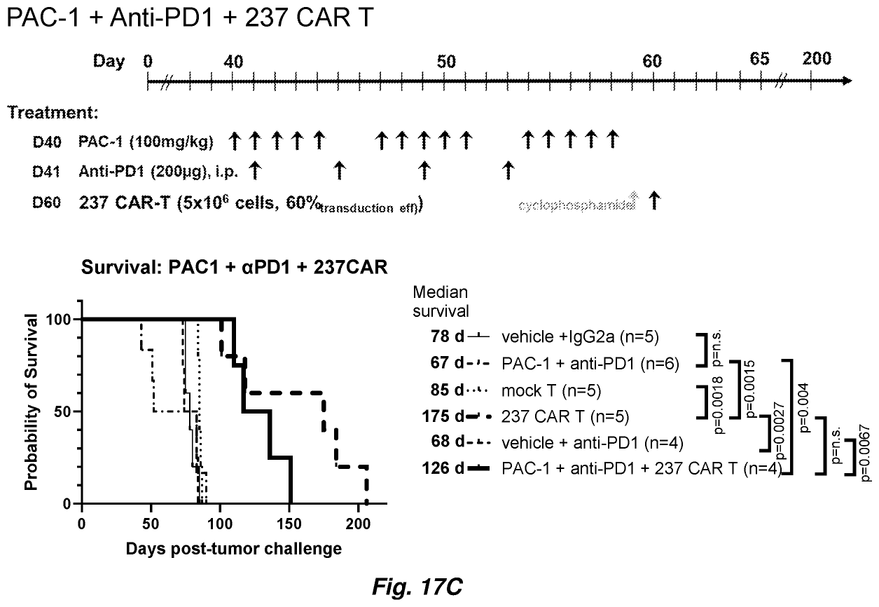

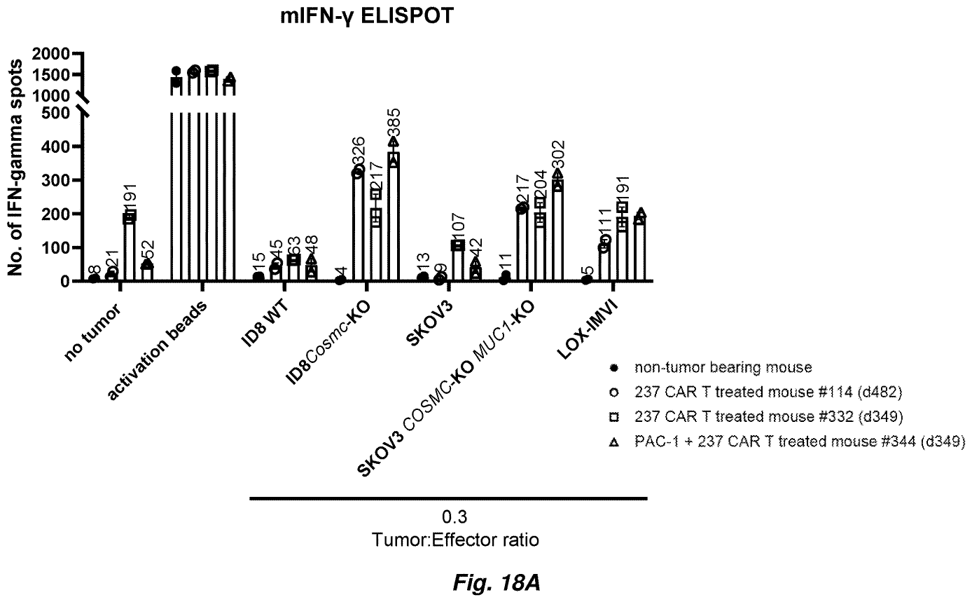

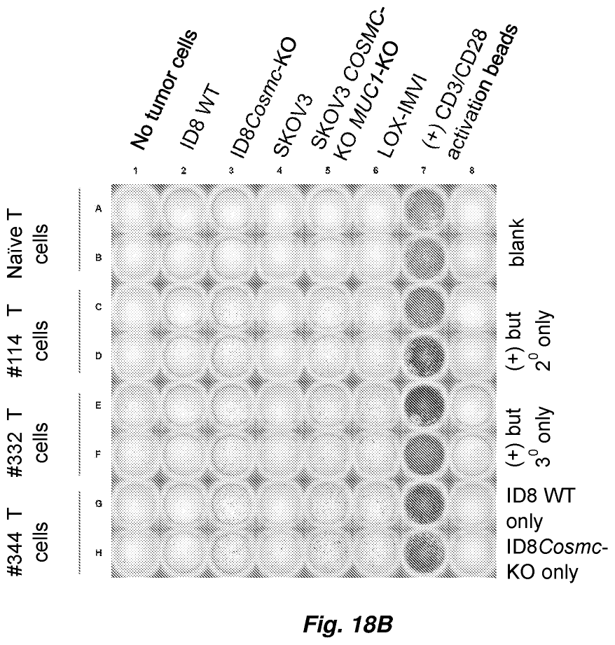

To assess persistence of CAR-T cells in long-term murine survivors, T cells purified from spleen of 237 CAR-T alone or PAC-1+237 CAR-T treated mice were co-cultured with Tn-expressing tumor cells for 24 hours (FIG. 18A-B). We performed IFN-γ elispot assay to measure T cell activation following target recognition. Consistent with our previous observations, a higher number of IFN-γ-secreting T cells were observed in response to ID8Cosmc-KO cell line compared to the non-Tn antigen expressing ID8 WT cell line. Similarly, the purified T cells were also able to recognize Tn antigen on human target cell lines such as SKOV3 COSMC-KO MUC1-KO and LOX-IMVI cell lines, but not against the wild-type non-Tn expressing SKOV3 parental cell line. This data suggest that even after 300+ days post-treatment administration of 237 CAR-T (+/−PAC-1) to the tumor-bearing host, there were still sufficient CAR-T cells present in the spleen that are ready to respond when presented with their target antigen.

Developing a CAR-T that Targets Human Tn-MUC1 in Solid Cancers.

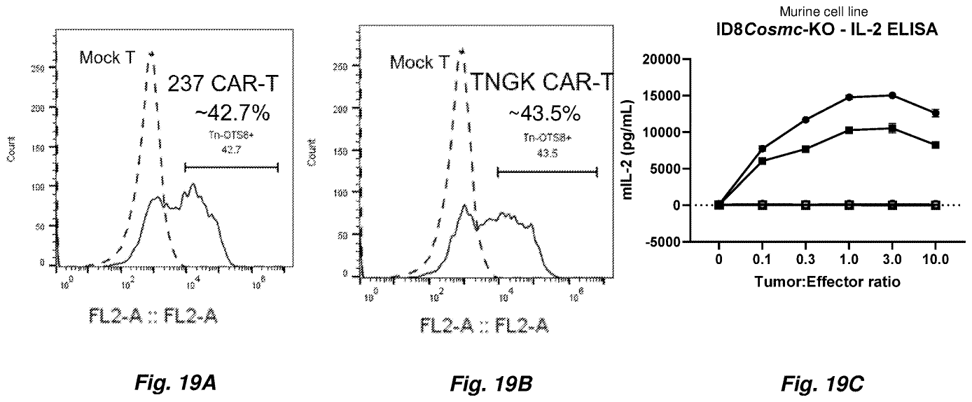

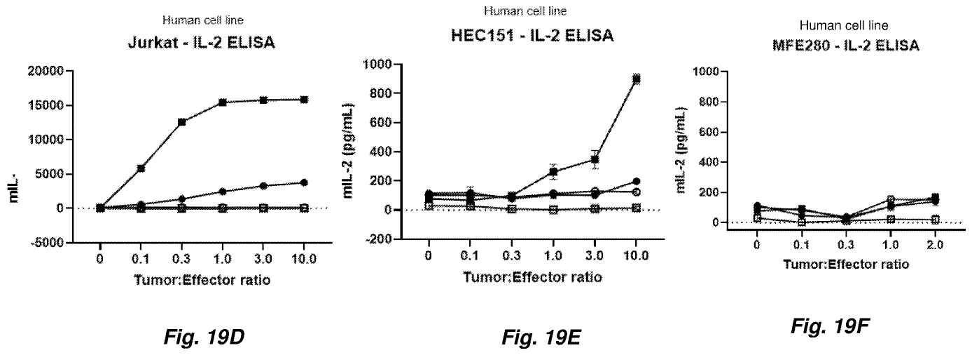

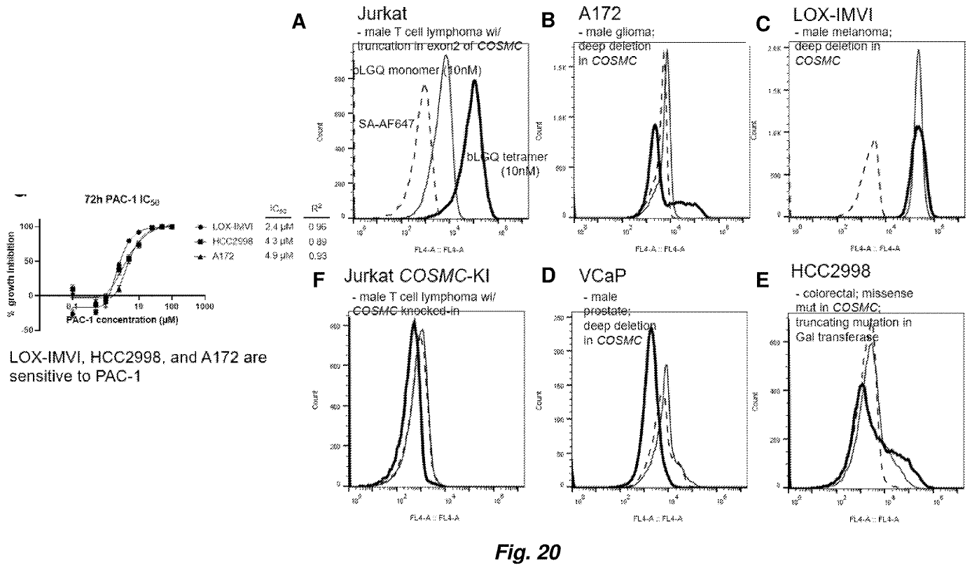

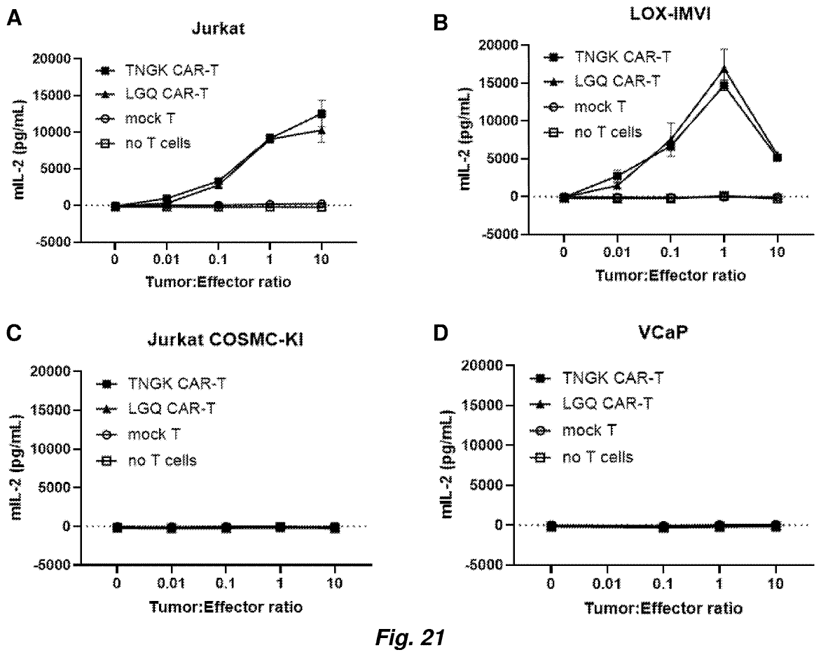

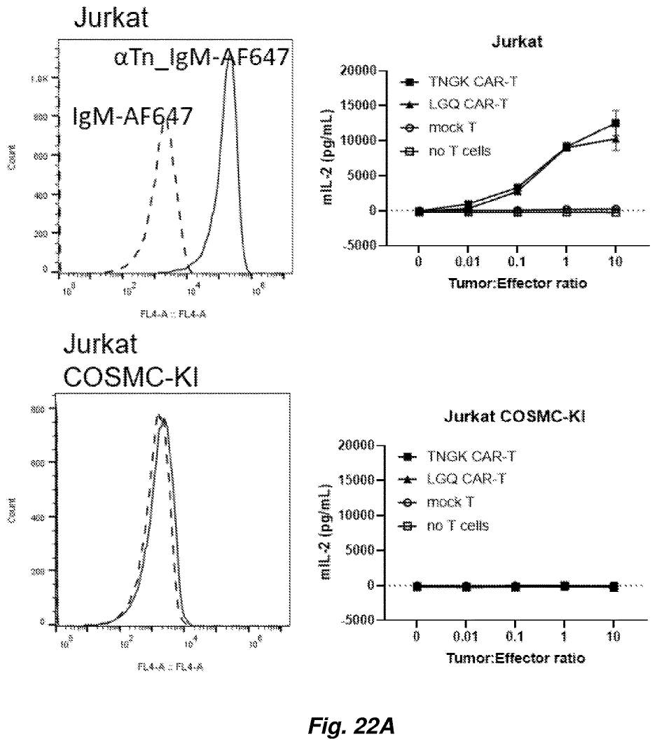

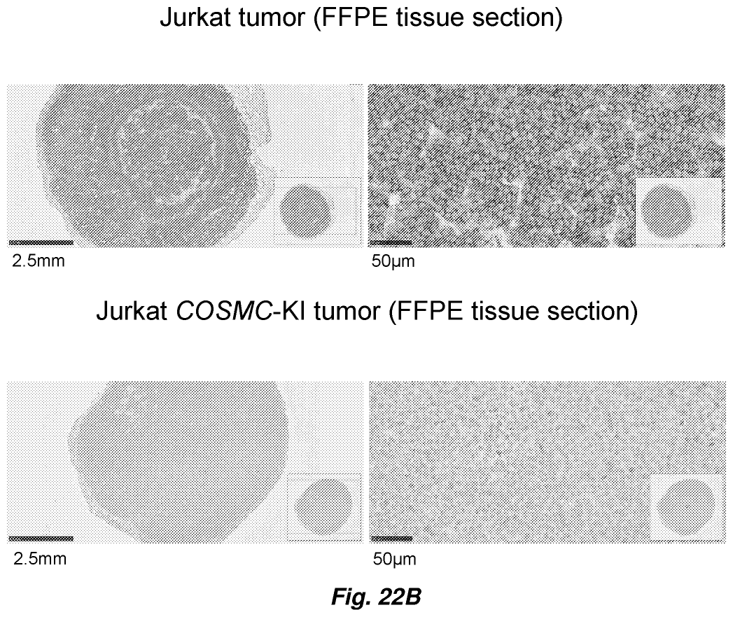

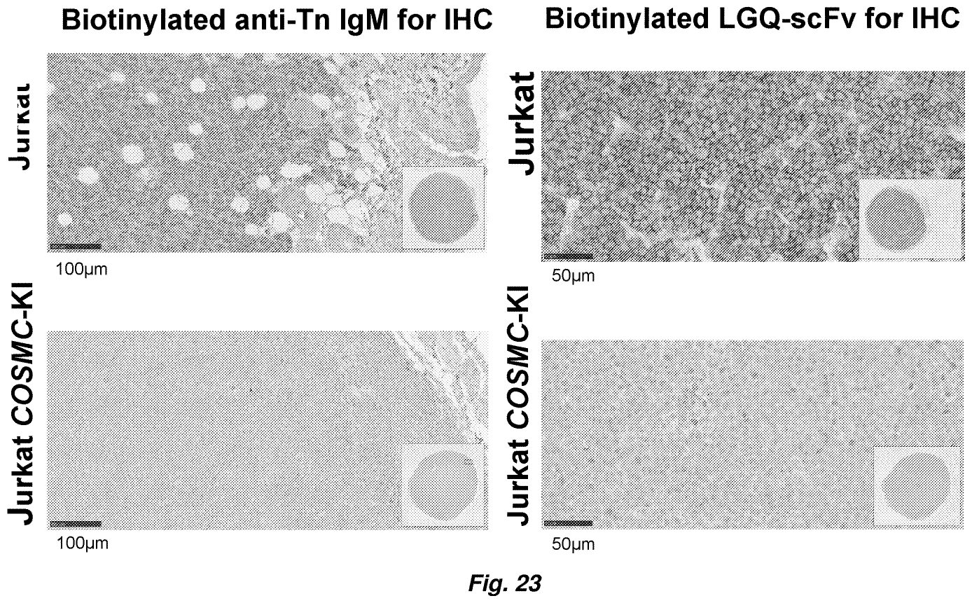

TNGK CAR was a derivative of 237 CAR engineered to have higher affinity towards Tn-MUC1 expressed on human tumors. In co-culture assays, 237 CAR-T has been shown to have stronger response against the murine target ID8Cosmc-KO compared to TNGK CAR-T, as measured by mIL-2 ELISA (FIG. 19A-F), but against the human target Jurkat, TNGK CAR-T was found to have higher response compared to 237 CAR-T. We screened several human cell lines with predicted deletion of the CIGALTIC1 gene using both co-culture assays (FIG. 19A-F) and flow cytometry staining for Tn antigen (FIG. 20A-F). We found two cell lines strongly positive for Tn-expression, the T-cell lymphoma line Jurkat and the melanoma line LOX-IMVI (FIGS. 20A and C). Both were recognized by TNGK CAR-T and LGQ CAR-T, an alternative engineered CAR derived from 237 CAR that can also recognize human targets, in co-culture experiments, using both IL-2 (FIG. 21A-F) and IFN-γ ELISA-based assays (data not shown) as read-outs. RNA sequencing confirmed a frameshift deletion of CIGALTIC1 exon 2 in Jurkat and the lack of CIGALTIC1 transcripts in LOX-IMVI, thus leading to a strong Tn antigen expression on their cell surface. We further queried the TCGA database for the frequency of CIGALTIC1 deep deletions in human cancer patients, and found 2-20% prevalence across different types of cancer, with urinary bladder cancer and soft tissue sarcoma on top. We are currently doing immunohistochemistry of commercially available formalin-fixed paraffin embedded (FFPE) biopsy samples from urinary bladder cancer patients in order to develop an approach that might support a phase I human clinical trial. Both purified biotinylated-LGQ scFv tetramers and biotinylated anti-Tn IgM may be employed as detection tools for Tn-antigen staining by flow cytometry and immunohistochemistry, using Jurkat and Jurkat COSMC-KI as positive and negative controls, respectively (FIGS. 22A-B, 23).

### Example 4. Exemplary Nucleic Acid Sequences Encoding

237 CAR fusion protein/scFv
(SEQ ID NO: 9)
gcggccgccaccatgggatggagttgtatcattctcttcctcgtcgctaccgccaccggagtgcattcagacattcagctcacccagtcacctctct
cactgccagtctctctcggcgaccaggctagtatctcatgcagaagctcccagtcactggtgcacagcaacggtaatacatacctgcattggtatct
ccagaagcccggacagagccctaagctgctcatctacaaagtctccaacagattctctggagtgccagacaggttcagcggatccggatctggg
actgatttcaccctgaagatttctagtgtcgaggccgaagacctcggcgtgtatttctgcagtcagtcaacacacgtgcctacttttggcggaggga
ccaagctggagatcaaaggtggcggagggagtggtggaggaggatcaggtggaggaggaagccaggtccagctgcagcagtctggtggag
gactcgtgcagcctggaggtagtatgaagattttctgtgccgcttccggattcaccttttctgacgcatggatggattgggtccgccagtctccagag
aaggggctggaatgggtggccgagatcaggaacaaagctaacaatcacgaaacatactatgccgagagcgtgaagggcaggttcaccattact
cgggacgatagcaaatcccgcatgagtctgcagatgaacagcctccgagcagaagacactggaatctactattgtagcggcggaaaggtccgc
aacgcctactggggtcagggcaccacagtcacagtgtcaagcctcgagaaggtgaacagcactaccacaaaaccagtcctgagaactcccag
ccctgtgcatccaaccgggacatcccagccacagcgaccagaggattgccgaccacgaggatccgtgaagggaaccgggctggacttcgcct
gtgattcctctcctaaactgttttgggctctcgtggtcgtggcaggagtgctgttttgctacgggctgctcgtcactgtggccctgtgcgtgatttgg
accaacagcaggagaaatcggctgctccaagtgactaccatgaacatgacccctcggcgcccaggcctgacaagaaagccctaccagccttatg
ccccagctcgggacttcgcagcatatcgaccagcacacgcacgagctaaattttctaggagtgctgaaacagctgcaaacctgcaggatcccaa
tcagctctacaacgagctgaatctcggacgaagggaggaatatgacgtgctggaaaagaaacgagctagggatcctgagatgggaggcaagc
agcagagacggcgcaacccacaggaaggcgtgtacaatgcactgcagaaggacaaaatggcagaagcctatagcgagattggaactaaggg
ggagcgaaggagaggtaaaggccatgatggtctgtaccagggcctgagcacagccacaaaggatacatacgatgccctccacatgcagacac
tcgcacccaggtgatgagaattc
WE CAR fusion protein/scFv
(SEQ ID NO: 10)
gcggccgccaccatgggatggagttgtatcattctcttcctcgtcgctaccgccaccggagtgcattcagacattcagctcacccagtcacctctct
cactgccagtctctctcggcgaccaggctagtatctcatgcagaagctcccagtcactggtgcacagcaacggtaatacatacctgcattggtatct
ccagaagcccggacagagccctaagctgctcatctacaaagtctccaacagattctctggagtgccagacaggttcagcggatccggatctggg
actgatttcaccctgaagatttctagtgtcgaggccgaagacctcggcgtgtatttctgcagtcagtcaacatgggagcctacttttggcggaggga
ccaagctggagatcaaaggtggcggagggagtggtggaggaggatcaggtggaggaggaagccaggtccagctgcagcagtctggtggag
gactcgtgcagcctggaggtagtatgaagattttctgtgccgcttccggattcaccttttctgacgcatggatggattgggtccgccagtctccagag
aaggggctggaatgggtggccgagatcaggaacaaagctaacaatcacgaaacatactatgccgagagcgtgaagggcaggttcaccattact
cgggacgatagcaaatcccgcatgagtctgcagatgaacagcctccgagcagaagacactggaatctactattgtagcggcggaaaggtccgc
aacgcctactggggtcagggcaccacagtcacagtgtcaagcctcgagaaggtgaacagcactaccacaaaaccagtcctgagaactcccag
ccctgtgcatccaaccgggacatcccagccacagcgaccagaggattgccgaccacgaggatccgtgaagggaaccgggctggacttcgcct
gtgattcctctcctaaactgttttgggctctcgtggtcgtggcaggagtgctgttttgctacgggctgctcgtcactgtggccctgtgcgtgatttgg
accaacagcaggagaaatcggctgctccaagtgactaccatgaacatgacccctcggcgcccaggcctgacaagaaagccctaccagccttatg
ccccagctcgggacttcgcagcatatcgaccagcacacgcacgagctaaattttctaggagtgctgaaacagctgcaaacctgcaggatcccaa
tcagctctacaacgagctgaatctcggacgaagggaggaatatgacgtgctggaaaagaaacgagctagggatcctgagatgggaggcaagc
agcagagacggcgcaacccacaggaaggcgtgtacaatgcactgcagaaggacaaaatggcagaagcctatagcgagattggaactaaggg
ggagcgaaggagaggtaaaggccatgatggtctgtaccagggcctgagcacagccacaaaggatacatacgatgccctccacatgcagacac
tcgcacccaggtgatgagaattc
TNGK CAR fusion protein/scFv
(SEQ ID NO: 11)
gcggccgccaccatgggatggagttgtatcattctcttcctcgtcgctaccgccaccggagtgcattcagacattcagctcacccagtcacctctct
cactgccagtctctctcggcgaccaggctagtatctcatgcagaagctcccagtcactggtgcacagcaacggtaatacatacctgcattggtatct
ccagaagcccggacagagccctaagctgctcatctacaaagtctccaacagattctctggagtgccagacaggttcagcggatccggatctggg
actgatttcaccctgaagatttctagtgtcgaggccgaagacctcggcgtgtatttctgcagtcagacgaatggcaagcctacttttggcggaggga
ccaagctggagatcaaaggtggcggagggagtggtggaggaggatcaggtggaggaggaagccaggtccagctgcagcagtctggtggag
gactcgtgcagcctggaggtagtatgaagattttctgtgccgcttccggattcaccttttctgacgcatggatggattgggtccgccagtctccagag
aaggggctggaatgggtggccgagatcaggaacaaagctaacaatcacgaaacatactatgccgagagcgtgaagggcaggttcaccattact
cgggacgatagcaaatcccgcatgagtctgcagatgaacagcctccgagcagaagacactggaatctactattgtagcggcggaaaggtccgc
aacgcctactggggtcagggcaccacagtcacagtgtcaagcctcgagaaggtgaacagcactaccacaaaaccagtcctgagaactcccag
ccctgtgcatccaaccgggacatcccagccacagcgaccagaggattgccgaccacgaggatccgtgaagggaaccgggctggacttcgcct
gtgattcctctcctaaactgttttgggctctcgtggtcgtggcaggagtgctgttttgctacgggctgctcgtcactgtggccctgtgcgtgatttgg
accaacagcaggagaaatcggctgctccaagtgactaccatgaacatgacccctcggcgcccaggcctgacaagaaagccctaccagccttatg
ccccagctcgggacttcgcagcatatcgaccagcacacgcacgagctaaattttctaggagtgctgaaacagctgcaaacctgcaggatcccaa
tcagctctacaacgagctgaatctcggacgaagggaggaatatgacgtgctggaaaagaaacgagctagggatcctgagatgggaggcaagc
agcagagacggcgcaacccacaggaaggcgtgtacaatgcactgcagaaggacaaaatggcagaagcctatagcgagattggaactaaggg
ggagcgaaggagaggtaaaggccatgatggtctgtaccagggcctgagcacagccacaaaggatacatacgatgccctccacatgcagacac
tcgcacccaggtgatgagaattc
LGQ CAR fusion protein/scFv
(SEQ ID NO: 12)
gcggccgccaccatgggatggagttgtatcattctcttcctcgtcgctaccgccaccggagtgcattcagacattcagctcacccagtcacctctct
cactgccagtctctctcggcgaccaggctagtatctcatgcagaagctcccagtcactggtgcacagcaacggtaatacatacctgcattggtatct
ccagaagcccggacagagccctaagctgctcatctacaaagtctccaacagattctctggagtgccagacaggttcagcggatccggatctggg
actgatttcaccctgaagatttctagtgtcgaggccgaagacctcggcgtgtatttctgcagtcagtcattaggccagcctacttttggcggaggga
ccaagctggagatcaaaggtggcggagggagtggtggaggaggatcaggtggaggaggaagccaggtccagctgcagcagtctggtggag
gactcgtgcagcctggaggtagtatgaagattttctgtgccgcttccggattcaccttttctgacgcatggatggattgggtccgccagtctccagag
aaggggctggaatgggtggccgagatcaggaacaaagctaacaatcacgaaacatactatgccgagagcgtgaagggcaggttcaccattact
cgggacgatagcaaatcccgcatgagtctgcagatgaacagcctccgagcagaagacactggaatctactattgtagcggcggaaaggtccgc
aacgcctactggggtcagggcaccacagtcacagtgtcaagcctcgagaaggtgaacagcactaccacaaaaccagtcctgagaactcccag
ccctgtgcatccaaccgggacatcccagccacagcgaccagaggattgccgaccacgaggatccgtgaagggaaccgggctggacttcgcct
gtgattcctctcctaaactgttttgggctctcgtggtcgtggcaggagtgctgttttgctacgggctgctcgtcactgtggccctgtgcgtgatttgg
accaacagcaggagaaatcggctgctccaagtgactaccatgaacatgacccctcggcgcccaggcctgacaagaaagccctaccagccttatg
ccccagctcgggacttcgcagcatatcgaccagcacacgcacgagctaaattttctaggagtgctgaaacagctgcaaacctgcaggatcccaa
tcagctctacaacgagctgaatctcggacgaagggaggaatatgacgtgctggaaaagaaacgagctagggatcctgagatgggaggcaagc
agcagagacggcgcaacccacaggaaggcgtgtacaatgcactgcagaaggacaaaatggcagaagcctatagcgagattggaactaaggg
ggagcgaaggagaggtaaaggccatgatggtctgtaccagggcctgagcacagccacaaaggatacatacgatgccctccacatgcagacac
tcgcacccaggtgatgagaattc

### Example 5. Pharmaceutical Dosage Forms

The following formulations illustrate representative pharmaceutical dosage forms that may be used for the therapeutic or prophylactic administration of a composition of a formula described herein, a composition specifically disclosed herein, or a pharmaceutically acceptable salt thereof (hereinafter referred to as ‘Composition X’):

| | mg/tablet |
|---|---|
| (i) Tablet 1 | |
| ‘Composition X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | |
| ‘Composition X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Composition X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| | mg/mL |
|---|---|
| (iv) Injection 1 (1 mg/mL) | |
| ‘Composition X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/mL) | |
| ‘Composition X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Composition X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the disclosure. Changes and modifications can be made in accordance with ordinary skill in the art without departing from the disclosure in its broader aspects as defined in the following claims.

All publications, patents, and patent documents are incorporated by reference herein, as though individually incorporated by reference, and in particular, Ranoa et al., J Immunother Cancer. 2023 May; 11(5):e006509. doi: 10.1136/jitc-2022-006509. PMID: 37258040; PMCID: PMC10255004; U.S. Pat. No. 11,129,830 to Hergenrother et al.; U.S. Pat. No. 11,384,133 to Smith et al.; U.S. Patent Publication No. 2019/0112380 to Chaudhary; U.S. Patent Publication No. 2017/0145108 to Schreiber et al., and U.S. Patent Publication No. 2021/0220404 to Abujoub et al. No limitations inconsistent with this disclosure are to be understood therefrom. The disclosure has been described with reference to various specific and preferred embodiments and techniques. However, it should be understood that many variations and modifications may be made while remaining within the spirit and scope of the disclosure.

What is claimed is:

1. A composition comprising:
the compound PAC-1: 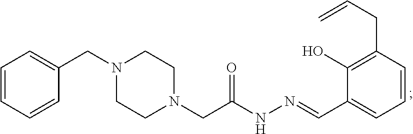 [SMILES: C=CCc1cccc(/C=N/NC(=O)CN2CCN(Cc3ccccc3)CC2)c1O]
and
a population of T-cells comprising a chimeric antigen receptor (CAR) fusion protein displayed on a cell surface membrane of the T-cells, wherein the CAR fusion protein selectively binds to a protein disposed on a surface of a tumor cell, wherein the protein comprises an N-acetylgalactosamine-O-Serine or N-acetylgalactosamine-O-Threonine antigen moiety (Tn-antigen).

2. The composition of claim 1 wherein the CAR fusion protein comprises one or more of a single chain variable fragment (scFv), a single domain antibody, a bispecific antibody, and a multispecific antibody.

3. The composition of claim 2 wherein the CAR fusion protein comprises a single-chain variable fragment (scFv) antibody that selectively binds to the Tn-antigen.

4. The composition of claim 1 wherein the CAR fusion protein further comprises a hinge region, a transmembrane domain, and a cytoplasmic domain.

5. The composition of claim 1 wherein the CAR fusion protein comprises a formula A-B-C-D, wherein A is the scFv, B is the hinge region, C is the transmembrane domain, and D is the cytoplasmic domain.

6. The composition of claim 1 wherein the CAR fusion protein comprises an amino acid sequence of SEQ ID NO: 7, SEQ ID NO: 5, SEQ ID NO: 6, or SEQ ID NO: 8.

7. The composition of claim 1 wherein the CAR fusion protein comprises an amino acid sequence of SEQ ID NO: 3, SEQ ID NO: 1, SEQ ID NO: 2, or SEQ ID NO: 4.

8. The composition of claim 4 wherein the hinge region is derived from a CD8 protein, the transmembrane domain is derived from a CD28 protein, and the cytoplasmic domain is derived from a CD3ζ protein.

9. The composition of claim 1 wherein the population of T-cells comprises about 5×10⁵ T-cells to about 5×10⁸ T-cells, or about 5×10⁶ T-cells.

10. The composition of claim 1 wherein the composition comprises a PAC-1 concentration of about 0.1 μM to about 50 μM.

11. The composition of claim 1 wherein the protein comprising the Tn-antigen moiety comprises one or more of MUC1, OTS8, CD19, BCMA, CD22, CD30, CD7, CD38, kappa light chain, CD20, GD2, HER2, IL-13Rα2, EGFR, mesothelin, claudin-18.2, and PSMA.

12. The composition of claim 1 further comprising and a pharmaceutically acceptable carrier or excipient, wherein:
a) the carrier comprises one or more of water, a buffer, a sugar, a cellulose, a cyclodextrin, polyethylene glycol, tocopherol, a liposome, a micelle, or a combination thereof; or
b) the excipient comprises one or more of a binder, a lubricant, a sorbent, a vehicle, a disintegrant, a preservative, or a combination thereof.

13. A method of treating cancer comprising administering to a subject in need thereof, concurrently or sequentially, a therapeutically effective amount of PAC-1 and a therapeutically effective amount of a population of chimeric antigen receptor (CAR) T-cells wherein the effect of the population of CAR T-cells is enhanced by the administration of the PAC-1, and
wherein the population of CAR T-cells comprise a CAR fusion protein that selectively binds to a protein disposed on a surface of a tumor cell, wherein the protein comprises an N-acetylgalactosamine-O-Serine or N-acetylgalactosamine-O-Threonine antigen moiety (Tn-antigen).

14. The method of claim 13 wherein the PAC-1 is administered in a dose comprising about 10 mg/kg to about 125 mg/kg; and/or the population of CAR T-cells are administered in a dose comprising about 5×10⁵ CAR T-cells to about 5×10⁸ Car T-cells.

15. The method of claim 13 wherein the cancer is embryonal tumor, esophagogastric cancer, central nervous system cancer, bladder cancer, breast cancer, glioblastoma, head and neck cancer, hepatobiliary cancer, lung cancer, melanoma, meningioma, multiple myeloma, ocular melanoma, ovarian cancer, ovarian epithelial cancer, osteosarcoma, pancreatic cancer, prostate cancer, renal cancer, salivary gland cancer, sarcoma, seminoma, or soft tissue sarcoma.

16. The method of claim 13 wherein:
the compound PAC-1 and the population of CAR T-cells are concurrently administered to the subject; or
the compound PAC-1 and the population of CAR T-cells are sequentially administered to the subject, wherein the compound PAC-1 is administered to the subject before the population of T-cells or the compound PAC-1 is administered to the subject after the population of CAR T-cells.

17. The method of claim 13 wherein the compound PAC-1 is administered to the subject once daily (q.d.), twice a day (b.i.d.), three times a day (t.i.d.), or four times a day (q.i.d.), wherein the total administered dose per day of PAC-1 is about 1 mg/kg to about 150 mg/kg; or
each administered dose of PAC-1 is about 70 mg, about 175 mg, about 250 mg, about 375 mg, about 450 mg, about 500 mg, about 625 mg, about 750 mg, or about 800 mg; or
each administered dose of PAC-1 is about 50 mg/m² to about 250 mg/m².

18. The method of claim 13 wherein the compound PAC-1 is administered over the course of three administration time periods, wherein each of the administration time periods is 5 consecutive days with at least two days between each of the administration time periods, wherein the PAC-1 is administered once daily for each of the 5 consecutive days; and
the population of T-cells are administered during one of a second or third administration time period.

19. The method of claim 13 wherein the protein comprising the Tn-antigen moiety comprises one or more of MUC1, OTS8, CD19, BCMA, CD22, CD30, CD7, CD38, kappa light chain, CD20, GD2, HER2, IL-13Rα2, EGFR, mesothelin, claudin-18.2, and PSMA.

20. A method of treating ovarian cancer comprising administering to a human in need thereof, concurrently or sequentially, a therapeutically effective amount of:
PAC-1; and
a population of T-cells comprising a chimeric antigen receptor (CAR) fusion protein displayed on a cell surface membrane a T-cell, wherein the CAR fusion protein comprises one or more of a TNGK single chain variable fragment (scFv) (SEQ IDNO: 3), a 237 single chain variable fragment (scFv) (SEQ ID NO: 1), a WE single chain variable fragment (scFv) (SEQ ID NO: 2), and an LGQ single chain variable fragment (scFv) (SEQ ID NO: 4); and
wherein each of the 237 scFv, the WE scFv, the LGQ scFv, and the TNGK scFv selectively bind to protein comprising an N-acetylgalactosamine-O-Serine or N-acetylgalactosamine-O-Threonine antigen disposed on a surface of an ovarian cancer cell, thereby treating the ovarian cancer.


SEQUENCE LISTING (12 sequences)

SEQ ID NO: 1 - protein, 505 aa, synthetic construct
AAATMGWSCIILFLVATATGVHSDIQLTQSPLSLPVSLGDQASISCRSSQSLVHSNGNTYLHWYLQKPGQSPKLLIYKVSNRFSGVPDRFSGSGSGTDFTLKISSVEAEDLGVYFCSQSTHVPTFGGGTKLEIKGGGGSGGGGSGGGGSQVQLQQSGGGLVQPGGSMKIFCAASGFTFSDAWMDWVRQSPEKGLEWVAEIRNKANNHETYYAESVKGRFTITRDDSKSRMSLQMNSLRAEDTGIYYCSGGKVRNAYWGQGTTVTVSSLEKVNSTTTKPVLRTPSPVHPTGTSQPQRPEDCRPRGSVKGTGLDFACDSSPKLFWALVVVAGVLFCYGLLVTVALCVIWTNSRRNRLLQVTTMNMTPRRPGLTRKPYQPYAPARDFAAYRPAHARAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 2 - protein, 505 aa, synthetic construct
AAATMGWSCIILFLVATATGVHSDIQLTQSPLSLPVSLGDQASISCRSSQSLVHSNGNTYLHWYLQKPGQSPKLLIYKVSNRFSGVPDRFSGSGSGTDFTLKISSVEAEDLGVYFCSQSTWEPTFGGGTKLEIKGGGGSGGGGSGGGGSQVQLQQSGGGLVQPGGSMKIFCAASGFTFSDAWMDWVRQSPEKGLEWVAEIRNKANNHETYYAESVKGRFTITRDDSKSRMSLQMNSLRAEDTGIYYCSGGKVRNAYWGQGTTVTVSSLEKVNSTTTKPVLRTPSPVHPTGTSQPQRPEDCRPRGSVKGTGLDFACDSSPKLFWALVVVAGVLFCYGLLVTVALCVIWTNSRRNRLLQVTTMNMTPRRPGLTRKPYQPYAPARDFAAYRPAHARAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 3 - protein, 505 aa, synthetic construct
AAATMGWSCIILFLVATATGVHSDIQLTQSPLSLPVSLGDQASISCRSSQSLVHSNGNTYLHWYLQKPGQSPKLLIYKVSNRFSGVPDRFSGSGSGTDFTLKISSVEAEDLGVYFCSQTNGKPTFGGGTKLEIKGGGGSGGGGSGGGGSQVQLQQSGGGLVQPGGSMKIFCAASGFTFSDAWMDWVRQSPEKGLEWVAEIRNKANNHETYYAESVKGRFTITRDDSKSRMSLQMNSLRAEDTGIYYCSGGKVRNAYWGQGTTVTVSSLEKVNSTTTKPVLRTPSPVHPTGTSQPQRPEDCRPRGSVKGTGLDFACDSSPKLFWALVVVAGVLFCYGLLVTVALCVIWTNSRRNRLLQVTTMNMTPRRPGLTRKPYQPYAPARDFAAYRPAHARAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 4 - protein, 505 aa, synthetic construct
AAATMGWSCIILFLVATATGVHSDIQLTQSPLSLPVSLGDQASISCRSSQSLVHSNGNTYLHWYLQKPGQSPKLLIYKVSNRFSGVPDRFSGSGSGTDFTLKISSVEAEDLGVYFCSQSLGQPTFGGGTKLEIKGGGGSGGGGSGGGGSQVQLQQSGGGLVQPGGSMKIFCAASGFTFSDAWMDWVRQSPEKGLEWVAEIRNKANNHETYYAESVKGRFTITRDDSKSRMSLQMNSLRAEDTGIYYCSGGKVRNAYWGQGTTVTVSSLEKVNSTTTKPVLRTPSPVHPTGTSQPQRPEDCRPRGSVKGTGLDFACDSSPKLFWALVVVAGVLFCYGLLVTVALCVIWTNSRRNRLLQVTTMNMTPRRPGLTRKPYQPYAPARDFAAYRPAHARAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 5 - protein, 8 aa, synthetic construct
SQSTHVPT

SEQ ID NO: 6 - protein, 8 aa, synthetic construct
SQSTWEPT

SEQ ID NO: 7 - protein, 8 aa, synthetic construct
SQTNGKPT

SEQ ID NO: 8 - protein, 8 aa, synthetic construct
SQSLGQPT

SEQ ID NO: 9 - DNA, 1527 bp, unidentified
  source 1..1527: mol_type = other DNA
gcggccgccaccatgggatggagttgtatcattctcttcctcgtcgctaccgccaccggagtgcattcagacattcagctcacccagtcacctctctcactgccagtctctctcggcgaccaggctagtatctcatgcagaagctcccagtcactggtgcacagcaacggtaatacatacctgcattggtatctccagaagcccggacagagccctaagctgctcatctacaaagtctccaacagattctctggagtgccagacaggttcagcggatccggatctgggactgatttcaccctgaagatttctagtgtcgaggccgaagacctcggcgtgtatttctgcagtcagtcaacacacgtgcctacttttggcggagggaccaagctggagatcaaaggtggcggagggagtggtggaggaggatcaggtggaggaggaagccaggtccagctgcagcagtctggtggaggactcgtgcagcctggaggtagtatgaagattttctgtgccgcttccggattcaccttttctgacgcatggatggattgggtccgccagtctccagagaaggggctggaatgggtggccgagatcaggaacaaagctaacaatcacgaaacatactatgccgagagcgtgaagggcaggttcaccattactcgggacgatagcaaatcccgcatgagtctgcagatgaacagcctccgagcagaagacactggaatctactattgtagcggcggaaaggtccgcaacgcctactggggtcagggcaccacagtcacagtgtcaagcctcgagaaggtgaacagcactaccacaaaaccagtcctgagaactcccagccctgtgcatccaaccgggacatcccagccacagcgaccagaggattgccgaccacgaggatccgtgaagggaaccgggctggacttcgcctgtgattcctctcctaaactgttttgggctctcgtggtcgtggcaggagtgctgttttgctacgggctgctcgtcactgtggccctgtgcgtgatttggaccaacagcaggagaaatcggctgctccaagtgactaccatgaacatgacccctcggcgcccaggcctgacaagaaagccctaccagccttatgccccagctcgggacttcgcagcatatcgaccagcacacgcacgagctaaattttctaggagtgctgaaacagctgcaaacctgcaggatcccaatcagctctacaacgagctgaatctcggacgaagggaggaatatgacgtgctggaaaagaaacgagctagggatcctgagatgggaggcaagcagcagagacggcgcaacccacaggaaggcgtgtacaatgcactgcagaaggacaaaatggcagaagcctatagcgagattggaactaagggggagcgaaggagaggtaaaggccatgatggtctgtaccagggcctgagcacagccacaaaggatacatacgatgccctccacatgcagacactcgcacccaggtgatgagaattc

SEQ ID NO: 10 - DNA, 1527 bp, synthetic construct
  source 1..1527: mol_type = other DNA
gcggccgccaccatgggatggagttgtatcattctcttcctcgtcgctaccgccaccggagtgcattcagacattcagctcacccagtcacctctctcactgccagtctctctcggcgaccaggctagtatctcatgcagaagctcccagtcactggtgcacagcaacggtaatacatacctgcattggtatctccagaagcccggacagagccctaagctgctcatctacaaagtctccaacagattctctggagtgccagacaggttcagcggatccggatctgggactgatttcaccctgaagatttctagtgtcgaggccgaagacctcggcgtgtatttctgcagtcagtcaacatgggagcctacttttggcggagggaccaagctggagatcaaaggtggcggagggagtggtggaggaggatcaggtggaggaggaagccaggtccagctgcagcagtctggtggaggactcgtgcagcctggaggtagtatgaagattttctgtgccgcttccggattcaccttttctgacgcatggatggattgggtccgccagtctccagagaaggggctggaatgggtggccgagatcaggaacaaagctaacaatcacgaaacatactatgccgagagcgtgaagggcaggttcaccattactcgggacgatagcaaatcccgcatgagtctgcagatgaacagcctccgagcagaagacactggaatctactattgtagcggcggaaaggtccgcaacgcctactggggtcagggcaccacagtcacagtgtcaagcctcgagaaggtgaacagcactaccacaaaaccagtcctgagaactcccagccctgtgcatccaaccgggacatcccagccacagcgaccagaggattgccgaccacgaggatccgtgaagggaaccgggctggacttcgcctgtgattcctctcctaaactgttttgggctctcgtggtcgtggcaggagtgctgttttgctacgggctgctcgtcactgtggccctgtgcgtgatttggaccaacagcaggagaaatcggctgctccaagtgactaccatgaacatgacccctcggcgcccaggcctgacaagaaagccctaccagccttatgccccagctcgggacttcgcagcatatcgaccagcacacgcacgagctaaattttctaggagtgctgaaacagctgcaaacctgcaggatcccaatcagctctacaacgagctgaatctcggacgaagggaggaatatgacgtgctggaaaagaaacgagctagggatcctgagatgggaggcaagcagcagagacggcgcaacccacaggaaggcgtgtacaatgcactgcagaaggacaaaatggcagaagcctatagcgagattggaactaagggggagcgaaggagaggtaaaggccatgatggtctgtaccagggcctgagcacagccacaaaggatacatacgatgccctccacatgcagacactcgcacccaggtgatgagaattc

SEQ ID NO: 11 - DNA, 1527 bp, synthetic construct
  source 1..1527: mol_type = other DNA
gcggccgccaccatgggatggagttgtatcattctcttcctcgtcgctaccgccaccggagtgcattcagacattcagctcacccagtcacctctctcactgccagtctctctcggcgaccaggctagtatctcatgcagaagctcccagtcactggtgcacagcaacggtaatacatacctgcattggtatctccagaagcccggacagagccctaagctgctcatctacaaagtctccaacagattctctggagtgccagacaggttcagcggatccggatctgggactgatttcaccctgaagatttctagtgtcgaggccgaagacctcggcgtgtatttctgcagtcagacgaatggcaagcctacttttggcggagggaccaagctggagatcaaaggtggcggagggagtggtggaggaggatcaggtggaggaggaagccaggtccagctgcagcagtctggtggaggactcgtgcagcctggaggtagtatgaagattttctgtgccgcttccggattcaccttttctgacgcatggatggattgggtccgccagtctccagagaaggggctggaatgggtggccgagatcaggaacaaagctaacaatcacgaaacatactatgccgagagcgtgaagggcaggttcaccattactcgggacgatagcaaatcccgcatgagtctgcagatgaacagcctccgagcagaagacactggaatctactattgtagcggcggaaaggtccgcaacgcctactggggtcagggcaccacagtcacagtgtcaagcctcgagaaggtgaacagcactaccacaaaaccagtcctgagaactcccagccctgtgcatccaaccgggacatcccagccacagcgaccagaggattgccgaccacgaggatccgtgaagggaaccgggctggacttcgcctgtgattcctctcctaaactgttttgggctctcgtggtcgtggcaggagtgctgttttgctacgggctgctcgtcactgtggccctgtgcgtgatttggaccaacagcaggagaaatcggctgctccaagtgactaccatgaacatgacccctcggcgcccaggcctgacaagaaagccctaccagccttatgccccagctcgggacttcgcagcatatcgaccagcacacgcacgagctaaattttctaggagtgctgaaacagctgcaaacctgcaggatcccaatcagctctacaacgagctgaatctcggacgaagggaggaatatgacgtgctggaaaagaaacgagctagggatcctgagatgggaggcaagcagcagagacggcgcaacccacaggaaggcgtgtacaatgcactgcagaaggacaaaatggcagaagcctatagcgagattggaactaagggggagcgaaggagaggtaaaggccatgatggtctgtaccagggcctgagcacagccacaaaggatacatacgatgccctccacatgcagacactcgcacccaggtgatgagaattc

SEQ ID NO: 12 - DNA, 1527 bp, synthetic construct
  source 1..1527: mol_type = other DNA
gcggccgccaccatgggatggagttgtatcattctcttcctcgtcgctaccgccaccggagtgcattcagacattcagctcacccagtcacctctctcactgccagtctctctcggcgaccaggctagtatctcatgcagaagctcccagtcactggtgcacagcaacggtaatacatacctgcattggtatctccagaagcccggacagagccctaagctgctcatctacaaagtctccaacagattctctggagtgccagacaggttcagcggatccggatctgggactgatttcaccctgaagatttctagtgtcgaggccgaagacctcggcgtgtatttctgcagtcagtcattaggccagcctacttttggcggagggaccaagctggagatcaaaggtggcggagggagtggtggaggaggatcaggtggaggaggaagccaggtccagctgcagcagtctggtggaggactcgtgcagcctggaggtagtatgaagattttctgtgccgcttccggattcaccttttctgacgcatggatggattgggtccgccagtctccagagaaggggctggaatgggtggccgagatcaggaacaaagctaacaatcacgaaacatactatgccgagagcgtgaagggcaggttcaccattactcgggacgatagcaaatcccgcatgagtctgcagatgaacagcctccgagcagaagacactggaatctactattgtagcggcggaaaggtccgcaacgcctactggggtcagggcaccacagtcacagtgtcaagcctcgagaaggtgaacagcactaccacaaaaccagtcctgagaactcccagccctgtgcatccaaccgggacatcccagccacagcgaccagaggattgccgaccacgaggatccgtgaagggaaccgggctggacttcgcctgtgattcctctcctaaactgttttgggctctcgtggtcgtggcaggagtgctgttttgctacgggctgctcgtcactgtggccctgtgcgtgatttggaccaacagcaggagaaatcggctgctccaagtgactaccatgaacatgacccctcggcgcccaggcctgacaagaaagccctaccagccttatgccccagctcgggacttcgcagcatatcgaccagcacacgcacgagctaaattttctaggagtgctgaaacagctgcaaacctgcaggatcccaatcagctctacaacgagctgaatctcggacgaagggaggaatatgacgtgctggaaaagaaacgagctagggatcctgagatgggaggcaagcagcagagacggcgcaacccacaggaaggcgtgtacaatgcactgcagaaggacaaaatggcagaagcctatagcgagattggaactaagggggagcgaaggagaggtaaaggccatgatggtctgtaccagggcctgagcacagccacaaaggatacatacgatgccctccacatgcagacactcgcacccaggtgatgagaattc